(19) United States
(12) Patent Application Publication
(10) Pub. No.: US 2026/0114115 A1
(43) Pub. Date: Apr. 23, 2026

(54) COMPOUND AND ORGANIC ELECTROLUMINESCENT DEVICE

(71) Applicant: Samsung Electronics Co., Ltd., Suwon-si, Gyeonggi-do (KR)

(72) Inventors: Eigo MIYAZAKI, Kanagawa (JP); Katsunori SHIBATA, Kanagawa (JP); Keisuke KORAI, Kanagawa (JP); Atsushi IMAMURA, Kanagawa (JP); Mitsunori ITO, Kanagawa (JP)

(21) Appl. No.: 18/921,173

(22) Filed: Oct. 21, 2024

(30) Foreign Application Priority Data
Oct. 20, 2023 (JP) 2023-180968
Jan. 24, 2024 (KR) 10-2024-0010785

(51) Int. Cl. H10K 50/12; C07D 209/90; C09K 11/06; H10K 85/30; H10K 85/40; H10K 85/60

(52) U.S. Cl. CPC H10K 50/12; C07D 209/90; C09K 11/06; H10K 85/346; H10K 85/40; H10K 85/622; H10K 85/626; H10K 85/633; H10K 85/654; H10K 85/6572; H10K 85/6574; C09K 2211/1044

(57) ABSTRACT

A compound that has a nitrogen-containing condensed ring structure represented by Formula (1):

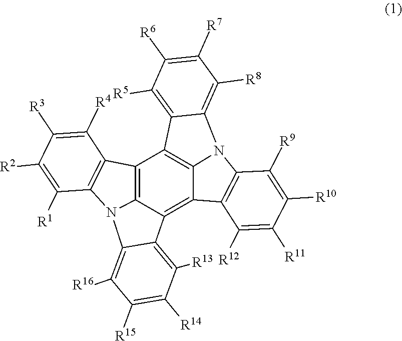

wherein Formula (1) is the same as described in the detailed description. In addition, provided is an organic electroluminescent device that has an emission layer including the compound having a nitrogen-containing condensed ring structure. Furthermore, provided is an organic electroluminescent device that has an emission layer including the compound having a nitrogen-containing condensed ring structure and a phosphorescent complex.

### CROSS-REFERENCE TO RELATED APPLICATIONS

This application claims priority to Korean Patent Application No. 10-2024-0010785, filed on Jan. 24, 2024, in the Korean Intellectual Property Office, and to Japanese Patent Application No. 2023-180968, filed on Oct. 20, 2023, in the Japanese Patent Office, and all benefits accruing therefrom under 35 U.S.C. § 119, the contents of which in their entirety are herein incorporated by reference.

### BACKGROUND

### 1. Field

The disclosure relates to a compound and an organic electroluminescent device including the compound.

### 2. Description of the Related Art

Recently, organic electroluminescent devices (hereinafter, also referred to as “organic EL devices”) have been used as various light-emitting devices including smartphones or televisions. Luminescent materials have been used in an emission layer of organic EL devices, and fluorescent materials, phosphorescent materials, and thermally activated delayed fluorescence materials have been reported as luminescent materials (for example, as disclosed in “Device Properties of Organic Semiconductors,” Edited by Chihaya Adachi, Kodansha, Mar. 22, 2012). Among organic EL devices, organic EL devices using fluorescent materials that utilize only fluorescence emission from a singlet have been put into practical use due to their luminescence principle, while, in typical organic EL devices, the luminescence efficiency may be 5% or less. For organic EL devices using phosphorescent materials, the luminescence efficiency of the devices may exceed 20%, and these organic EL devices have typically been put into practical use for green and red light emission. However, for blue light, fluorescence emission is generally used to provide satisfactory device lifespan, and improvement in performance is desirable.

Recently, BT.2100, which is a new international standard for television broadcasting, has been announced, and to comply with this standard, there is a demand for further improvement in color purity of the emission wavelength of light-emitting devices. Color purity may be improved and full width at half maximum (FWHM) of the emission wavelength may be narrowed, by introducing a microcavity structure into organic EL devices using conventional luminescent materials. However, in the case of light-emitting devices with a wide FWHM of the emission spectrum, the luminescence efficiency of the light-emitting devices may decrease because light that deviates from a target wavelength is not used. Therefore, there remains a demand for luminescent materials having a small FWHM of the emission spectrum and improved luminescence efficiency.

### SUMMARY

Disclosed is a compound that has a peak wavelength in an emission spectrum within a blue wavelength region and is capable of providing luminescence with high color purity and high efficiency. In addition, provided is an organic electroluminescent device having an emission layer including such a compound. Furthermore, the organic electroluminescent device has a peak wavelength in an emission spectrum within a blue wavelength region and is capable of providing luminescence with high color purity and high efficiency.

Additional aspects will be set forth in part in the description which follows and, in part, will be apparent from the description, or may be learned by practice of the presented embodiments of the disclosure.

The inventors have found that by using a compound having a specific nitrogen-containing condensed polycyclic structure, including such a compound in an emission layer of an organic electroluminescent device, and particularly by using a combination of such a compound and a phosphorescent material in the emission layer can provide the desired luminescent properties. Although the uses of such a compound are not limited to those described herein, such a compound may enable significant improvement to luminescence efficiency by being included in an emission layer, particularly by being included in combination with a phosphorescent material in the emission layer.

Provided is a compound represented by Formula (1).

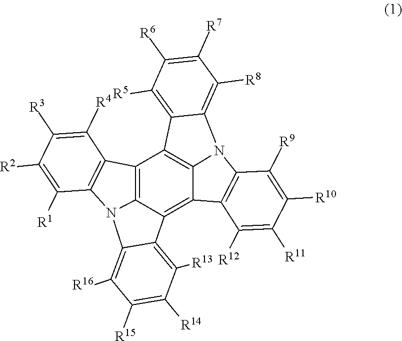

In Formula (1),
at least one of R¹ to R¹⁶ is a substituent W represented by Formula (2) (* indicates a binding site to a benzene ring), and

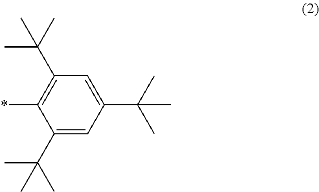 [SMILES: *c1c(C(C)(C)C)cc(C(C)(C)C)cc1C(C)(C)C]

R¹ to R¹⁶ other than the substituent W are each independently any one atom or group among (a1) to (a10):
(a1) a hydrogen or deuterium atom;
(a2) a halogen atom;
(a3) a cyano group;
(a4) a substituted or unsubstituted alkyl group having 1 to 20 carbon atoms;
(a5) a substituted or unsubstituted alkoxy group having 1 to 20 carbon atoms;
(a6) a substituted or unsubstituted arylamino group having 6 to 20 carbon atoms;
(a7) a substituted or unsubstituted triarylsilyl group, alkyldiarylsilyl group, dialkylarylsilyl group, or trialkylsilyl group (wherein an aryl group is an aryl group having 6 to 20 carbon atoms, and an alkyl group is an alkyl group having 1 to 20 carbon atoms);
(a8) a substituted or unsubstituted aromatic hydrocarbon group having 6 to 30 carbon atoms;
(a9) a substituted or unsubstituted heterocyclic group having 3 to 30 ring-forming atoms; and
(a10) a substituted or unsubstituted saturated hydrocarbon group or saturated heterocyclic group, each formed by bonding two adjacent groups among R¹ to R¹⁶ and having 5 to 9 ring-forming atoms.

Provided is the compound represented by Formula (1), wherein at least two of R², R³, R⁶, R⁷, R¹⁰, R¹⁴, R, and R¹⁵ may each be the substituent W.

Provided is an organic electroluminescent device including an emission layer including the compound represented by Formula (1).

Provided is the organic electroluminescent device including an emission layer may include a phosphorescent complex and the compound represented by Formula (1).

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view of an embodiment of an organic electroluminescent device;

FIG. 2 is a schematic cross-sectional view of an embodiment of an organic electroluminescent device;

FIG. 3 is a schematic cross-sectional view of an embodiment of an organic electroluminescent device according to another embodiment;

FIG. 4 is a graph illustrating photoluminescence intensity (arbitrary units, a.u.) versus wavelength (nanometers, nm) of an emission spectra of Compound 1 and Comparative Compound 1 in a toluene solution; and

FIG. 5 is a graph illustrating photoluminescence intensity (a.u.) versus wavelength (nm) of an emission spectrum of a thin film formed by co-depositing Compound 1 or Comparative Compound 1 with a host compound.

### DETAILED DESCRIPTION

Reference will now be made in detail to embodiments, examples of which are illustrated in the accompanying drawings, wherein like reference numerals refer to like elements throughout the specification. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the disclosure are described. In addition, the disclosure is not limited to the following embodiments, and various modifications may be made without departing from the scope as defined by the claims. In addition, the embodiments described herein may be arbitrarily combined to form other embodiments.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

As used herein, the expression “X-Y” is used to denote a range including values (X and Y) as the lower and upper bounds, respectively, and refers to “at least X and not more than Y.” As used herein, the expression “P and Q are each independently” refers to P and Q being identical to or different from each other. In addition, as used herein, the expression “A and/or B” refers to include each of A and B and a combination of A and B. In addition, unless specifically mentioned otherwise, the expressions “concentration” and “%” respectively refers to “mass concentration” or “wt %,” and unless specifically mentioned otherwise, the expression “ratio” refers to a weight ratio. In addition, unless specifically mentioned otherwise, measurements such as operations and properties are conducted under conditions of room temperature (20-25° C.)/relative humidity of 40-50% RH.

As used herein, the term “group derived from a ring” refers to a group having free valence by removing hydrogen atoms directly bonded to ring-forming atoms in a ring structure as much as a valence number. In this regard, ring-forming atoms represent atoms that directly form a ring structure. For example, in the case of a benzene ring, ring-forming atoms are carbon atoms, and hydrogen atoms are not included in the ring-forming atoms.

Recently, as a method of increasing the lifespan of organic EL devices and improving luminescence efficiency, use of organic EL devices in which a phosphor sensitizer and a luminescent material are combined for emission has been proposed (See Nature Communications, 2018, 9, 4990. DOI:10.1038/s41467-018-07432-2). In conventional organic EL devices, a host material and a luminescent material are used in an emission layer, and excitons generated in molecules of the host material within the emission layer transfer energy to a luminescent material, resulting in luminescence. Typically, when a fluorescent material is used as the luminescent material, the luminescence efficiency reaches a maximum of 5%. However, since triplet energy, which could not be used conventionally, may become available for luminescence when a phosphor sensitizer is added to the emission layer, the luminescence efficiency of organic EL devices may be improved by up to 10% or more. In addition, it has been reported that the device lifespan is longer than in the case of using a phosphor sensitizer as the luminescent material, and thus, organic EL devices using a phosphor sensitizer additionally to a luminescent material are attracting attention as candidates for the next-generation organic EL devices.

In order to reduce the FWHM of the emission spectrum of a luminescent material, it generally is necessary to suppress vibrational excitation within molecules and ensure a small change in conformation or bond length of the molecules between a ground state and an excited state. In order to suppress conformational change of the molecules, condensed compounds in which a bond distance between atoms is suppressed may be used. Synthesis and basic properties of a nitrogen-containing condensed polycyclic compound (S1) exhibiting a blue emission wavelength have been reported in Tetrahedron. 2013, 69, 3302-3307 and New J. Chem. 2010, 34, 1243-1246. These reports indicate that the nitrogen-containing condensed polycyclic compound (S1) is a promising luminescent material due to an emission spectrum having a small Stokes shift and a small FWHM in a solution state.

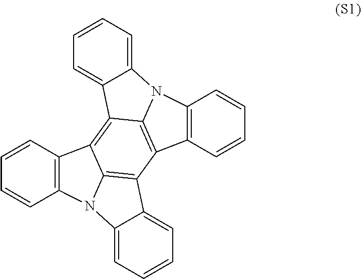 [SMILES: c1ccc2c(c1)c1c3c4ccccc4n4c5ccccc5c(c5c6ccccc6n2c15)c34]

As disclosed in WO2013/084805, when a derivative of the nitrogen-containing condensed polycyclic compound (S1) is used as an active layer of an organic transistor, the active layer exhibits p-type channel characteristics and high hole mobility. In addition, in JP 2020-107742, a derivative of the nitrogen-containing condensed polycyclic compound (S1), with an aryl group introduced as a substituent, functions as a luminescent material for organic EL devices, and the organic EL devices provide luminescence efficiency. As such, it may be seen that the nitrogen-containing condensed polycyclic compound (S1) is excellent as a basic skeleton for organic semiconductor materials. In JP 2020-107742, a manufactured organic EL device, which uses a derivative of the nitrogen-containing condensed polycyclic compound (S1) as a luminescent material in combination with a phosphorescent complex to provide a luminescence efficiency of 5% or more, also emits blue light with a FWHM of 20 nm or less and has a narrow FWHM.

However, in order to put a derivative using a nitrogen-containing condensed polycyclic compound (S1) as a basic skeleton into practical use as an organic EL device, further improvement in luminescence efficiency is needed.

According to the disclosure, provided is a compound capable of providing high efficiency for organic electroluminescent (EL) devices without impairing optical characteristics. A method for facilitating organic EL devices with high efficiency and without impaired optical characteristics of the luminescent materials in consideration of existing technology is also disclosed. A compound, in which a 2,4,6-tri-tert-butylphenyl group (a “substituent W”, by Formula (2)) that may be expected to have a high aggregation inhibition effect and is a bulky substituent is introduced into a nitrogen-containing condensed polycyclic compound (S1). The compound is represented by Formula (1). The compound represented by Formula (1) enables provision of: a blue luminescent material having a peak emission wavelength of about 440 nm to about 480 nm and achieving luminescence with a narrow spectrum having an emission spectrum width (full width at half maximum (FWHM)) of 20 nm or less; a composition utilizing the blue luminescent material; an organic EL device; and an organic EL display including the organic EL device. In addition, in the disclosure, hereinafter, the substituent represented by Formula (2) may be also referred to interchangeably as the “substituent W” or the “2,4,6-tri-tert-butylphenyl group.”

### Compound Represented by Formula (1)

The disclosure relates to a compound represented by Formula (1):

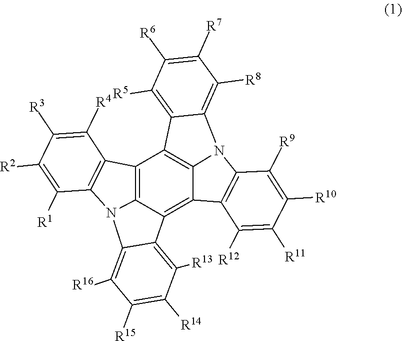

wherein, in Formula (1),
at least one of R¹ to R¹⁶ may be a substituent W represented by Formula (2) (* indicates a binding site to a benzene ring), and

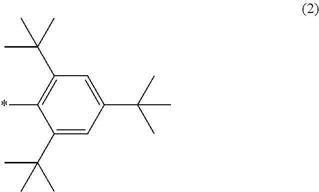 [SMILES: *c1c(C(C)(C)C)cc(C(C)(C)C)cc1C(C)(C)C]

R¹ to R¹⁶ other than the substituent W may each independently be any one atom or group among (a1) to (a10):
(a1) a hydrogen or deuterium atom;
(a2) a halogen atom;
(a3) a cyano group;
(a4) a substituted or unsubstituted alkyl group having 1 to 20 carbon atoms;
(a5) a substituted or unsubstituted alkoxy group having 1 to 20 carbon atoms;
(a6) a substituted or unsubstituted arylamino group having 6 to 20 carbon atoms;
(a7) a substituted or unsubstituted triarylsilyl group, alkyldiarylsilyl group, dialkylarylsilyl group, or trialkylsilyl group (wherein an aryl group is an aryl group having 6 to 20 carbon atoms, and an alkyl group is an alkyl group having 1 to 20 carbon atoms);
(a8) a substituted or unsubstituted aromatic hydrocarbon group having 6 to 30 carbon atoms;
(a9) a substituted or unsubstituted heterocyclic group having 3 to 30 ring-forming atoms; and
(a10) a substituted or unsubstituted saturated hydrocarbon group or saturated heterocyclic group, each formed by bonding two adjacent groups among R¹ to R¹⁶ and having 5 to 9 ring-forming atoms.

Hereinafter, the compound represented by Formula (1) according to the disclosure is also referred to as the term “compound of Formula (1).”

Not wishing to be bound by theory, it is believed that the emission wavelength of a luminescent material varies depending on not only the skeletal structure but also the type of substituent. As a result of the introduction of the specific substituent (substituent W), the compound of Formula (1) is long-wave-shifted to have a peak wavelength in the emission spectrum within a blue wavelength region. As a result, the compound of Formula (1) provides satisfactory characteristics as a luminescent material, in particular, as a blue luminescent material, for organic EL devices. In particular, the introduction of the substituent inhibits intermolecular aggregation and improves the solubility of the molecules themselves, leading to an improved degree of precision of purification. Consequently, the color purity of luminescence is also improved.

When the nitrogen-containing condensed polycyclic compound (S1) with high planarity is used as a luminescent material for organic EL devices, the distance between the nitrogen-containing condensed polycyclic compound (S1) and an adjacent host molecule within an emission layer decreases, thereby facilitating Dexter-type energy transfer. In addition, because the nitrogen-containing condensed polycyclic compound (S1) is a planar molecule, aggregation between luminescent materials may easily occur, and it is considered that the aggregation leads to degradation in color purity of the emission spectrum. Therefore, as a means to solve these issues, a method of introducing a sterically bulky substituent into the nitrogen-containing condensed polycyclic compound (S1) has been derived.

A mesityl group, a p-tert-butylphenyl group, and a 3,5-di-tert-butylphenyl group are known as bulky substituents, and compounds having these substituents introduced into their molecular skeletons or aryl groups of diarylamino groups are reported as luminescent materials (for example, JP 2012-176928, WO 2017/188111, and the like).

These bulky substituents are generally introduced by the Suzuki-Miyaura coupling reaction or Buchwald-Hartwig reaction to form carbon-carbon bonds or carbon-nitrogen bonds. When these substituents are introduced into compounds, a substituent at the ortho-position (o-position) of an aryl group, such as a mesityl group, is typically not bulky and thus does not hinder the reaction, to facilitate ease of introduction of an o-position substituent.

Introduction of a 2,4,6-tri-tert-butylphenyl group into the nitrogen-containing condensed polycyclic compound (S1) for the purpose of forming a compound having a more bulky substituent was considered. However, attempts to synthesize such a compound by introducing the 2,4,6-tri-tert-butylphenyl group (substituent W) into the nitrogen-containing condensed polycyclic compound (S1) via the Suzuki-Miyaura coupling reaction were unsuccessful and the desired compound could not be obtained. It is believed that the Suzuki-Miyaura coupling reaction did not proceed due to the bulky tert-butyl group present at the o-position of the phenyl group in the 2,4,6-tri-tert-butylphenyl group.

The inventors have discovered that by using a Negishi coupling reaction, it is possible to introduce the 2,4,6-tri-tert-butylphenyl group (substituent W) into the nitrogen-containing condensed polycyclic compound (S1) and prepare the compound of Formula (1). Furthermore, the inventors have demonstrated that the compound of Formula (1) exhibits excellent luminescence characteristics.

For example, there are four sites, at which substituents may be introduced, in each benzene ring (four benzene rings other than the central benzene ring) located on the outer portion of the compound of Formula (1) (e.g., R¹ to R¹⁶). The substitution positions of these benzene rings are numbered from positions 1 to 4 in order from the side closest to a condensed carbon atom closest to a nitrogen atom (for example, in the benzene ring having R¹ to R⁴, R¹ is the position 1, R² is the position 2, R³ is the position 3, and R⁴ is the position 4; in the benzene ring having R⁵ to R⁸, R⁵ is the position 1, R⁶ is the position 2, R⁷ is the position 3, and R⁸ is the position 4). In the disclosure, the compound of Formula (1) may exhibit excellent optical characteristics even when the substituent W is introduced at any position of a benzene ring. The substituent W may be at either the position 3 or the position 4, and for example, the substituent W may be at either the position 2 or the position 3 in each of two benzene rings facing each other.

As described above, according to the compound of Formula (1), it is assumed that Dexter-type energy transfer is suppressed because the substituent W is bulky, making it difficult for molecules of the compound of Formula (1) to be close to each other. Accordingly, aggregation between molecules of the compound of Formula (1) is inhibited. In general luminescent materials, intermolecular aggregation can lead to luminescence originating from an aggregated state, resulting in a tendency for the emission spectrum to broaden and color purity to degrade. However, because intermolecular aggregation is unlikely to occur in the compound of Formula (1), degradation in color purity is also unlikely to occur, and luminescence with high color purity may be realized. In addition, as a result, luminescence efficiency may be improved. When the concentration of the compound of Formula (1) is increased, it may become easier for aggregation to occur. However, even when the concentration of the compound of Formula (1) is increased, aggregation may remain inhibited such that luminescence with high color purity and high efficiency may be realized. In addition, when both the compound of Formula (1) and a phosphorescent complex are used, a significant increase in efficiency of organic EL devices may be realized. As described above, the use of a luminescent material of the compound of Formula (1) in organic EL devices may provide superior optical characteristics and high luminescence efficiency.

The above presented mechanism is based on an assumption and whether the assumed mechanism is right or wrong does not affect the technical scope of the disclosure. In addition, regarding other assumptions made herein, whether such assumptions are right or wrong does not affect the technical scope of the disclosure.

As such, an aspect of the disclosure relates to the compound represented by Formula (1). In addition, another aspect of the disclosure relates to an organic EL device having an emission layer including the compound represented by Formula (1). In addition, another aspect of the disclosure relates to an organic EL device having an emission layer including the compound represented by Formula (1) and a phosphorescent complex.

Hereinafter, the compound represented by Formula (1) according to an embodiment and the compound represented by Formula (1) included in the emission layer of the organic EL device according to an embodiment are described.

In Formula (1), at least one of R¹ to R¹⁶ is the substituent W represented by Formula (2) (2,4,6-tri-tert-butylphenyl group):

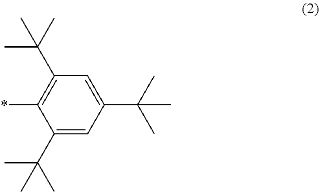 [SMILES: *c1c(C(C)(C)C)cc(C(C)(C)C)cc1C(C)(C)C]

In Formula (2), * indicates a binding site to a benzene ring of Formula (1). In the present embodiment, two of R¹ to R¹⁶ may each be the substituent W (2,4,6-tri-tert-butylphenyl group). Any of R¹ to R¹⁶ may be the substituent W, for example, at least two of R², R³, R⁶, R⁷, R¹⁰, R, R¹⁴, and R¹⁵ in Formula (1) may each be the substituent W. In addition, when two of R¹ to R¹⁶ are each the substituent W, the substituted groups may be a combination of, for example, R² and R¹⁰; R³ and R¹¹; R⁶ and R¹⁴; R⁷ and R¹⁵, or a combination thereof.

In Formula (1), R¹ to R¹⁶ other than the substituent W may each independently be any one atom or group among (a1) to (a10):
(a1) a hydrogen or deuterium atom;
(a2) a halogen atom;
(a3) a cyano group;
(a4) a substituted or unsubstituted alkyl group having 1 to 20 carbon atoms;
(a5) a substituted or unsubstituted alkoxy group having 1 to 20 carbon atoms;
(a6) a substituted or unsubstituted arylamino group having 6 to 20 carbon atoms;
(a7) a substituted or unsubstituted triarylsilyl group, alkyldiarylsilyl group, dialkylarylsilyl group, or trialkylsilyl group (wherein an aryl group is an aryl group having 6 to 20 carbon atoms, and an alkyl group is an alkyl group having 1 to 20 carbon atoms);
(a8) a substituted or unsubstituted aromatic hydrocarbon group having 6 to 30 carbon atoms;
(a9) a substituted or unsubstituted heterocyclic group having 3 to 30 ring-forming atoms; and
(a10) a substituted or unsubstituted saturated hydrocarbon group or saturated heterocyclic group, each formed by bonding two adjacent groups among R¹ to R¹⁶ and having 5 to 9 ring-forming atoms.

In Formula (1), from among atoms or groups of (a1) to (a9), preferably, the atoms or groups of (a1), (a4), (a7), (a8), and (a10) may be selected, and more preferably, the atoms or groups of (a1), (a4), and (a10) may be selected.

In Formula (1), when the groups of (a3) to (a10) are substituted groups, substituents that are substituted into such groups are not particularly limited. However, in Formula (1), substituents that are substituted into the groups of (a3) to (a10) may each independently be at least one substituent selected from a halogen atom, a cyano group, a substituted or unsubstituted alkyl group having 1 to 20 carbon atoms, a substituted or unsubstituted haloalkyl group having 1 to 20 carbon atoms, a substituted or unsubstituted alkoxy group having 1 to 20 carbon atoms, a substituted or unsubstituted alkylamino group having 1 to 20 carbon atoms, a substituted or unsubstituted arylamino group having 6 to 20 carbon atoms, a substituted or unsubstituted aromatic hydrocarbon group having 6 to 30 carbon atoms, and a substituted or unsubstituted heterocyclic group having 3 to 30 ring-forming atoms.

In the case of the hydrogen atom of (a1), a benzene ring is in an unsubstituted state. In addition, among stable isotopes of hydrogen, (a) may include a deuterium atom which contains one proton and one neutron in its nucleus. A deuterium atom is written as ²H or D (the initial letter of deuterium).

The halogen atom of (a2) is not particularly limited, and examples thereof include a fluorine atom (F), a chlorine atom (Cl), a bromine atom (Br), or an iodine atom (I). In particular, the halogen group may be a fluorine atom in view of luminescence efficiency.

The cyano group of (a3) is written as CN.

The alkyl group having 1 to 20 carbon atoms of (a4) is not particularly limited, and may be linear, branched, or cyclic. In particular, the alkyl group may be branched in view of luminescence color purity. The number of carbon atoms in the alkyl group may be 2 or more, for example, 3 or more, or for example, 4 or more, in view of solubility and luminescence color purity. In addition, the number of carbon atoms in the alkyl group may be 10 or less, for example, 8 or less, or for example, 6 or less, in view of luminescence efficiency. The number of carbon atoms in the alkyl group may be 4 in the above point of view. Examples of the alkyl group may include, but are not particularly limited to, a methyl group, an ethyl group, an n-propyl group, an iso-propyl group, an n-butyl group, a sec-butyl group (s-butyl group), a tert-butyl group (t-butyl group), an i-butyl group, a 2-ethyl butyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldodecyl group, a 2-octyldecyl group, an n-tridecyl group, an n-tetradecyl group, an n-pentadecyl group, an n-hexadecyl group, a 2-ethylhexadecyl group, a 2-butylhexadecyl group, an n-heptadecyl group, an n-octadecyl group, an n-nonadecyl group, an n-eicosyl group, or the like. In particular, the alkyl group may be a branched alkyl group, an isopropyl group, or a tert-butyl group, for example, a tert-butyl group.

In addition, the term “substituted alkyl group having 1 to 20 carbon atoms” refers to a group formed by substituting an unsubstituted alkyl group having 1 to 20 carbon atoms with a substituent. Therefore, the number of carbon atoms in the substituted alkyl group may be greater than 20.

The alkoxy group having 1 to 20 carbon atoms of (a5) is not particularly limited, and the alkoxy group may be linear, branched, or cyclic. In particular, the alkoxy group may be linear in view of luminescence efficiency. The number of carbon atoms in the alkoxy group may be 1 to 10 in view of luminescence efficiency. In the same point of view, the number of carbon atoms in the alkoxy group may be 1 to 8, for example, 1 to 6, or for example, 1. Examples of an alkyl group constituting the alkoxy group may include, but are not particularly limited to, those mentioned in the above description of the alkyl group. Examples of the alkoxy group may include, but are not particularly limited to, a methoxy group, an ethoxy group, an n-propoxy group, an iso-propoxy group, a butoxy group, a pentyloxy group, a hexyloxy group, a pctyloxy group, a nonyloxy group, a decyloxy group, or the like. In particular, the alkoxy group may be a methoxy group.

In addition, the term “substituted alkoxy group having 1 to 20 carbon atoms” refers to a group formed by substituting an unsubstituted alkoxy group having 1 to 20 carbon atoms with a substituent. Therefore, the number of carbon atoms in the substituted alkoxy group may be greater than 20.

A nitrogen atom of the arylamino group having 6 to 20 carbon atoms of (a6) may be bonded, via a single bond, to a ring-forming carbon atom of a benzene ring (among four benzene rings other than the central benzene ring) located on the outer side of the compound of Formula (1). In addition, herein, even in a case where a group includes a nitrogen atom, when the nitrogen atom is a ring-forming atom of a hetero ring, the group is handled as a heterocyclic group to be described below, instead of an arylamino group. Examples of an aryl group constituting the arylamino group may include, but are not particularly limited to, for example, a group having 6 to 20 carbon atoms among the aromatic hydrocarbon groups of (a8). The arylamino group may be, but is not particularly limited to, a monoarylamino group or a diarylamino group. Examples of the arylamino group may include, but are not particularly limited to, a N-phenylamino group, a N-biphenylamino group, a N-terphenylamino group, a N,N-diphenylamino group, a N-biphenyl-N-phenylamino group, or the like.

In addition, the term “substituted arylamino group having 6 to 20 carbon atoms” refers to a group formed by substituting an unsubstituted arylamino group having 6 to 20 carbon atoms with a substituent. Therefore, the number of carbon atoms in the substituted arylamino group may be greater than 20.

A silicon (Si) group of the triarylsilyl group, alkyldiarylsilyl group, dialkylarylsilyl group, or trialkylsilyl group of (a7) may be bonded, via a single bond, to a ring-forming carbon atom of a benzene ring (among four benzene rings other than the central benzene ring) located on the outer side of the compound of Formula (1). An aryl group constituting the triarylsilyl group, the alkyldiarylsilyl group, and the dialkylarylsilyl group may be an aryl group having 6 to 20 carbon atoms, and examples thereof may include a group having 6 to 20 carbon atoms among aromatic hydrocarbon groups to be described below. An alkyl group constituting the alkyldiarylsilyl group, the dialkylarylsilyl group, and the trialkylsilyl group may be an alkyl group having 1 to 20 carbon atoms, and examples thereof may be the same as the examples of the alkyl group having 1 to 20 carbon atoms of (a4). Examples of the triarylsilyl group may include, but are not particularly limited to, a triphenylsilyl group, a tri(tert-butylphenyl)silyl group, a di-tert-butylphenyl(phenyl)silyl group, or the like. Examples of the alkyldiarylsilyl group may include, but are not particularly limited to, a diphenylmethylsilyl group, a diphenyl(tert-butyl)silyl group, a di-tert-butylphenyl(methyl)silyl group, a di-tert-butylphenyl(tert-butyl)silyl group, or the like. Examples of the dialkylarylsilyl group may include, but are not particularly limited to, a dimethylphenylsilyl group, or the like. Examples of the trialkylsilyl group may include, but are not particularly limited to, a trimethylsilyl group, a tri-tert-butylsilyl group, a di-tert-butyl(methyl)silyl group, or the like.

In addition, the term “triarylsilyl group, alkyldiarylsilyl group, dialkylarylsilyl group, or trialkylsilyl group, each substituted” refers to a group formed by substituting an unsubstituted aryl group having 6 to 20 carbon atoms and an unsubstituted alkyl group having 1 to 20 carbon atoms with substituents. Therefore, the number of carbon atoms of an aryl group of the substituted triarylsilyl group, alkyldiarylsilyl group, and dialkylarylsilyl group may be greater than 20, and the number of carbon atoms of an alkyl group of the substituted alkyldiarylsilyl group, dialkylarylsilyl group, and trialkylsilyl group may be greater than 20.

The aromatic hydrocarbon group having 6 to 30 carbon atoms of (a8) refers to a group derived from at least one aromatic hydrocarbon ring. The term “aromatic hydrocarbon ring” as used herein refers to a hydrocarbon ring that is partially or entirely aromatic.

When the aromatic hydrocarbon group includes at least two aromatic hydrocarbon rings, these rings may be bonded to each other via a single bond or condensed to each other. In addition, when the aromatic hydrocarbon group includes at least two aromatic hydrocarbon rings, one atom may serve as a ring-forming atom of any of these rings.

In view of luminescence color purity, the number of carbon atoms in the aromatic hydrocarbon group may be 6 to 20, for example, 6 to 12, or for example, 6.

Examples of the aromatic hydrocarbon group may include, but are not particularly limited to, a phenyl group, a mesityl group, a tert-butylphenyl group, a bis(tert-butyl)phenyl group, a biphenyl group, a terphenyl group, a naphthyl group, a fluorenyl group, an anthracenyl group, a terphenyl group, a quaterterphenyl group, a quinquephenyl group, a sexiphenyl group, a triphenylenyl group, a pyrenyl group, a benzofluorenyl group, a chrysenyl group, or a combination thereof.

In addition, the term “substituted aromatic hydrocarbon group” refers to a group formed by substituting an unsubstituted aromatic hydrocarbon group with a substituent. Therefore, when the number of carbon atoms in the aromatic hydrocarbon group is equal to or less than a certain upper limit of the number of carbon atoms, for example, 30 or less, the number of carbon atoms in the substituted aromatic hydrocarbon group may be greater than the upper limit.

The heterocyclic group having 3 to 30 ring-forming atoms of (a9) refers to a group derived from at least one heteroatom-containing ring. The heterocyclic group may be, but is not particularly limited to, an aromatic heterocyclic group or a non-aromatic heterocyclic group. In particular, the heterocyclic group may be an aromatic heterocyclic group in view of luminescence color purity.

The term “aromatic heterocyclic group” refers to a group derived from at least one aromatic heteroatom-containing ring. The term “aromatic hetero ring” as used herein refers to a hetero ring that is partially or entirely aromatic. When the aromatic hetero ring is partially aromatic, aromaticity may be derived from a heterocyclic part of the ring or from a hydrocarbon ring part of the ring. The aromatic hetero ring is not particularly limited, and may be, for example, a ring having at least one heteroatom (e.g., a nitrogen atom (N), an oxygen atom (O), a phosphorus atom (P), a sulfur atom (S), or a silicon atom (Si)) as a ring-forming atom, wherein the remaining ring-forming atoms are carbon atoms (C). An atom constituting a ring structure may be bonded to an exocyclic atom via a double bond. For example, a carbon atom constituting the ring structure may constitute a ketone group (C═O group), a thioketone group (C═S group), or a C═NH group, or a sulfur atom constituting the ring structure may constitute a sulfinyl group (S═O group) or a sulfonyl group (S(═O)═O group). In this case, as used herein, the exocyclic atom forming the double bond with the atom constituting the ring structure may be part of the aromatic hetero ring. In addition, when the exocyclic atom forming the double bond is bonded to a hydrogen atom via a single bond, the hydrogen atom may also be part of the aromatic hetero ring. Examples of the aromatic hetero ring may include, but are not particularly limited to, a pyridine ring, a pyrazine ring, a pyridazine ring, a pyrimidine ring, a triazine ring, a quinoline ring, an isoquinoline ring, a quinoxaline ring, a quinazoline ring, a naphthyridine ring, an acridine ring, a phenazine ring, a benzoquinoline ring, a benzoisoquinoline ring, a phenanthridine ring, a phenanthroline ring, a benzoquinone ring, a coumarin ring, an anthraquinone ring, a fluorenone ring, a furan ring, a thiophene ring, a benzofuran ring, a benzothiophene ring, a dibenzofuran ring, a dibenzothiophene ring, a pyrrole ring, an indole ring, a carbazole ring, an indolecarbazole ring, an imidazole ring, a benzimidazole ring, a pyrazole ring, an indazole ring, an oxazole ring, an isoxazole ring, a benzoxazole ring, a benzoisoxazole ring, a thiazole ring, an isothiazole ring, a benzothiazole ring, a benzoisothiazole ring, an imidazolinone ring, a benzimidazolinone ring, an imidazopyridine ring, an imidazopyrimidine ring, an imidazophenanthridine ring, a benzimidazophenanthridine ring, an azadibenzofuran ring, an azacarbazole ring, an azadibenzothiophene ring, a diazadibenzofuran ring, a diazacarbazole ring, a diazadibenzothiophene ring, a xanthone ring, a thioxanthone ring, or the like.

When the aromatic heterocyclic group includes at least two aromatic hetero rings, these rings may be bonded to each other via a single bond or condensed to each other. In addition, when the aromatic heterocyclic group includes at least two aromatic hetero rings, one atom may serve as a ring-forming atom of any of these rings.

The number of ring-forming atoms in the aromatic heterocyclic group (the sum of the number of ring-forming carbon atoms and the number of ring-forming heteroatoms) may be 3 to 30, and may be 5 to 20 in view of a peak wavelength in the emission spectrum and luminescence color purity, for example, 6 to 14. The number of ring-forming heteroatoms in the aromatic heterocyclic group may be, but is not particularly limited to, 1 to 10 in view of a peak wavelength in the emission spectrum and luminescence color purity. In addition, the number of ring-forming heteroatoms in the aromatic heterocyclic group may be 1 to 5, for example, 1 to 3. In addition, as described above, the term “ring-forming atom” refers to an atom that directly forms a ring structure.

Examples of the aromatic heterocyclic group may include, but are not particularly limited to, a thienyl group, a furanyl group, a pyrrolyl group, an imidazolyl group, a thiazolyl group, an oxazolyl group, an oxadiazolyl group, a triazolyl group, a pyridyl group, a bipyridyl group, a pyrimidyl group, a triazinyl group, an acridinyl group, a pyridazinyl group, a pyrazinyl group, a quinolinyl group, a quinazolinyl group, a quinoxalinyl group, a phenoxazinyl group, a phthalazinyl group, a pyridopyrimidinyl group, a pyridopyrazinyl group, a pyrazinopyrazinyl group, an isoquinolinyl group, an indolyl group, a carbazolyl group, a benzoxazolyl group, a benzimidazolyl group, a benzothiazolyl group, a benzocarbazolyl group, a benzothiophenyl group, a dibenzothiophenyl group, a thienothienyl group, a benzofuranyl group, a phenanthrolinyl group, a thiazolyl group, an isoxazolyl group, an oxadiazolyl group, a thiadiazolyl group, a phenothiazinyl group, a dibenzosilolyl group, a dibenzofuranyl group, a xanthonyl group, or the like. In particular, the aromatic heterocyclic group may be a triazinyl group, a carbazolyl group, a benzoxazolyl group, or a xanthonyl group.

In addition, the term “non-aromatic heterocyclic group” refers to a group derived from at least one non-aromatic hetero ring. The term “non-aromatic hetero ring” as used herein refers to a hetero ring that is partially or entirely non-aromatic. The non-aromatic hetero ring is not particularly limited, and may be, for example, a ring having at least one heteroatom (for example, a nitrogen atom (N), an oxygen atom (O), a phosphorus atom (P), a sulfur atom (S), or a silicon atom (Si)) as a ring-forming atom, wherein the remaining ring-forming atoms are carbon atoms (C). The heteroatom may be a nitrogen atom (N) or an oxygen atom (O) in view of a peak wavelength in the emission spectrum and luminescence color purity. An atom constituting a ring structure may be bonded to an exocyclic atom via a double bond. For example, a carbon atom constituting the ring structure may constitute a ketone group (C═O group), a thioketone group (C═S group), or an imine group (C═NH group), or a sulfur atom constituting the ring structure may constitute a sulfinyl group (S═O group) or a sulfonyl group (S(═O)═O group). In this case, as used herein, the exocyclic atom forming the double bond with the atom constituting the ring structure may be part of the non-aromatic hetero ring. In addition, when the exocyclic atom forming the double bond is bonded to a hydrogen atom via a single bond, the hydrogen atom may also be part of the non-aromatic hetero ring. Examples of the non-aromatic hetero ring may include, but are not particularly limited to, a pyrrolidine ring, a tetrahydrofuran ring, a tetrahydrothiophene ring, a piperidine ring, a tetrahydropyran ring, a tetrahydrothiopyran ring, a dioxane ring, a morpholine ring, a dioxolane ring, or the like.

When the non-aromatic heterocyclic group includes at least two non-aromatic hetero rings, these rings may be bonded to each other via a single bond or condensed to each other. In addition, when the non-aromatic heterocyclic group includes at least two non-aromatic hetero rings, one atom may serve as a ring-forming atom of any of these rings.

The number of ring-forming atoms in the non-aromatic heterocyclic group (the sum of the number of ring-forming carbon atoms and the number of ring-forming heteroatoms) may be 3 to 30, and may be 5 to 20 in view of a peak wavelength in the emission spectrum and luminescence color purity, for example, 6 to 14. The number of ring-forming heteroatoms in the non-aromatic heterocyclic group may be, but is not particularly limited to, 1 to 10 in view of a peak wavelength in the emission spectrum and luminescence color purity. In the same point of view, the number of ring-forming heteroatoms in the non-aromatic heterocyclic group may be 1 to 5, for example, 1 to 3. In addition, as described above, the term “ring-forming atom” refers to an atom that directly forms a ring structure. Accordingly, when there is an exocyclic atom forming a double bond with an atom constituting a ring structure, the exocyclic atom may not be included in the ring-forming atom.

Examples of the non-aromatic heterocyclic group may include, but are not particularly limited to, a pyrrolidinyl group, a tetrahydrofuranyl group, a tetrahydrothienyl group, a piperidinyl group, a tetrahydropyranyl group, a tetrahydrothiopyranyl group, a dioxanyl group, a morphonyl group, a dioxolanyl group, or the like.

In addition, the term “substituted heterocyclic group” refers to a group formed by substituting an unsubstituted heterocyclic group with a substituent. Therefore, when the number of ring-forming atoms in the heterocyclic group is equal to or less than a certain upper limit of the number of ring-forming atoms, for example, 30 or less, and the substituent forms a ring structure, the number of ring-forming atoms in the substituted heterocyclic group may be greater than the upper limit.

In the case of the substituted or unsubstituted saturated hydrocarbon group or saturated heterocyclic group, each formed by bonding two adjacent groups among R¹ to R¹⁶ and having 5 to 9 ring-forming atoms, of (a10), two adjacent groups (for example, R¹ and R²; R² and R³; or R³ and R⁴) among R¹ to R¹⁶, which are each bonded to ring-forming carbon atom of a benzene ring (among four benzene rings other than the central benzene ring) located on the outer side of the compound of Formula (1), are each bonded to a ring-forming carbon atom of a benzene ring via a single bond and are bonded to each other via a single bond. In other words, in the case of the group of (a10), two adjacent groups among R¹ to R¹⁶ are bonded to each other to form a saturated hydrocarbon group or a saturated heterocyclic group, each condensed with a benzene ring and having 5 to 9 ring-forming atoms. Positions of condensed rings (positions of condensation in the benzene ring), which are formed by two adjacent groups among R¹ to R¹⁶, may be at the position 2 and position 3 of the benzene ring, and accordingly, R² and R³; R⁶ and R⁷; R¹⁰ and R¹¹; or R¹⁴ and R¹⁵ may be bonded to each other to form a saturated hydrocarbon group or a saturated heterocyclic group, each having 5 to 9 ring-forming atoms. In an embodiment, R² and R³; and R¹⁰ and R¹¹ may be bonded to each other to form a saturated hydrocarbon group or a saturated heterocyclic group, each having 5 to 9 ring-forming atoms, or R⁶ and R⁷; and R¹⁴ and R¹⁵ may be bonded to each other to form a saturated hydrocarbon group or a saturated heterocyclic group, each having 5 to 9 ring-forming atoms. In other words, when a condensed ring with a benzene ring is formed by R¹ to R¹⁶, the number of condensed rings may be 2.

A condensed ring (a saturated hydrocarbon ring) formed by the saturated hydrocarbon group having 5 to 9 ring-forming atoms may include, for example, a cyclopentane ring, a cyclohexane ring, a cycloheptane ring, a cyclooctane ring, a cyclononane ring, or the like. A condensed ring (a saturated hetero ring) formed by the saturated heterocyclic group having 5 to 9 ring-forming atoms may include a group having 5 to 9 ring-forming atom among the non-aromatic heterocyclic groups. Examples of the condensed ring (the saturated hetero ring) formed by the saturated heterocyclic group having 5 to 9 ring-forming atoms may include a pyrrolidine ring, a tetrahydrofuran ring, a tetrahydrothiophene ring, a piperidine ring, a tetrahydropyran ring, a tetrahydrothiopyran ring, a dioxane ring, a morpholine ring, a dioxolane ring, or the like.

In addition, the term “substituted saturated hydrocarbon group or saturated heterocyclic group having 5 to 9 ring-forming atoms” refers to a group formed by substituting an unsubstituted saturated hydrocarbon group or saturated heterocyclic group with a substituent. Therefore, when the number of ring-forming atoms in the saturated hydrocarbon group or the saturated heterocyclic group is equal to or less than a certain upper limit of the number of ring-forming atoms, for example, 9 or less, and the substituent forms a ring structure, the number of ring-forming atoms in the substituted saturated hydrocarbon group or saturated heterocyclic group may be greater than the upper limit.

A halogen atom, a cyano group, an unsubstituted alkyl group having 1 to 20 carbon atoms, an unsubstituted alkoxy group having 1 to 20 carbon atoms, an unsubstituted arylamino group having 6 to 20 carbon atoms, an unsubstituted aromatic hydrocarbon group having 6 to 30 carbon atoms, and an unsubstituted heterocyclic group having 3 to 30 ring-forming atoms, which are substituents that are substituted into the groups of (a4) to (a10), may each be the same as the unsubstituted groups in the description of (a2) to (a10).

Examples of the unsubstituted haloalkyl group having 1 to 20 carbon atoms, which is a substituent that is substituted into the groups of (a4) to (a10), may include, among the alkyl groups described in (a4), a group in which at least one hydrogen atom is substituted by the halogen atom described in (a2). The halogen atom may be a fluorine atom in view of luminescence efficiency. Examples of the haloalkyl group may include a trifluoromethyl group, a trichloromethyl group, a tribromomethyl group, a triiodomethyl group, or the like. In particular, the haloalkyl group may be a fluorinated alkyl group, or for example, a trifluoromethyl group.

In the unsubstituted alkylamino group having 1 to 20 carbon atoms, which is a substituent that substitutes the groups of (a4) to (a10), a nitrogen atom of the unsubstituted alkylamino group is bonded to any one atom constituting the unsubstituted groups of (a4) to (a10) in Formula (1) via a single bond. An alkyl group substituting the alkylamino group is not particularly limited, but may be the same as the description of (a3). The alkylamino group may be, but is not particularly limited to, a monoalkylamino group or a dialkylamino group. Examples of the alkylamino group may include, but are not particularly limited to, a N-methylamino group, a N-ethylamino group, a N-propylamino group, a N-isopropylamino group, a N-butylamino group, a N-isobutylamino group, a N-sec-butylamino group, a N-tert-butylamino group, a N-pentylamino group, a N-hexylamino group, a N,N-dimethylamino group, a N-methyl-N-ethylamino group, a N,N-diethylamino group, a N,N-dipropylamino group, a N,N-diisopropylamino group, a N,N-dibutylamino group, a N,N-diisobutylamino group, a N,N-dipentylamino group, and a N,N-dihexylamino group.

In this regard, a substituent that is substituted into the groups of (a4) to (a10) may be, for example, a halogen atom, a cyano group, an unsubstituted alkyl group having 1 to 20 carbon atoms, an unsubstituted alkoxy group having 1 to 20 carbon atoms, or an unsubstituted arylamino group having 6 to 20 carbon atoms. In addition, in particular, the substituent may be, for example, a halogen atom, an unsubstituted alkyl group having 1 to 20 carbon atoms, or for example, a fluorine atom or an unsubstituted linear or branched alkyl group having 1 to 20 carbon atoms. For example, the substituent may be a fluorine atom, a methyl group, an ethyl group, an iso-propyl group, or a tert-butyl group.

In an embodiment, a substituent that is substituted into the groups of (a4) to (a10) may be an unsubstituted aromatic hydrocarbon group having 6 to 30 carbon atoms. In particular, the substituent may be a group derived from a benzene ring.

In an embodiment, a substituent that substitutes the groups of (a4) to (a10) may be an unsubstituted heterocyclic group having 3 to 30 ring-forming atoms. In particular, a heteroatom may be a heterocyclic group including an oxygen or nitrogen atom, for example, a dibenzofuranyl group, a carbazolyl group, or a benzoxazolyl group. In addition, the heteroatom may be a dibenzofuranyl group or a carbazolyl group.

A substituent that is substituted into the group of (a4) may be a halogen atom or an unsubstituted alkyl group having 1 to 20 carbon atoms. Therefore, a substituent for the “substituted alkyl group having 1 to 20 carbon atoms” may be a halogen atom or an unsubstituted alkyl group having 1 to 20 carbon atoms. In particular, the substituent may be an unsubstituted linear or branched alkyl group having 1 to 20 carbon atoms. In addition, the substituent may be a methyl group, an ethyl group, an iso-propyl group, or a t-butyl group.

A substituent that is substituted into the group of (a7) may be a halogen atom or an unsubstituted alkyl group having 1 to 20 carbon atoms. Therefore, a substituent for the “substituted triarylsilyl group, alkyldiarylsilyl group, dialkylarylsilyl group, or trialkylsilyl group” may be a halogen atom or an unsubstituted alkyl group having 1 to 20 carbon atoms. In particular, the substituent may be an unsubstituted linear or branched alkyl group having 1 to 20 carbon atoms. For example, the substituent may be a methyl group, an ethyl group, an iso-propyl group, or a t-butyl group.

A substituent that is substituted into the group of (a10) may be a halogen atom or an unsubstituted alkyl group having 1 to 20 carbon atoms. Therefore, a substituent for the “substituted saturated hydrocarbon group or saturated heterocyclic group, each formed by bonding two adjacent groups among R¹ to R¹⁶ and having 5 to 9 ring-forming atoms,” may be a halogen atom or an unsubstituted alkyl group having 1 to 20 carbon atoms. In particular, the substituent may be an unsubstituted linear or branched alkyl group having 1 to 20 carbon atoms. For example, the substituent may be a methyl group, an ethyl group, an iso-propyl group, or a t-butyl group.

When the groups of (a4) to (a10) are substituted groups, the substituent that are substituted into the groups of (a4) to (a10) may be further substituted with an additional substituent. The additional substituent is not particularly limited, but may be, for example, a substituent in a case where the groups of (a4) to (a10) are substituted groups, or one of these groups substituted by these groups.

In an embodiment, R¹ to R¹⁶ in the compound of Formula (1) are groups other than the substituent W and may include at least one selected from the following Group X. In other words, in an embodiment, at least one (for example, at least two) of R¹ to R¹⁶ in the compound of Formula (1) may be the substituent W, and at least one (for example, at least two) of R¹ to R¹⁶ may be a group selected from the following Group X. In addition, in the compound of Formula (1), at least one of R¹ to R⁴ and at least one of R⁹ to R¹² may each be the substituent W, and at least one of R⁵ to R⁸ and at least one of R¹³ to R¹⁶ may each be a group selected from the following Group X.

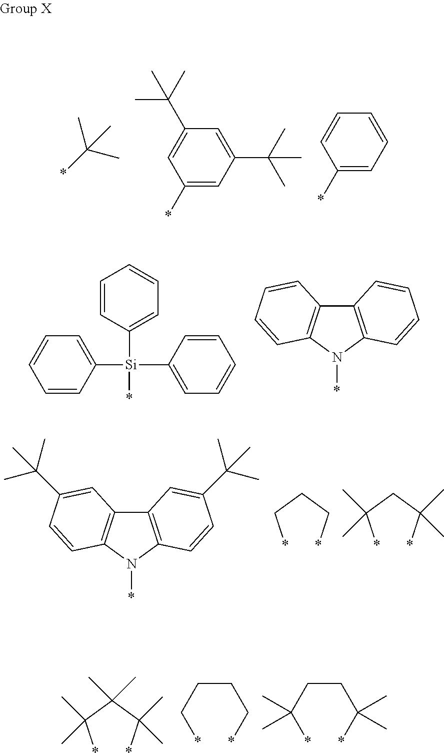

Among the above substituents, a substituent having two binding sites (*) with a benzene ring is bonded to two adjacent positions among R¹ to R¹⁶ and are condensed with a benzene ring to form a condensed ring. For example, the following group of (a-1) is condensed with a benzene ring to form a 5-membered ring (a-2), and the following group of (b-1) is condensed with a benzene ring to form a 6-membered ring (b-2).

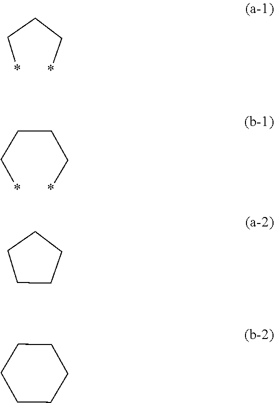

In addition, in an embodiment, R¹ to R¹⁶ in the compound of Formula (1) are groups other than the substituent W and may include at least one selected from the following Group Y. In other words, in an embodiment, at least one (for example, at least two) of R¹ to R¹⁶ in the compound of Formula (1) may be the substituent W, and at least one (for example, at least two) of R¹ to R¹⁶ may be a group selected from the following Group Y. In addition, in the compound of Formula (1), at least one of R¹ to R⁴ and at least one of R⁹ to R¹² may each be the substituent W, and at least one of R⁵ to R⁸ and at least one of R¹³ to R¹⁶ may each be a group selected from the following Group Y.

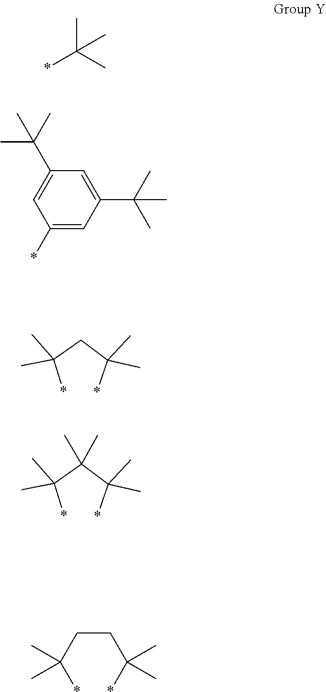

Hereinafter, the compound of Formula (1) according to an embodiment is described in detail. However, the disclosure is not limited to such examples. For example, examples of the compound of Formula (1) according to an embodiment may be represented by one of Compounds (100) to (117).

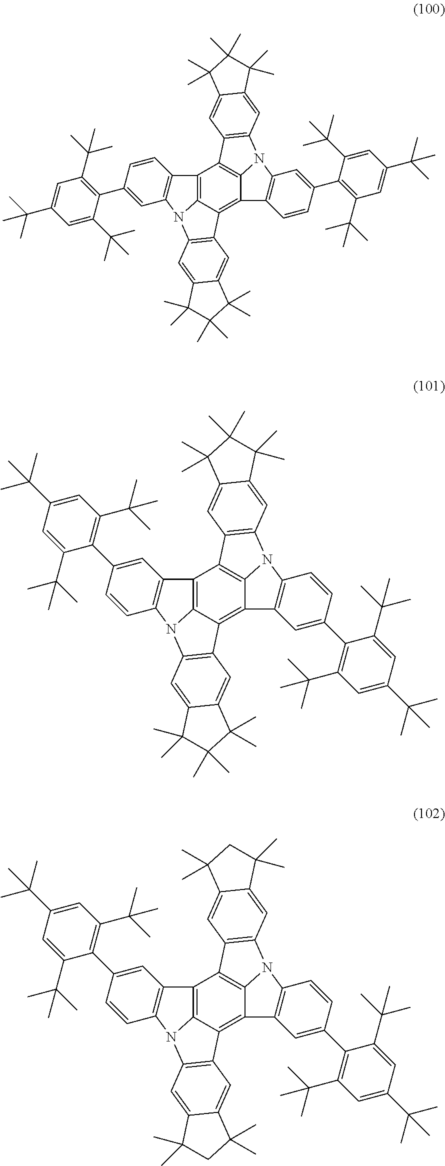

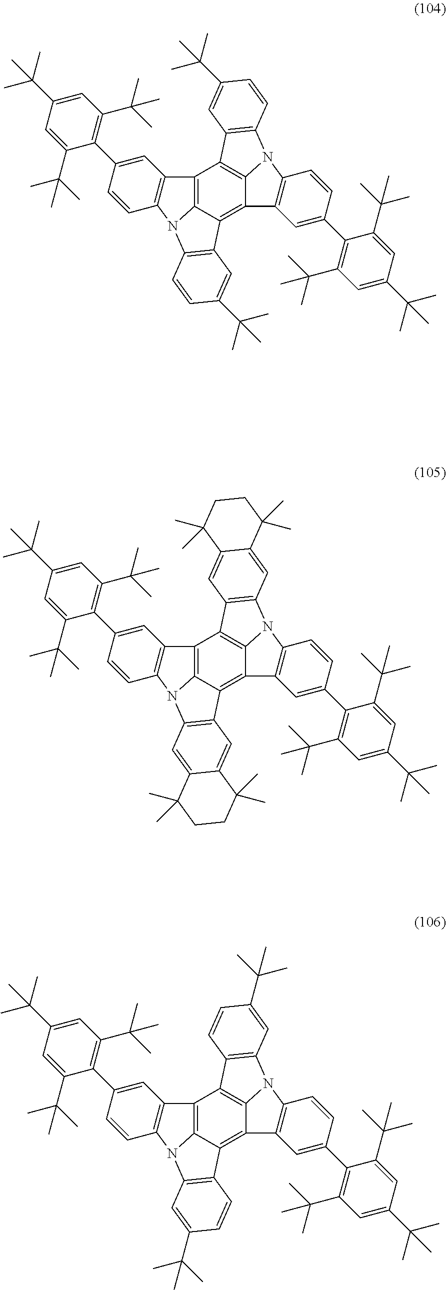

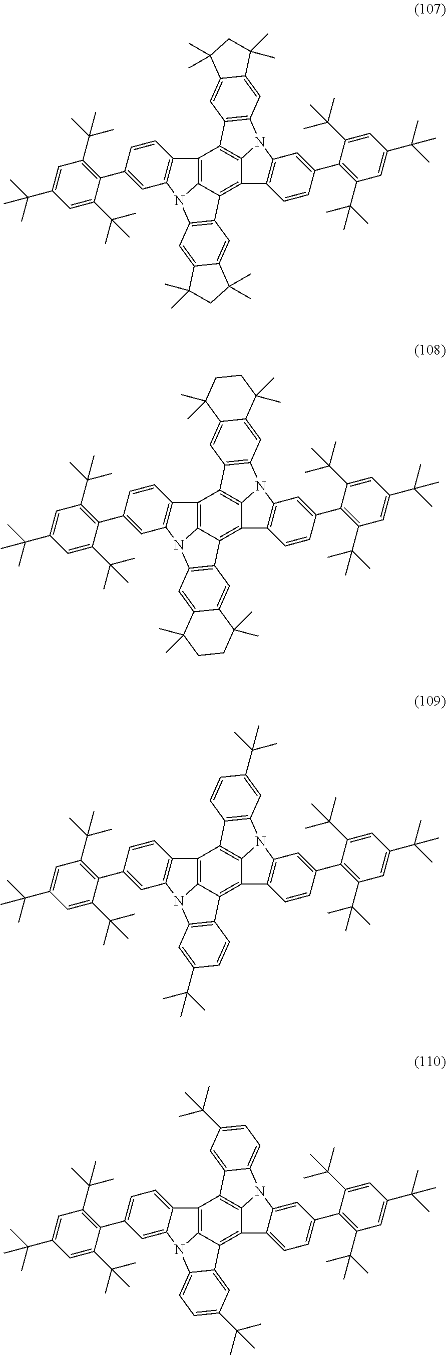

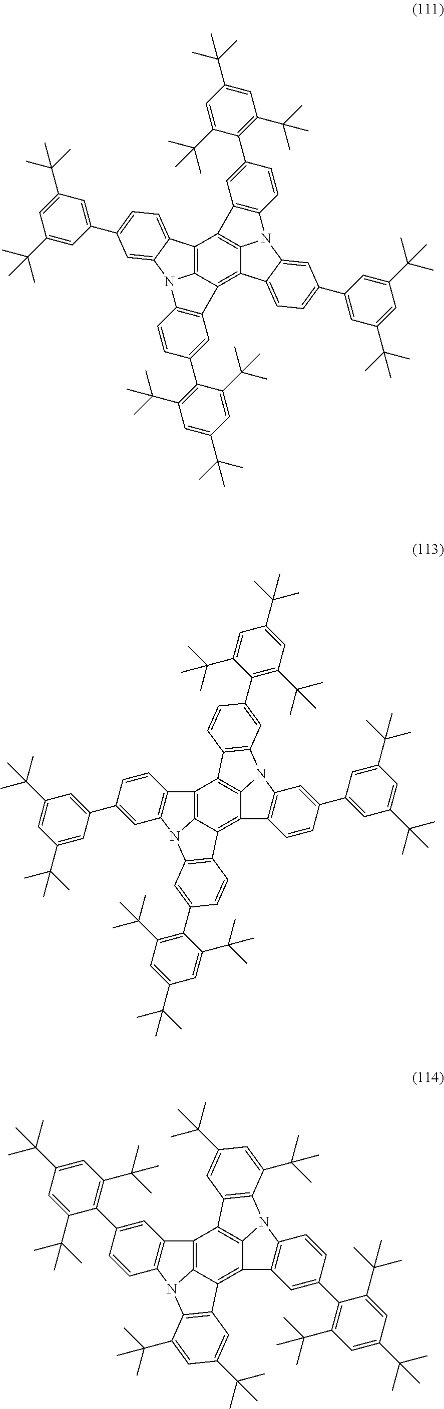

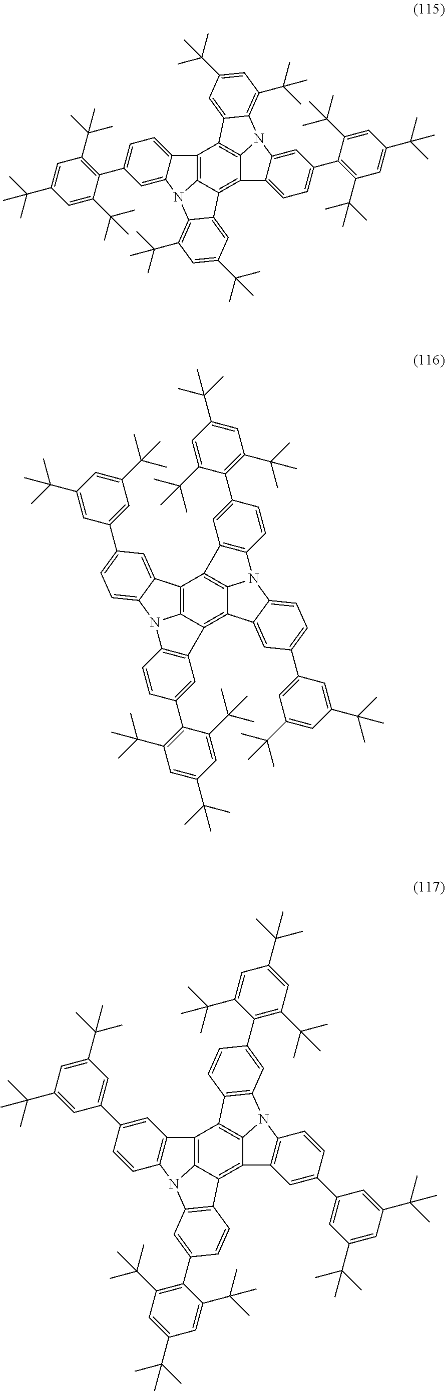

For example, examples of the compound of Formula (1) may include Compounds 101, 102, 105, 108, 110, 111, 113, etc.

The compound of Formula (1) according to the disclosure may have a peak wavelength in the emission spectrum within a blue wavelength region and may provide luminescence with high color purity. In addition, the blue wavelength region as used herein refers to a wavelength region of at least about 380 nanometers (nm) and not more than about 500 nm. A peak wavelength of emission of photoluminescence (PL) of the compound of Formula (1) according to the disclosure is not particularly limited, but may be about 440 nm to about 480 nm. In addition, the peak wavelength may be of light having a peak in a wavelength region of about 445 nm to about 470 nm, for example, about 450 nm to about 470 nm, for example, about 450 nm to about 465 nm. When the peak wavelength is within the above range, excellent luminescence, in particular, blue luminescence, may be obtained. The FWHM of a peak of an emission spectrum of PL may be 30 nm or less, for example, 20 nm or less, for example, 15 nm or less (the lower limit is greater than 0 nm). In addition, the peak wavelength of emission of the PL and the FWHM of the peak of the emission spectrum of the PL may be measured by using a spectrofluorometer F7000 manufactured by Hitachi High-Tech Co., Ltd. More specifically, by measurement at room temperature at an excitation wavelength of 360 nm, such a spectrofluorometer may be used to evaluate 1×10⁻⁵ M (moles per liter (mol/L), moles per cubic decimeter, (mol/dm³)) of a toluene solution of the compound of Formula (1) according to the disclosure or a thin film formed by depositing the compound of Formula (1) according to the disclosure and a host molecule by using a method described in the following Examples.

The narrow FWHM and full width at quarter maximum (FWQM), thermal activated delayed fluorescence (TADF) characteristics and emission wavelength required for a molecule used as a dopant may be predicted using a quantum chemical calculation.

A method of synthesizing the compound of Formula (1) according to the disclosure is not particularly limited, and the synthesis may be performed using known synthesis methods. More specifically, the synthesis may be performed according to or in view of the method described in the Examples. For example, the synthesis may be performed by changing the raw material or the reaction condition in the method described in the Examples, adding or deleting some processes, or appropriately combining known synthesis methods.

A method of identifying the structure of the compound of Formula (1) according to the disclosure is not particularly limited. The structure of the compound of Formula (1) according to the disclosure may be identified by, for example, a known method (for example, nuclear magnetic resonance spectroscopy (NMR), liquid chromatography-mass spectrometry (LC-MS), etc.).

### Material for Organic EL Device

Another aspect of the disclosure relates to a material for an organic electroluminescent device, the material including the compound of Formula (1). The material may be a material for an emission layer.

The material for an organic electroluminescent device according to an embodiment may include the compound of Formula (1) and other materials used in an organic electroluminescent device. The other materials used in an organic electroluminescent device are not particularly limited, but may be phosphorescent compounds or host materials. In addition, the other materials may be a phosphorescent complex and a host material. In this regard, the compound of Formula (1) may be used as a dopant material, and the phosphorescent complex may be used as an auxiliary dopant material. Luminescence efficiency may be significantly improved by using both the compound of Formula (1) and the phosphorescent complex or the host material (for example, the phosphorescent complex and the host material). Not wishing to be bound by theory, it is believed that when the material for an organic electroluminescent device contains the host material, the phosphorescent complex receives energy from the host material. In addition, the phosphorescent complex is assumed to transfer energy to the compound of Formula (1) by a fluorescence resonance energy transfer (FRET) mechanism. As a result, highly efficient energy transfer may occur from the phosphorescent complex to the compound of Formula (1). In addition, the other materials used in an organic electroluminescent device may include other materials known in the art.

The amount of the compound of Formula (1) relative to the total weight of the material for an organic electroluminescent device (in particular, the material for an emission layer) is not particularly limited, but may be 0.05 weight percent (wt %) or more. In addition, the amount of the compound of Formula (1) may be 0.1 wt % or more, for example, 0.2 wt % or more. Within this range, an organic electroluminescent device having excellent luminescence color purity and high luminescence efficiency may be obtained. The amount of the compound of Formula (1) relative to the total weight of the material for an organic electroluminescent device may be 50 wt % or less. In addition, the amount of the compound of Formula (1) may be 30 wt % or less, for example, 25 wt % or less. Within this range, an organic electroluminescent device having excellent luminescence color purity and high luminescence efficiency may be obtained. In addition, in an emission layer of an organic electroluminescent device described later, the amount of the compound of Formula (1) relative to the total weight of the emission layer may be the same as described above.

### Phosphorescent Complex

The material for an organic electroluminescent device according to an embodiment may further include a phosphorescent complex in addition to the compound of Formula (1). Luminescence efficiency may be significantly improved by including the phosphorescent complex. Luminescence efficiency may be significantly improved by using both the compound of Formula (1) and the phosphorescent complex. The reason for the improved performance is assumed to be due to transfer of energy to the compound of Formula (1) by the phosphorescent complex via a FRET mechanism. As a result, highly efficient energy transfer may occur from the phosphorescent complex to the compound of Formula (1). It is assumed that the above effect is achieved because highly efficient energy transfer is possible from the phosphorescent complex to the compound of Formula (1).

The phosphorescent complex is not particularly limited, but may be a metal complex in view of luminescence efficiency. In the same point of view, the phosphorescent complex may be a platinum complex or a palladium complex, for example, a platinum complex. Therefore, in the material for an organic electroluminescent device according to an embodiment, for example, the phosphorescent complex may be a platinum complex.

The phosphorescent complex is not particularly limited, but may be, for example, a compound having the structure of Formula (4) in view of luminescence color purity and luminescence efficiency.

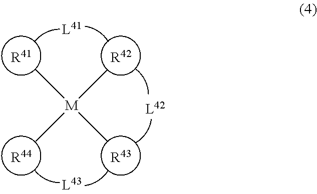

M in Formula (4) may be a metal ion having a coordination number of 4,
R⁴¹, R⁴², R⁴³, and R⁴⁴ may each independently be a substituted or unsubstituted hydrocarbon cyclic group or a substituted or unsubstituted heterocyclic group,
L⁴¹ may be a linking group linking R⁴¹ and R⁴²,
L⁴² may be a linking group linking R⁴² and R⁴³, and
L⁴³ may be a linking group linking R⁴³ and R⁴⁴.

The hydrocarbon cyclic group In Formula (4) refers to a group derived from at least one hydrocarbon ring. When the hydrocarbon cyclic group includes at least two hydrocarbon rings, some or all of these rings may be bonded to or condensed with each other via a single bond. In addition, when the hydrocarbon cyclic group includes at least two hydrocarbon rings, one atom may serve as a ring-forming atom of any of these rings.

The heterocyclic group in Formula (4) is as described for the monovalent heterocyclic group of the group of (a9) in Formula (1), except that the valency thereof may be different.

A substituent that is substituted into the hydrocarbon cyclic group or the heterocyclic group in Formula (4) is not particularly limited, but may be a substituent that is substituted into the groups of (a4) to (a10) in Formula (1).

M in Formula (4) may be, for example, a platinum (Pt) ion or a palladium (Pd) ion, for example, a platinum (Pt) ion.

A known compound may be used as the phosphorescent complex. For example, the platinum complex described in Tyler Fleetham et al., “Efficient “Pure” Blue OLEDs Employing Tetradentate Pt Complexes with a Narrow Spectral Bandwidth,” Advanced Materials, 2014, 26, 7116-7121, the platinum complex described in European Patent Publication No. EP 3670520, the platinum complex and palladium complex described in Japanese Patent Publication No. JP 2019-029500, or the platinum complex described in U.S. Patent Publication No. US 2015/0162552 may be used.

Hereinafter, the phosphorescent complex according to an embodiment is described in detail. However, the disclosure is not limited to such examples. Examples of the phosphorescent complex may include compound P1 to P120, or a combination thereof.

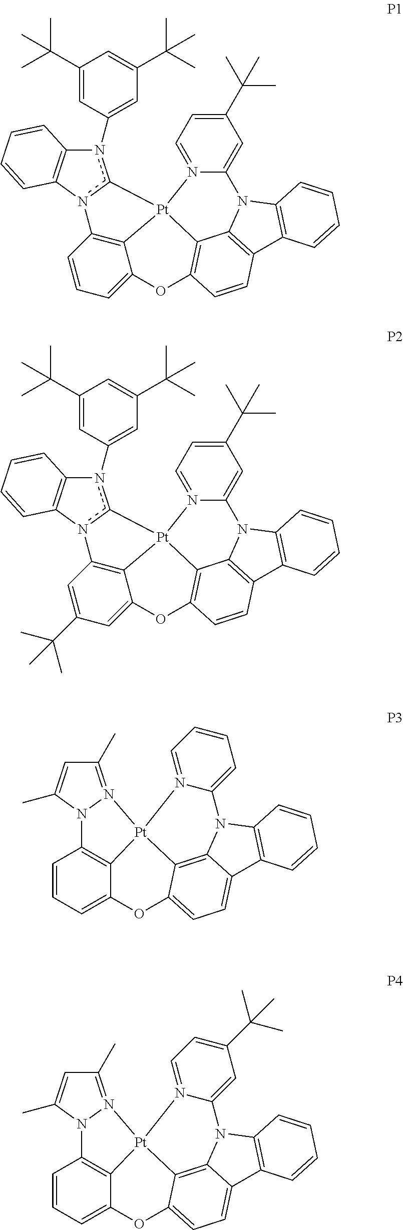

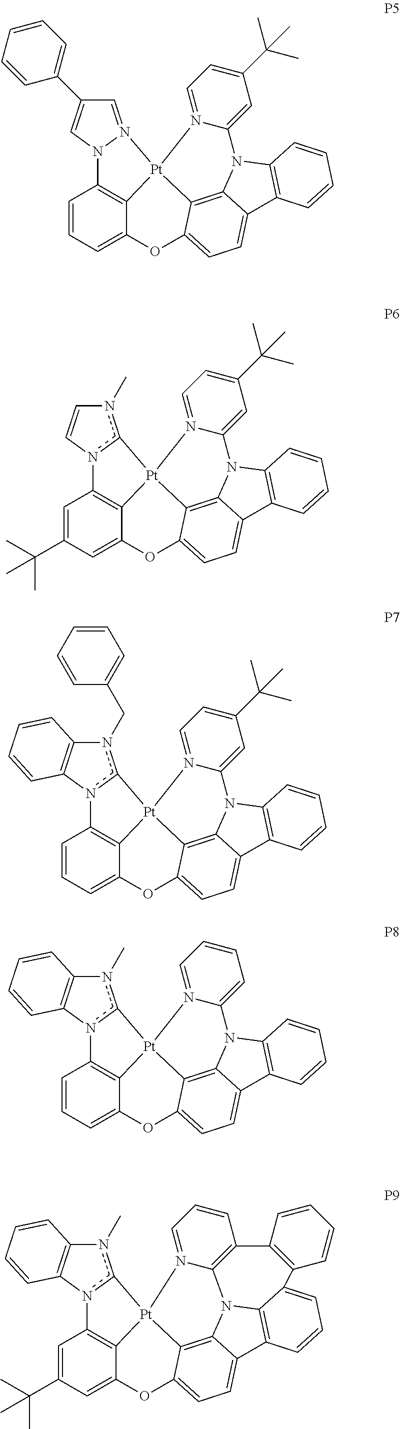

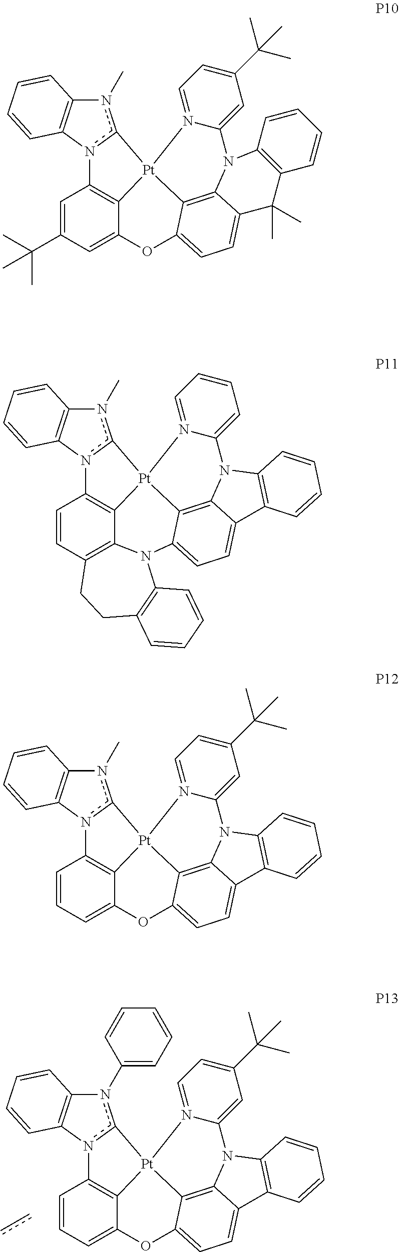

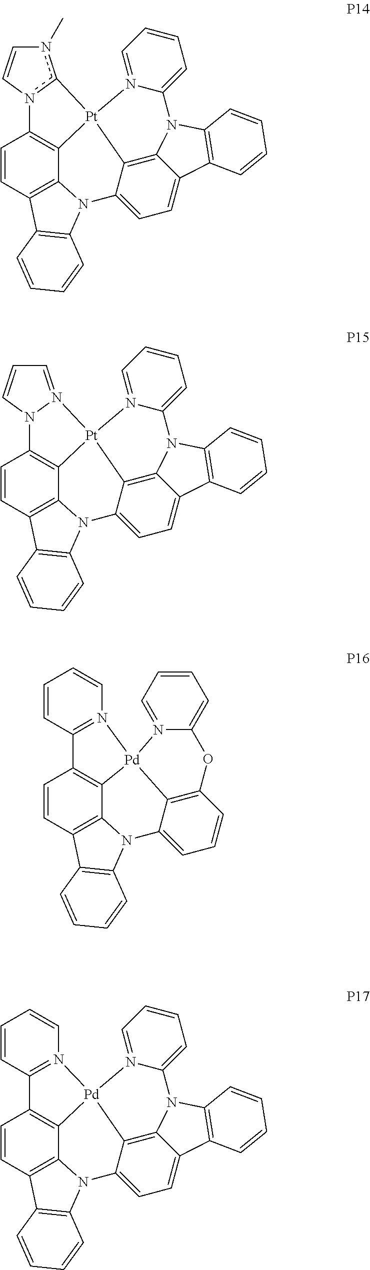

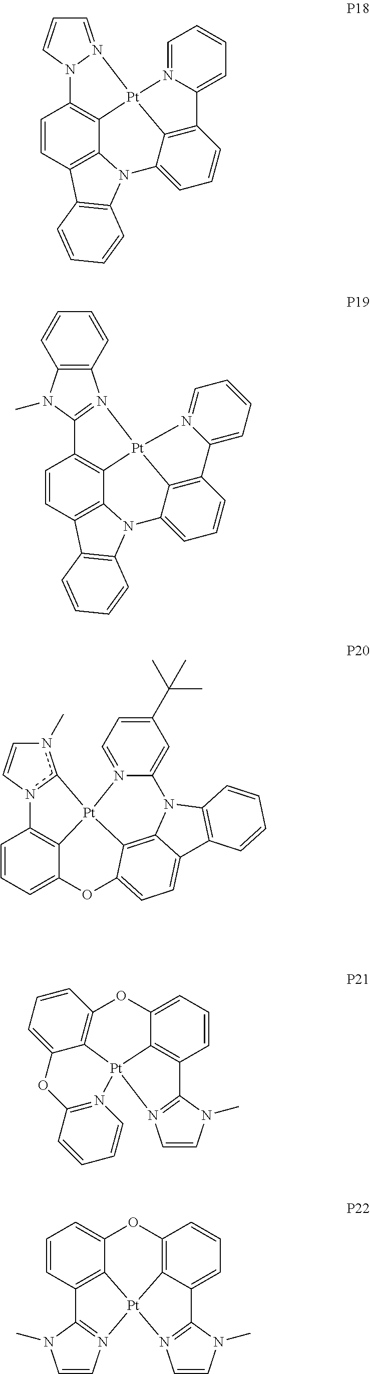

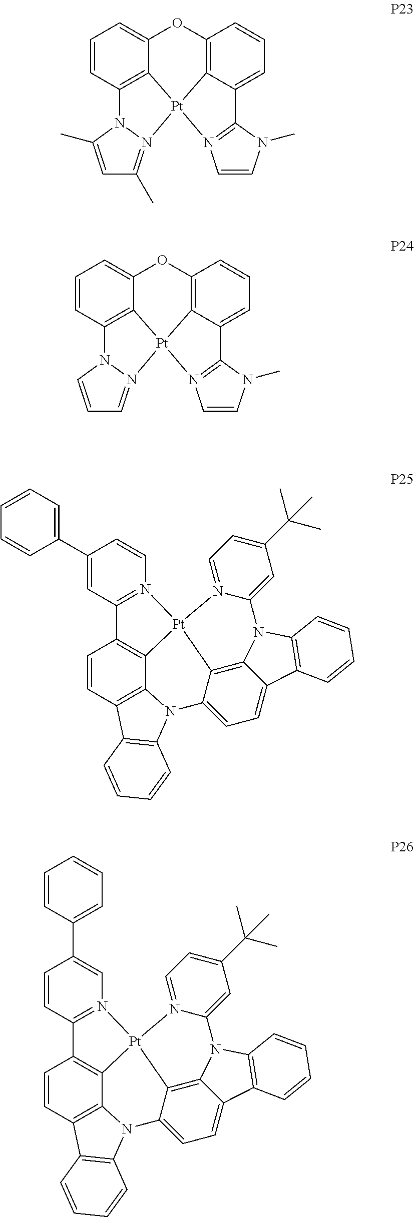

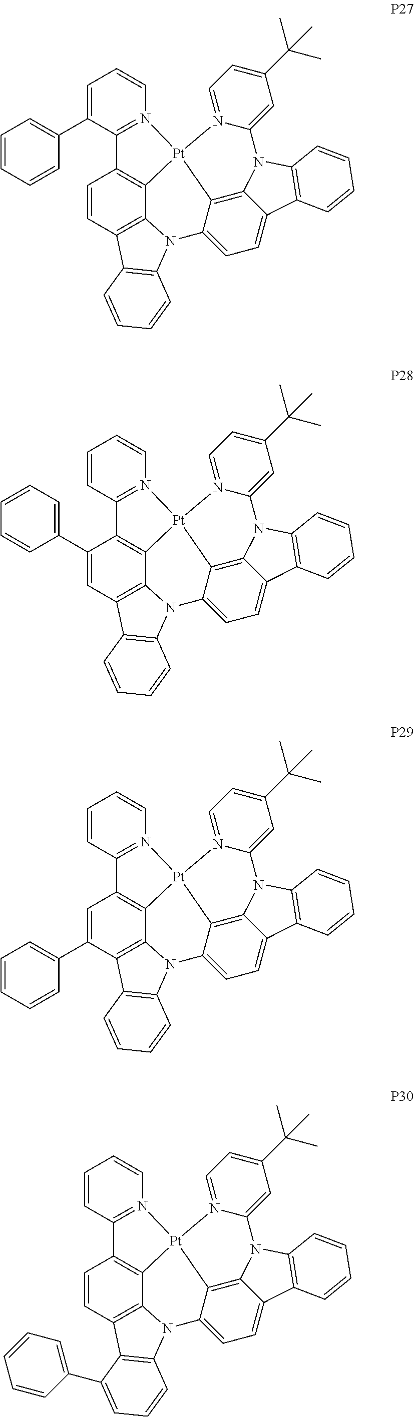

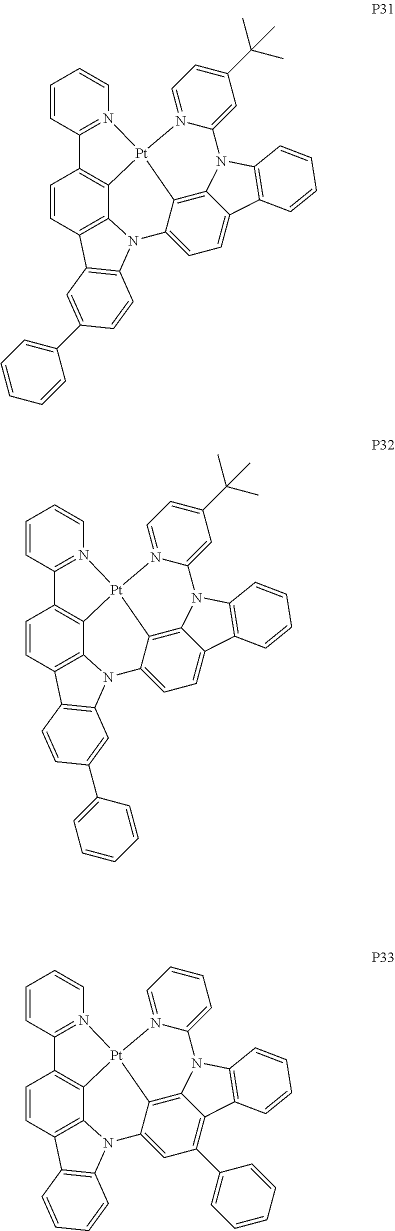

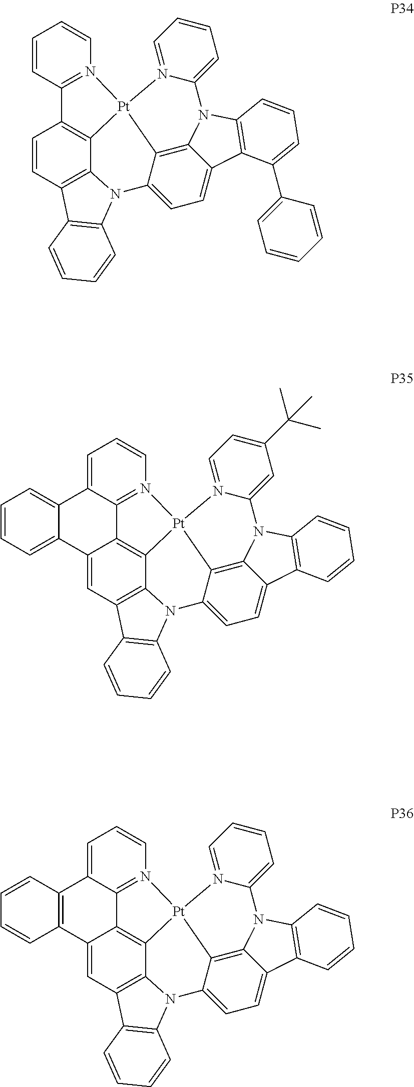

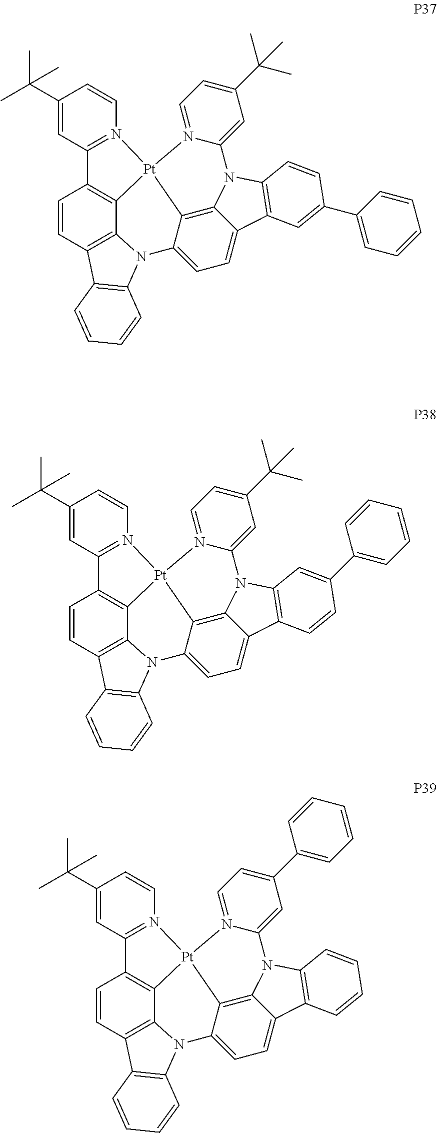

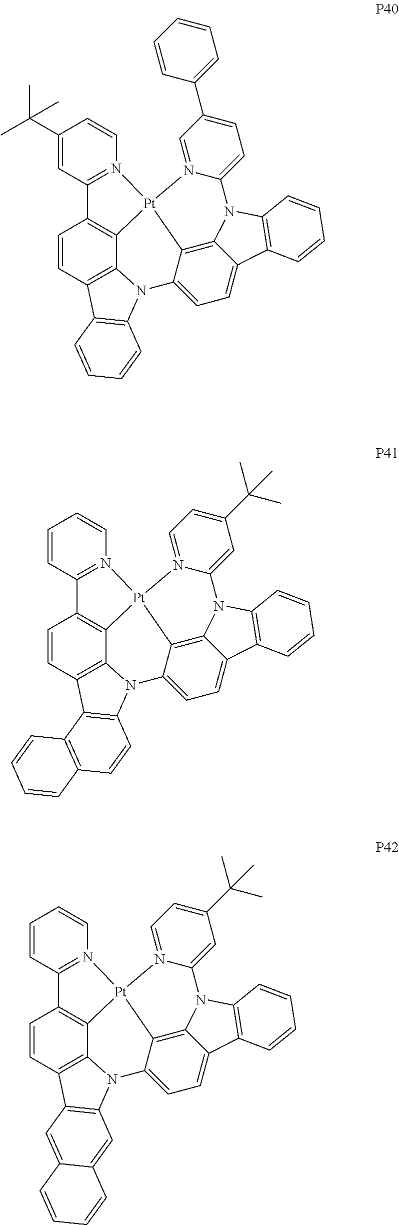

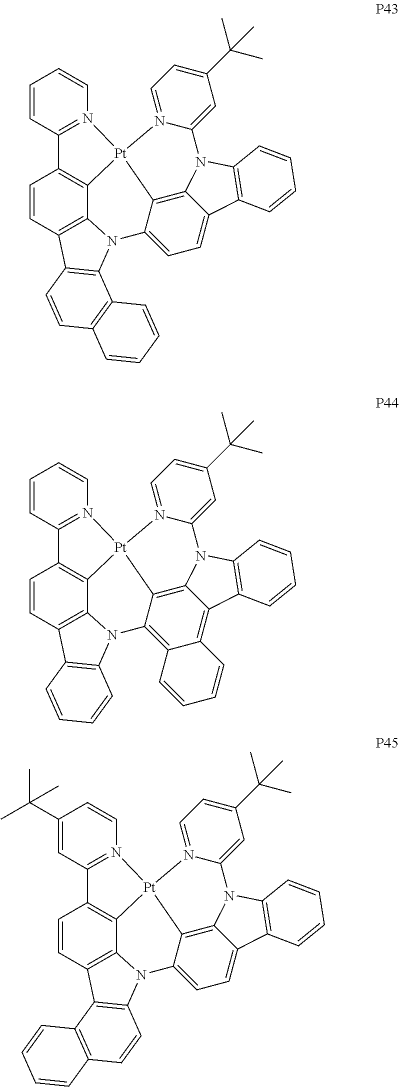

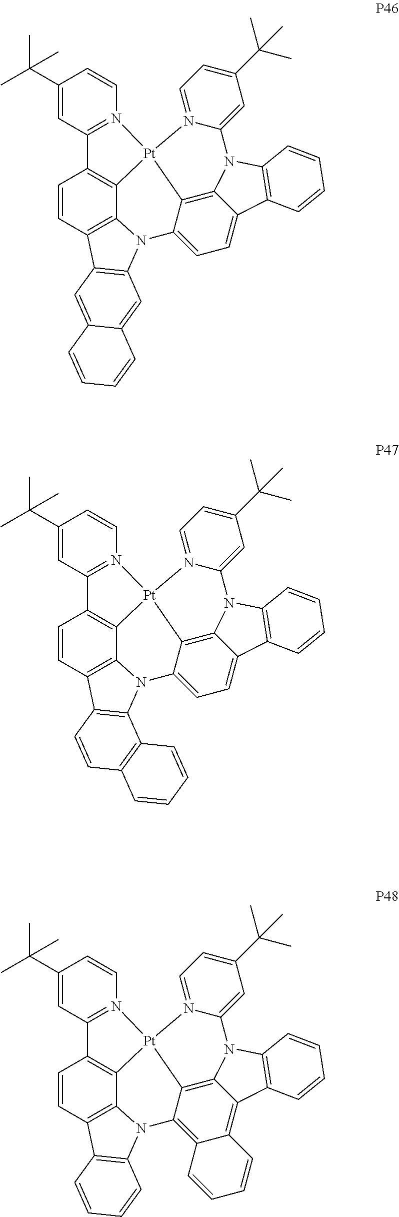

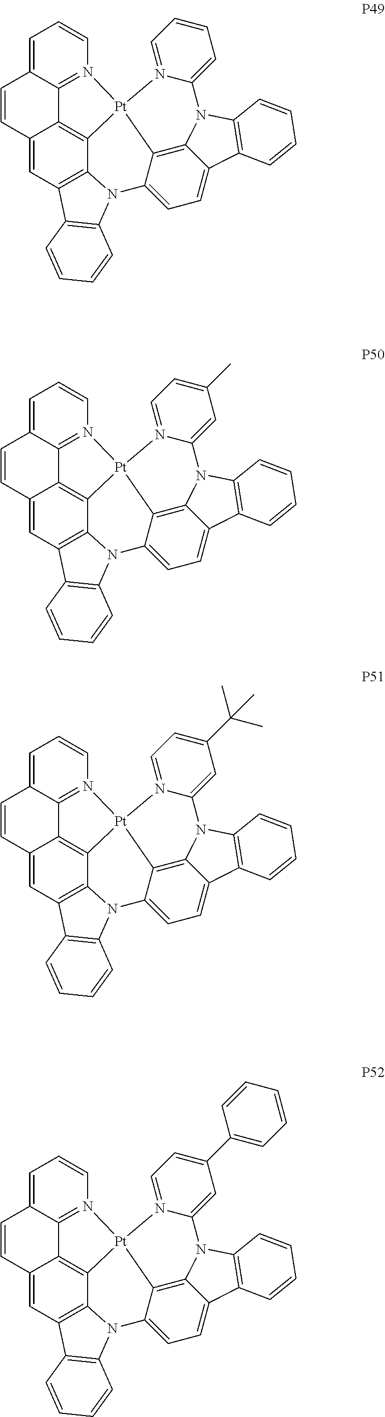

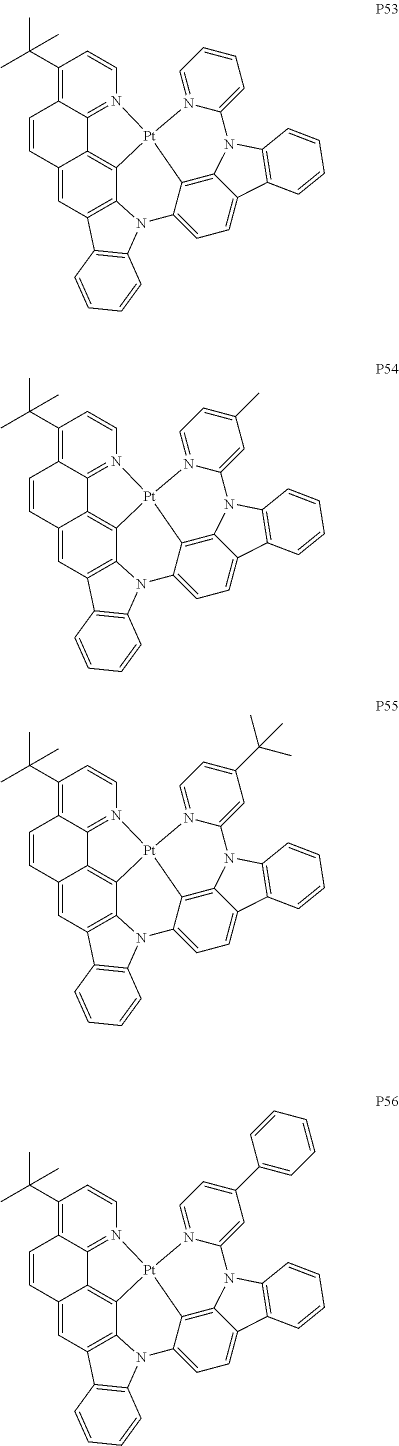

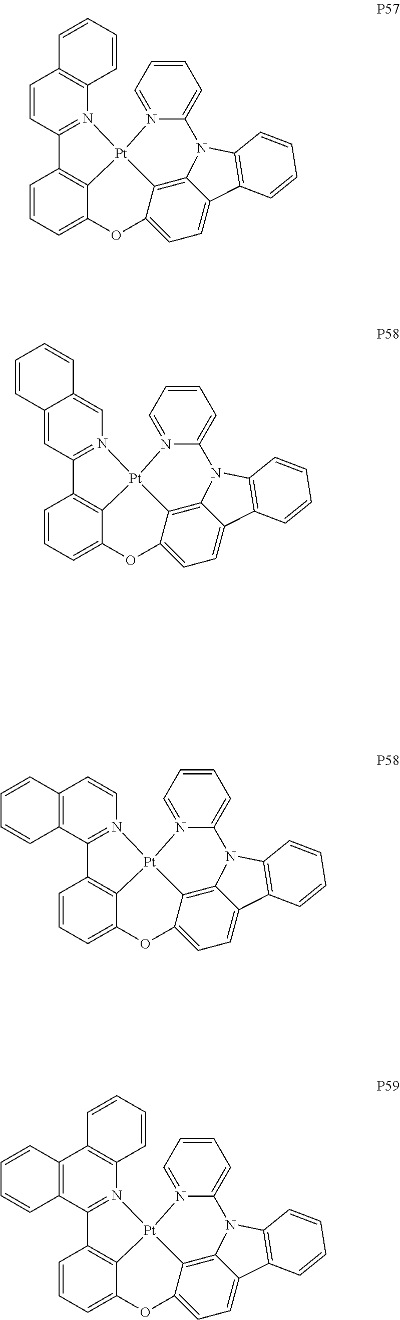

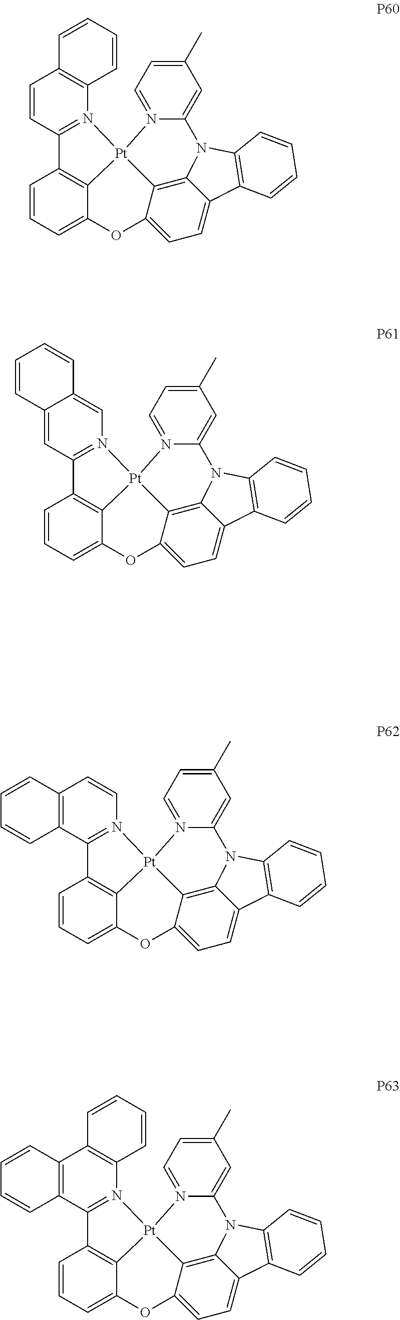

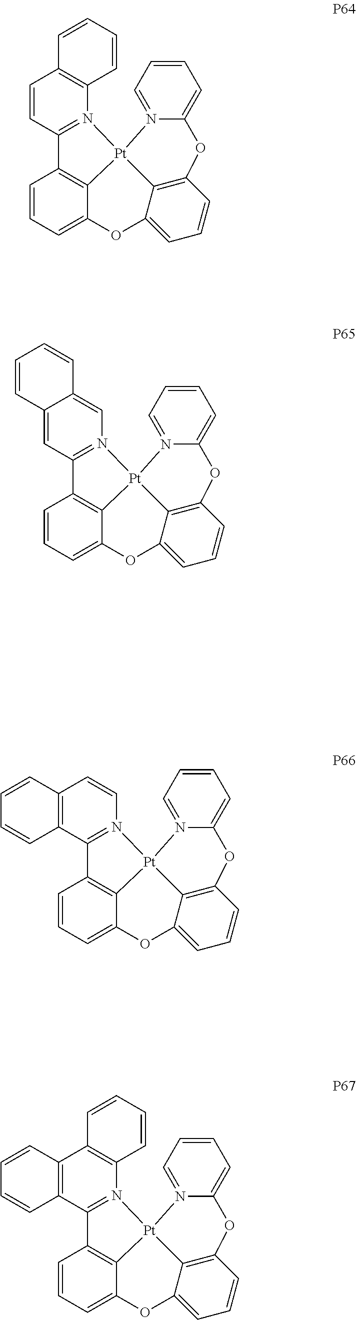

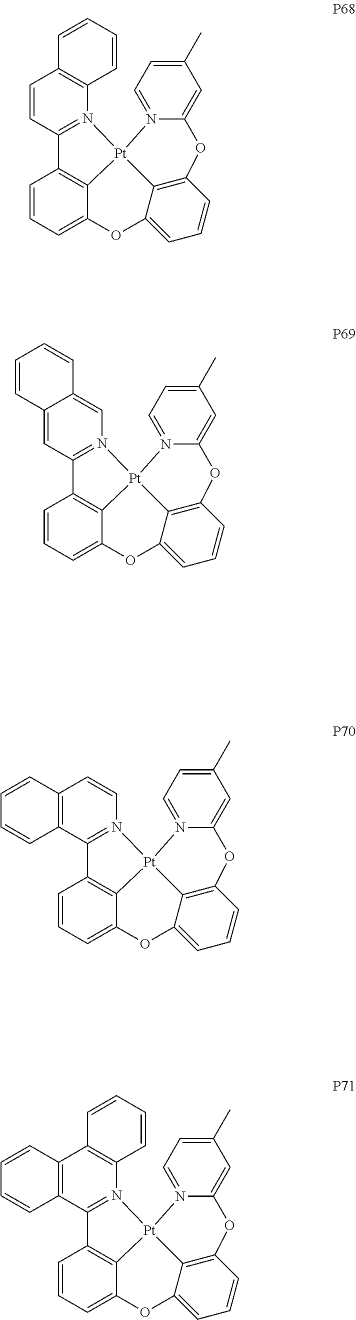

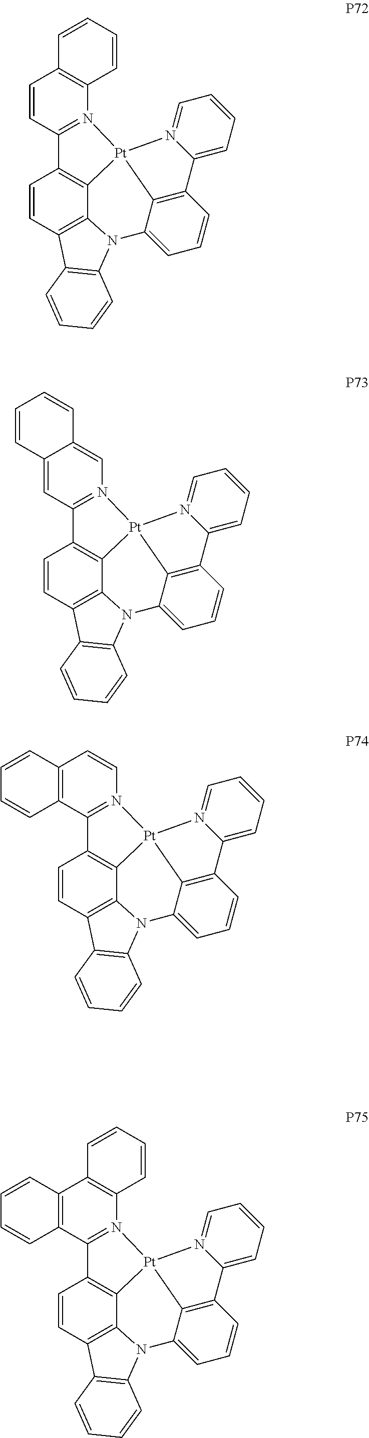

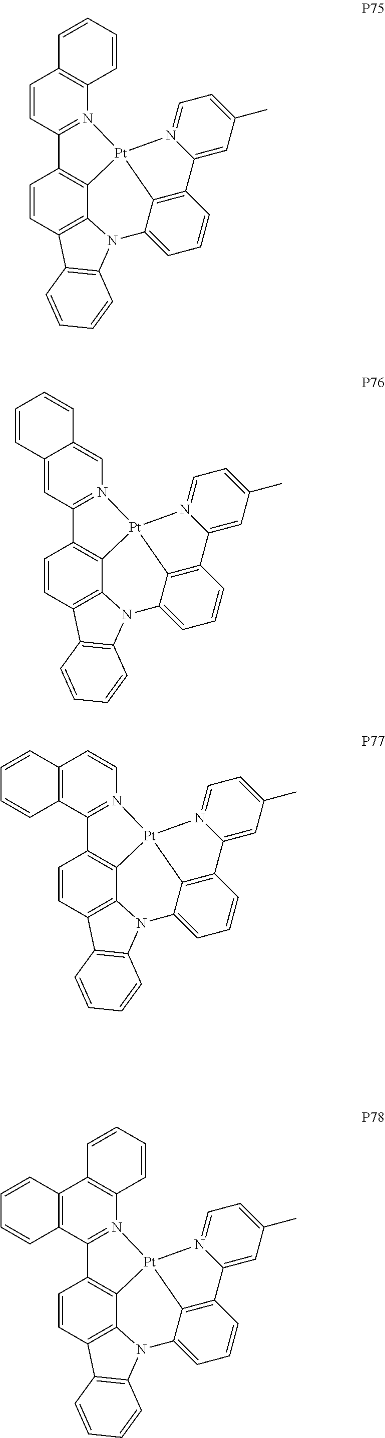

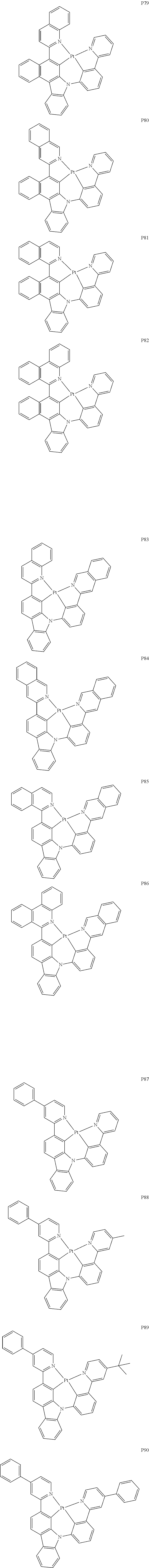

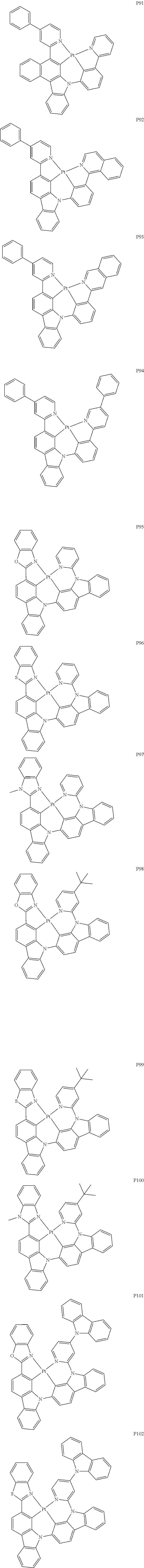

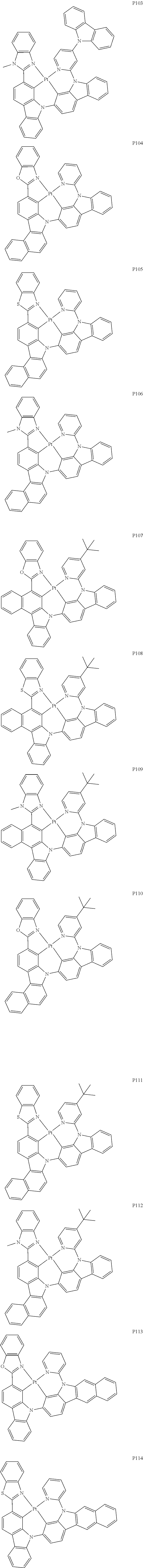

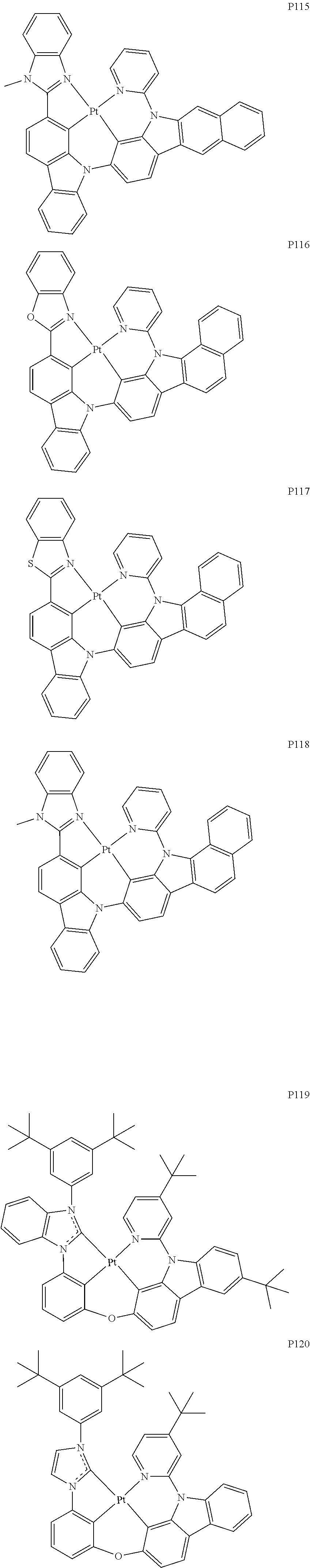

The amount of the phosphorescent complex relative to the total weight of the material for an organic electroluminescent device (in particular, the material for an emission layer) is not particularly limited, but may be 0.1 wt % or more, for example, 0.2 wt % or more. In addition, the amount of the phosphorescent complex may be 0.5 wt % or more, for example, 1 wt % or more. In addition, the amount of the phosphorescent complex may be 3 wt % or more, for example, 5 wt % or more. Within this range, an organic electroluminescent device having excellent luminescence color purity and high luminescence efficiency may be obtained. In addition, the amount of the phosphorescent complex relative to the total weight of the material for an organic electroluminescent device may be 50 wt % or less. In addition, the amount of the phosphorescent complex may be 40 wt % or less, for example, 30 wt % or less. Within this range, an organic electroluminescent device having excellent luminescence color purity and high luminescence efficiency may be obtained. In addition, in an emission layer of an organic electroluminescent device described later, the amount of the phosphorescent complex relative to the total weight of the emission layer may be the same as described above.

When the material for an organic electroluminescent device (in particular, the material for an emission layer) includes the phosphorescent complex, the amount of the phosphorescent complex may be 100 wt % or more based on 100 wt % of the compound of Formula (1). In addition, the amount of the phosphorescent complex may be 150 wt % or more, for example, 200 wt % or more, based on 100 wt % of the compound of Formula (1). Within this range, an organic electroluminescent device having excellent luminescence color purity and high luminescence efficiency may be obtained. In addition, the amount of the phosphorescent complex is not particularly limited, but may be 10,000 wt % or less based on 100 wt % of the compound of Formula (1). In addition, the amount of the phosphorescent complex may be 7,500 wt % or less, for example, 5,000 wt % or less, based on 100 wt % of the compound of Formula (1). Within this range, an organic electroluminescent device having excellent luminescence color purity and high luminescence efficiency may be obtained. In addition, in an emission layer of an organic electroluminescent device described later, the amount (parts by weight) of the phosphorescent complex relative to 100 wt % of the compound of Formula (1) may be the same as described above.

### Host Material

The material for an organic electroluminescent device according to an embodiment may further include a host material in addition to the compound of Formula (1). Excellent luminescence efficiency may be achieved in an organic electroluminescent device by using both the compound of Formula (1) as a dopant material and the host material.

The host material is not particularly limited, and a known host material may be used as the host material. For example, the known host material may be an anthracene derivative, a pyrene derivative, a fluoranthene derivative, a chrysene derivative, a dihydrobenzoanthracene derivative, or a triphenylene derivative. 9-(1-naphthyl)-10-(2-naphthyl)anthracene (Compound HT4) may be used as the anthracene derivative.

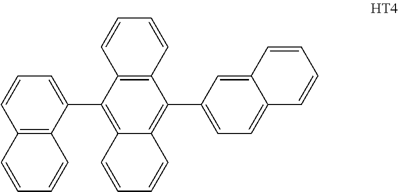 [SMILES: c1ccc2cc(-c3c4ccccc4c(-c4cccc5ccccc45)c4ccccc34)ccc2c1]

Examples of the host material may include a compound having a carbazole ring structure (however, excluding the compound represented by Formula (1)), a compound having a ring structure in which at least one ring-forming carbon atom of a carbazole ring is substituted with a nitrogen atom (however, excluding the compound represented by Formula (1) and the compound having a carbazole ring structure), or a compound having a triazine ring structure (however, excluding the compound represented by Formula (1), the compound having a carbazole ring structure, and the compound having a ring structure in which at least one ring-forming carbon atom of a carbazole ring is substituted with a nitrogen atom). In particular, the host material may be a compound having a carbazole ring structure. By using such compounds as host materials, efficient energy transfer in an emission layer may be promoted. In addition, the balance of carrier mobility between electrons and holes may be improved. In addition, a hydrogen atom that bonds with a ring-forming atom of rings in the carbazole ring structure, the ring structure in which at least one ring-forming carbon atom of a carbazole ring is substituted with a nitrogen atom, and the triazine ring structure of the above compounds may be substituted with other atoms or substituents. In addition, two or more of such substituents may constitute a ring structure.

The compound having a carbazole ring structure or the compound having a ring structure in which at least one ring-forming carbon atom of a carbazole ring is substituted with a nitrogen atom is not particularly limited, but may be a compound having a structure represented by Formula (5).

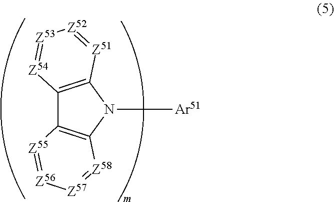

In Formula (5),
Z⁵¹ may be CH, CR⁵¹, or N,
Z⁵² may be CH, CR⁵², or N,
Z⁵³ may be CH, CR⁵³, or N,
Z⁵⁴ may be CH, CR⁵⁴, or N,
Z⁵⁵ may be CH, CR⁵⁵, or N,
Z⁵⁶ may be CH, CR⁵⁶, or N,
Z⁵⁷ may be CH, CR⁵⁷, or N,
Z⁵⁸ may be CH, CR⁵⁸, or N,
R⁵¹ to R⁵⁸ may each independently be a group of any one of (5a) to (5h):
(5a) a cyano group;
(5b) a substituted or unsubstituted alkyl group having 1 to 20 carbon atoms;
(5c) a substituted or unsubstituted alkoxy group having 1 to 20 carbon atoms;
(5d) a substituted or unsubstituted arylamino group having 6 to 20 carbon atoms;
(5e) a substituted or unsubstituted phosphoryl group (a —POH₂ group);
(5f) a substituted or unsubstituted silyl group (a —SiH₃ group);
(5g) a substituted or unsubstituted monovalent aromatic hydrocarbon group; and
(5h) a substituted or unsubstituted monovalent heterocyclic group,
Ar⁵¹ is a group including at least one of an aromatic hydrocarbon group and a heterocyclic group, and
m may be 1, 2, 3, 4, 5, or 6,
wherein R⁵¹ and R⁵², R⁵² and R⁵³, R⁵³ and R⁵⁴, R⁵⁵ and R⁵⁶, R⁵⁶ and R⁵⁷, or R⁵⁷ and R⁵⁸ may optionally form an aliphatic hydrocarbon ring, an aromatic hydrocarbon ring, or a hetero ring, each including bonded carbon atoms.

The descriptions of the groups of (5b) to (5d), (5g), and (5h) in Formula (5) are respectively the same as the descriptions of the groups of (a4) to (a6), (a8), and (a9) in Formula (1).

In addition, the aromatic hydrocarbon group in Ar⁵¹ may be identical to the monovalent aromatic hydrocarbon group provided in the description of the group of (a8) in Formula (1), except that the valency thereof may be different.

In addition, the heterocyclic group in Ar⁵¹ may be identical to the monovalent heterocyclic group provided in the description of the group of (a9) in Formula (1), except that the valency thereof may be different.

In Formula (5), all of Z⁵¹ to Z⁵⁸ may not be N, or one of them may be N. In an embodiment, all of Z⁵¹ to Z⁵⁸ may not be N.

When the groups of (5b) to (5h) in Formula (5) are substituted groups, substituents that are substituted into the groups of (5b) to (5h) are not particularly limited. For example, the substituents may be the groups of (5a) to (5h). Examples of the substituents that are substituted into the groups of (5b) to (5h) are not particularly limited, but may include a cyano group, an unsubstituted alkyl group having 1 to 20 carbon atoms, an alkoxy group having 1 to 20 carbon atoms substituted with a monovalent aromatic hydrocarbon group having 6 to 30 carbon atoms further substituted with an unsubstituted alkenyl group having 2 to 30 carbon atoms, a substituted or unsubstituted arylamino group having 6 to 20 carbon atoms, an unsubstituted monovalent aromatic hydrocarbon group having 6 to 30 carbon atoms, a monovalent aromatic hydrocarbon group having 6 to 30 carbon atoms substituted with a cyano group, a monovalent aromatic hydrocarbon group having 6 to 30 carbon atoms substituted with an unsubstituted alkenyl group having 2 to 30 carbon atoms, a monovalent aromatic hydrocarbon group having 6 to 30 carbon atoms substituted with an unsubstituted arylamino group having 6 to 20 carbon atoms, an unsubstituted monovalent heterocyclic group having 3 to 30 ring-forming atoms, a monovalent heterocyclic group having 3 to 30 ring-forming atoms substituted with an unsubstituted monovalent aromatic hydrocarbon group having 6 to 30 carbon atoms.

In Formula (5), Ar⁵¹ is not particularly limited as long as Ar⁵¹ is a group including at least one of an aromatic hydrocarbon group and a heterocyclic group. For example, examples of Ar⁵¹ may include a substituted or unsubstituted aromatic hydrocarbon group, a substituted or unsubstituted heterocyclic group, a group in which at least one substituted or unsubstituted aromatic hydrocarbon group is bonded to at least one substituted or unsubstituted heterocyclic group via a single bond, or a group in which at least two substituted or unsubstituted aromatic hydrocarbon groups or substituted or unsubstituted heterocyclic groups are bonded to each other via a linking group other than the at least two groups.

In this regard, in the group in which at least two substituted or unsubstituted aromatic hydrocarbon groups or substituted or unsubstituted heterocyclic groups are bonded to each other via a linking group other than the at least two groups, the linking group is not particularly limited. Examples of the linking group may include a Si group, a N group, a P═O group, a S(═O)═O group, a C═O group, or the like.

When groups constituting Ar⁵¹ in Formula (5) are substituted groups, substituents that are substituted into such groups are not particularly limited. For example, the substituents may be the groups of (5a) to (5h). Examples of the substituents that are substituted into such groups are not particularly limited, but may include a cyano group, an unsubstituted alkyl group having 1 to 20 carbon atoms, a monovalent heterocyclic group having 3 to 30 ring-forming atoms substituted with an unsubstituted alkyl group having 1 to 20 carbon atoms, or the like.

In this regard, in the substituents of the groups of (5b) to (5h) or the substituents of the groups constituting Ar⁵¹, the alkyl group having 1 to 20 carbon atoms, the alkoxy group having 1 to 20 carbon atoms, the arylamino group having 6 to 20 carbon atoms, the monovalent aromatic hydrocarbon group having 6 to 30 carbon atoms, and the monovalent heterocyclic group having 3 to 30 ring-forming atoms are respectively the same as the descriptions of the groups of (a4) to (a10) in Formula (1).

In addition, the alkenyl group having 2 to 30 carbon atoms in the substituents of the groups of (5c) to (5h) or the substituents of the groups constituting Ar⁵¹ is not particularly limited, and may be linear, branched, or cyclic. Examples of the alkenyl group are not particularly limited, but may include, for example, a vinyl group, a 2-prophenyl group, a 2-butenyl group, a 3-butenyl group, a 1-methyl-2-prophenyl group, a 2-methyl-2-prophenyl group, a 2-pentenyl group, a 3-pententyl group, a 4-pentenyl group, a 1-methyl-2-butenyl group, a 2-methyl-2-butenyl group, a 3-methyl-2-butenyl group, a 1-methyl-3-butenyl group, a 2-methyl-3-butenyl group, a 3-methyl-3-butenyl group, a 1,1-dimethyl-2-prophenyl group, a 1,2-dimethyl-2-prophenyl group, a 1-ethyl-2-prophenyl group, or the like.

In Formula (5), m may be 1, 2, 3, or 4, for example, 2.

Hereinafter, as the host material according to an embodiment, the compound having a carbazole ring structure and the compound having a ring structure in which at least one ring-forming carbon atom of a carbazole ring is substituted with a nitrogen atom are described in detail. However, the disclosure is not limited to such examples.

The host material may include one of compounds H1 to H88, HT1 to HT2, or a combination thereof.

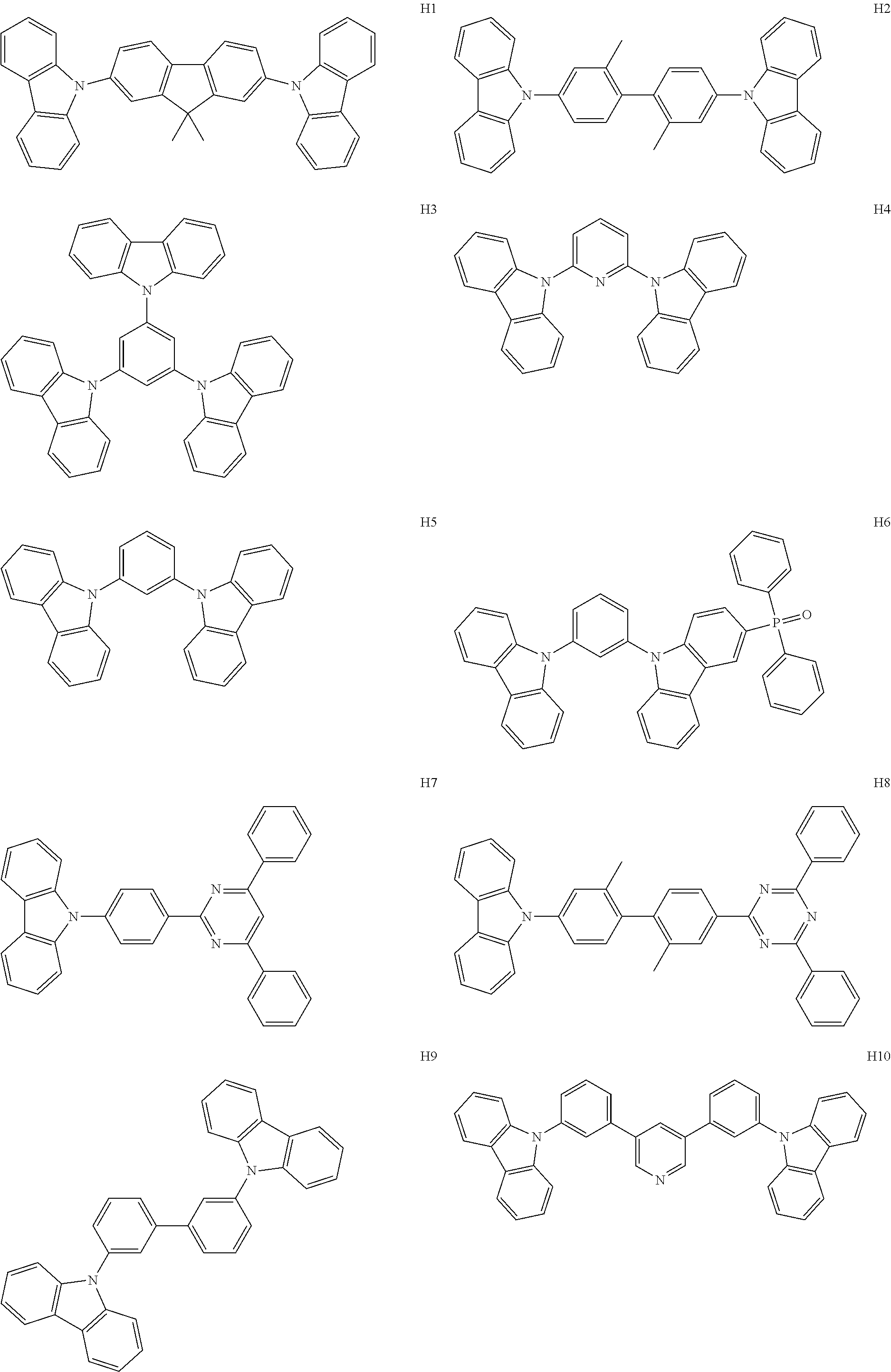

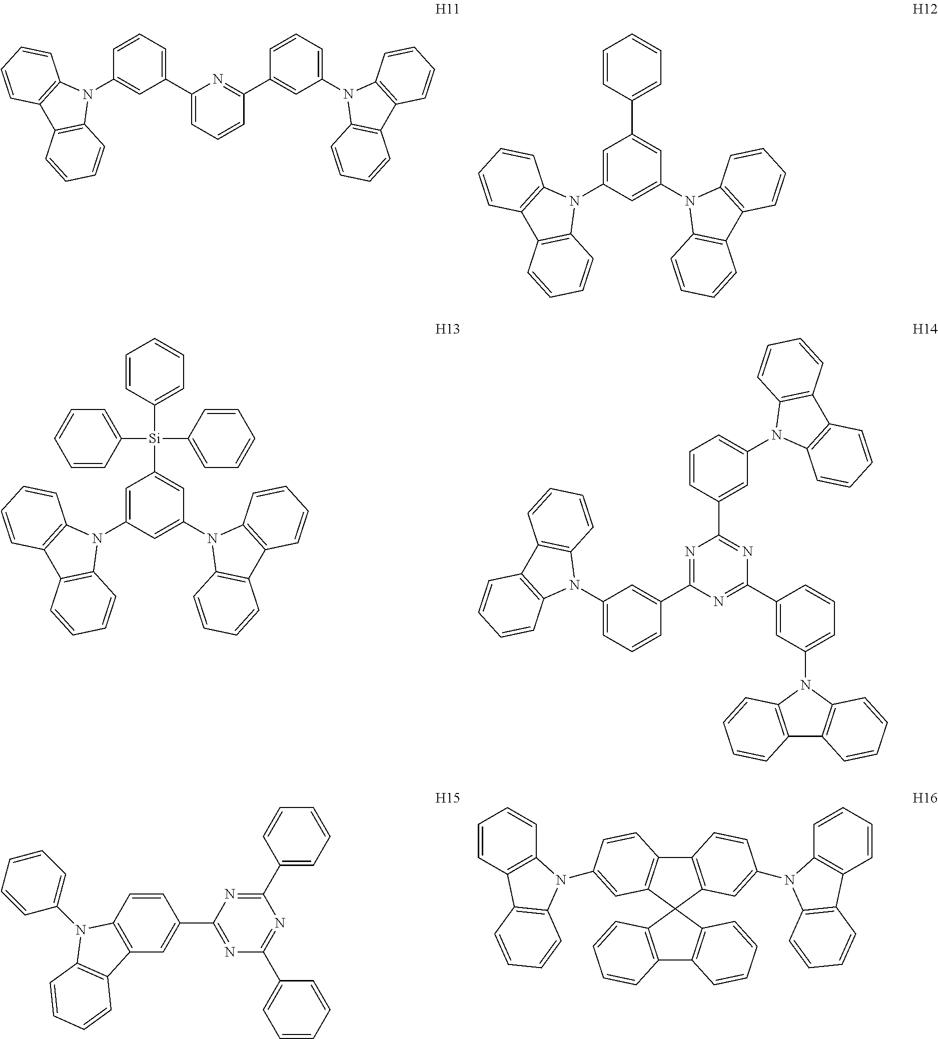

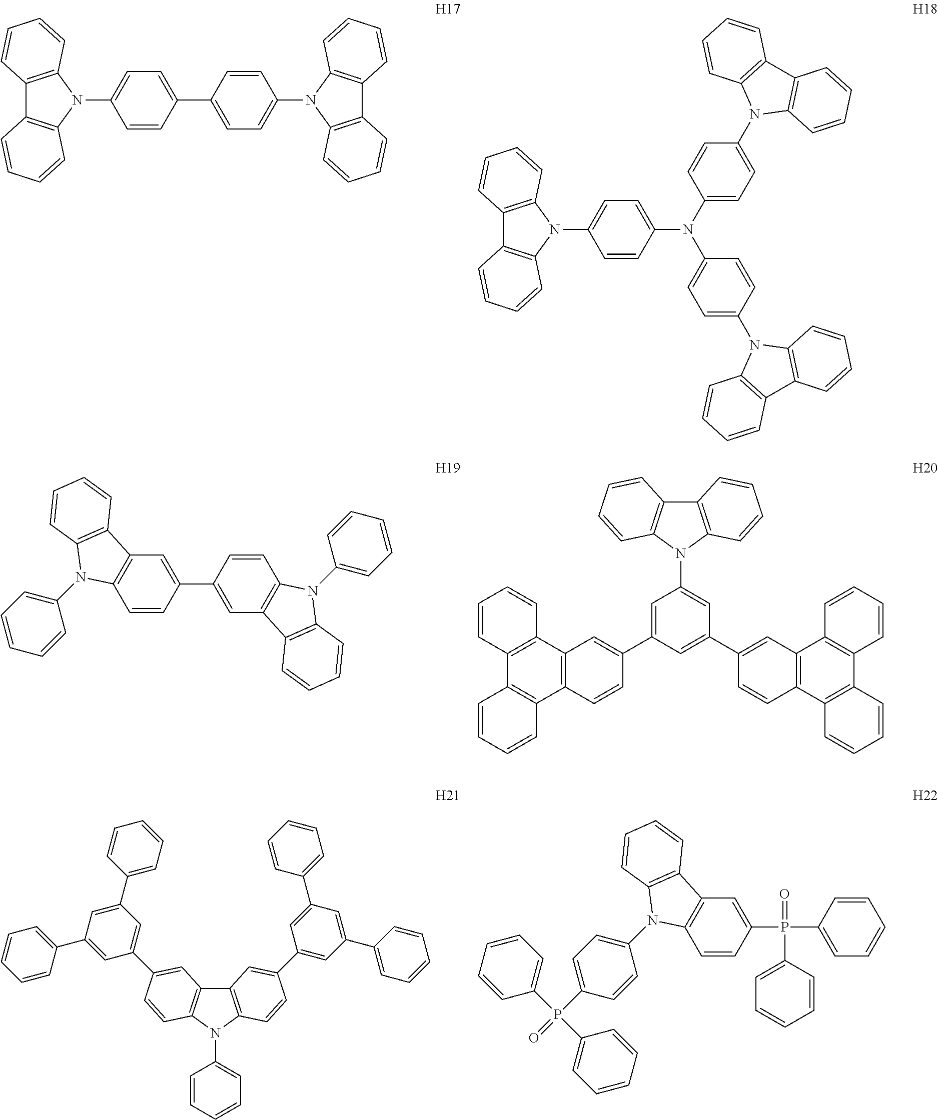

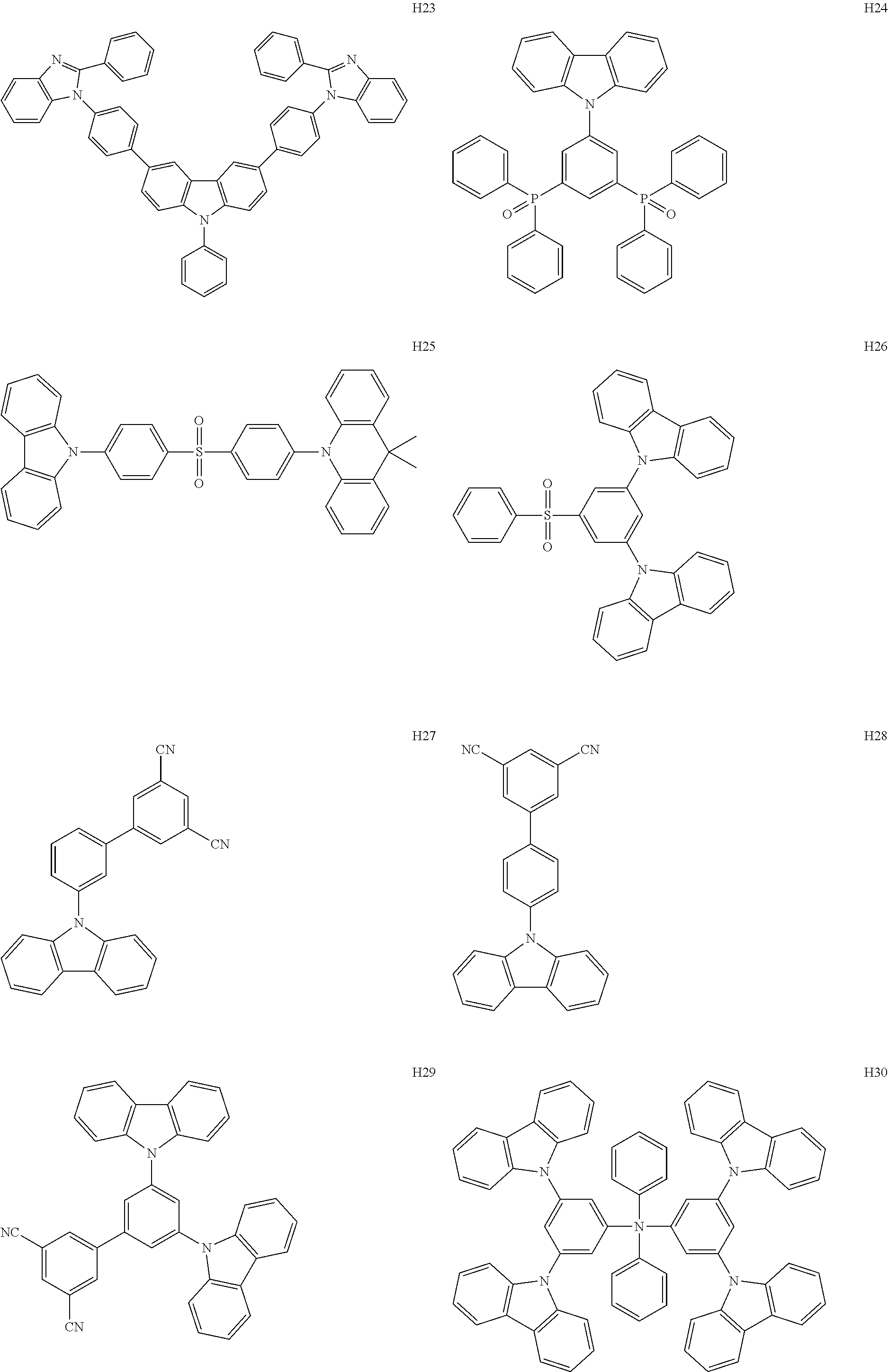

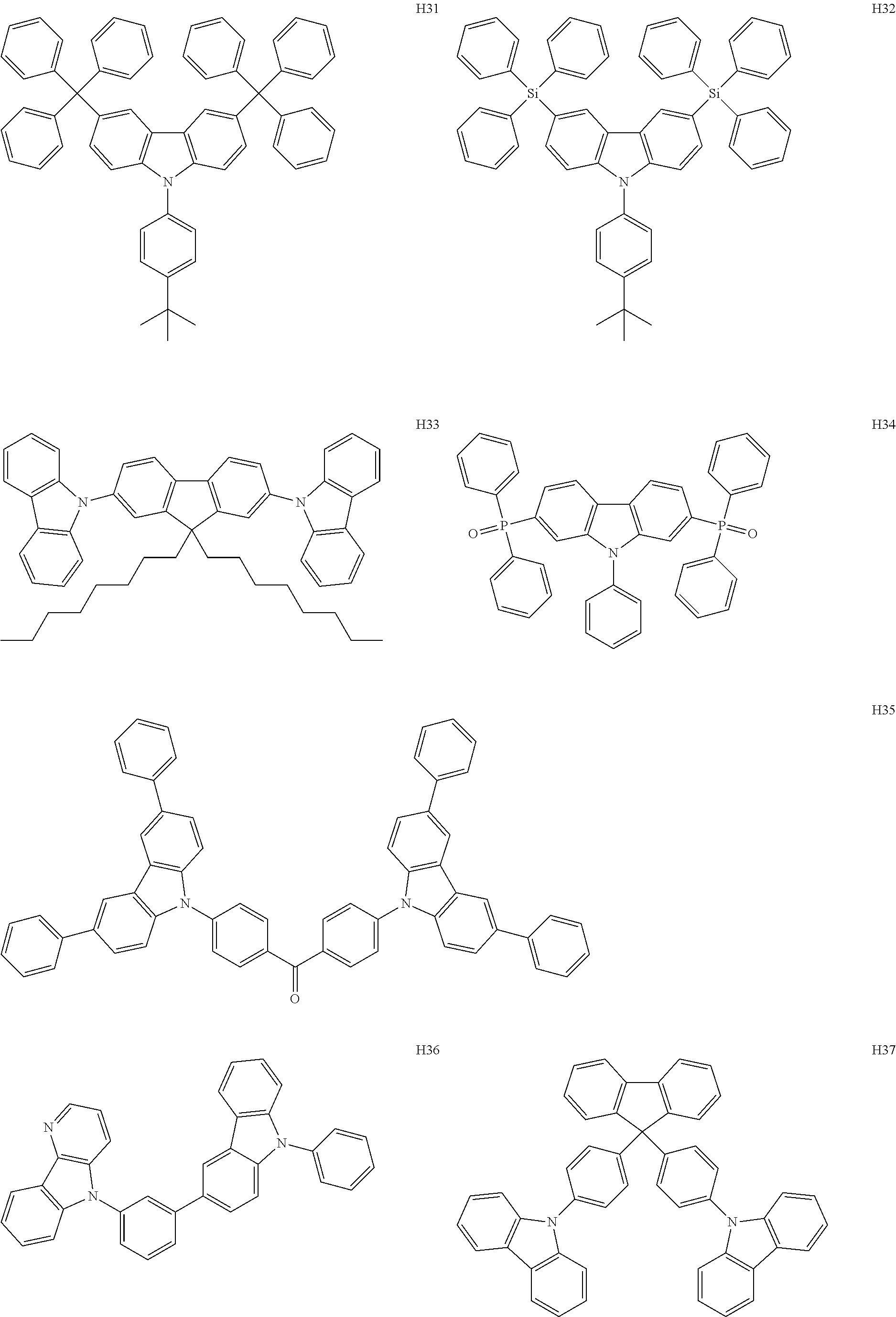

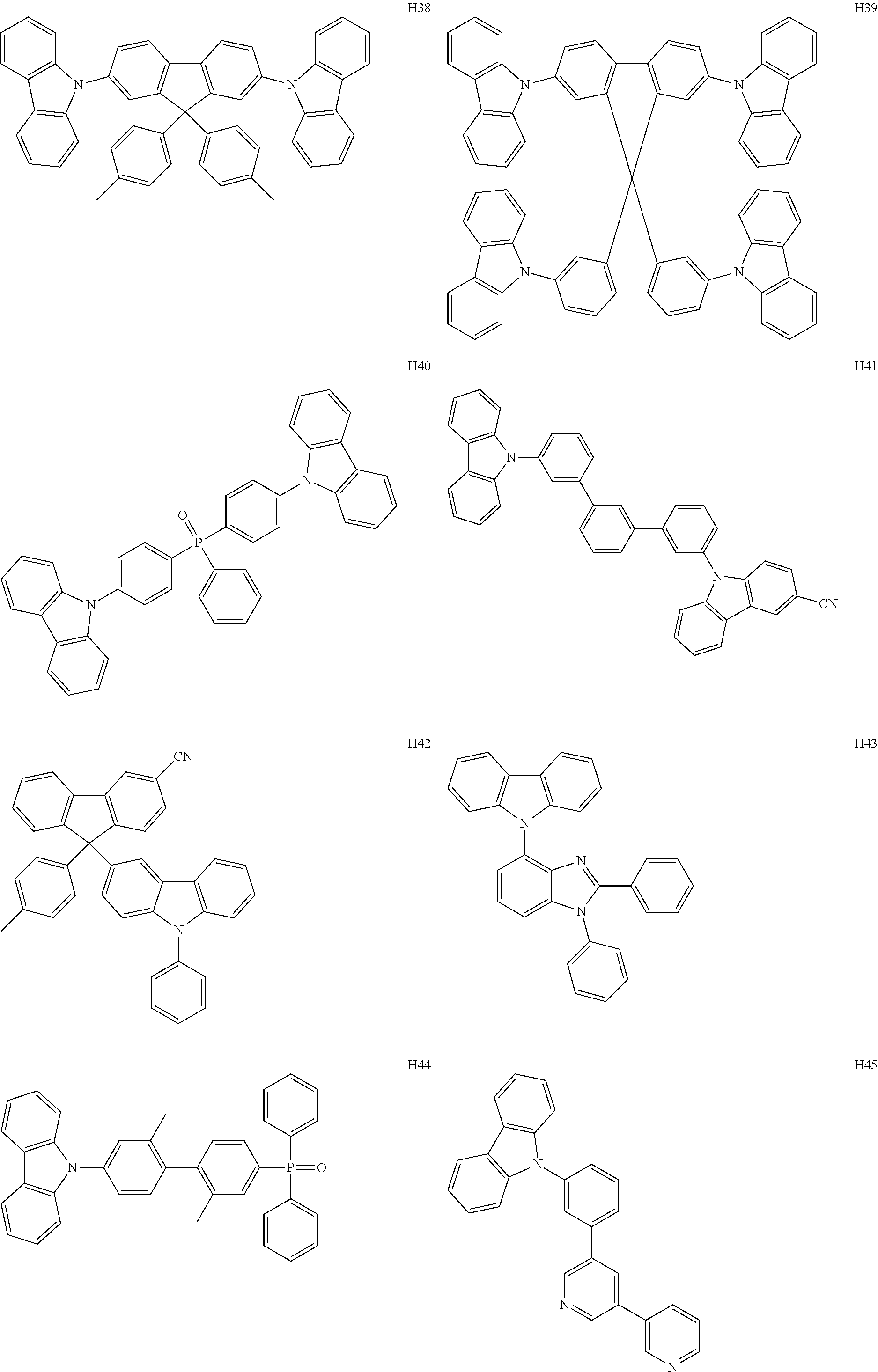

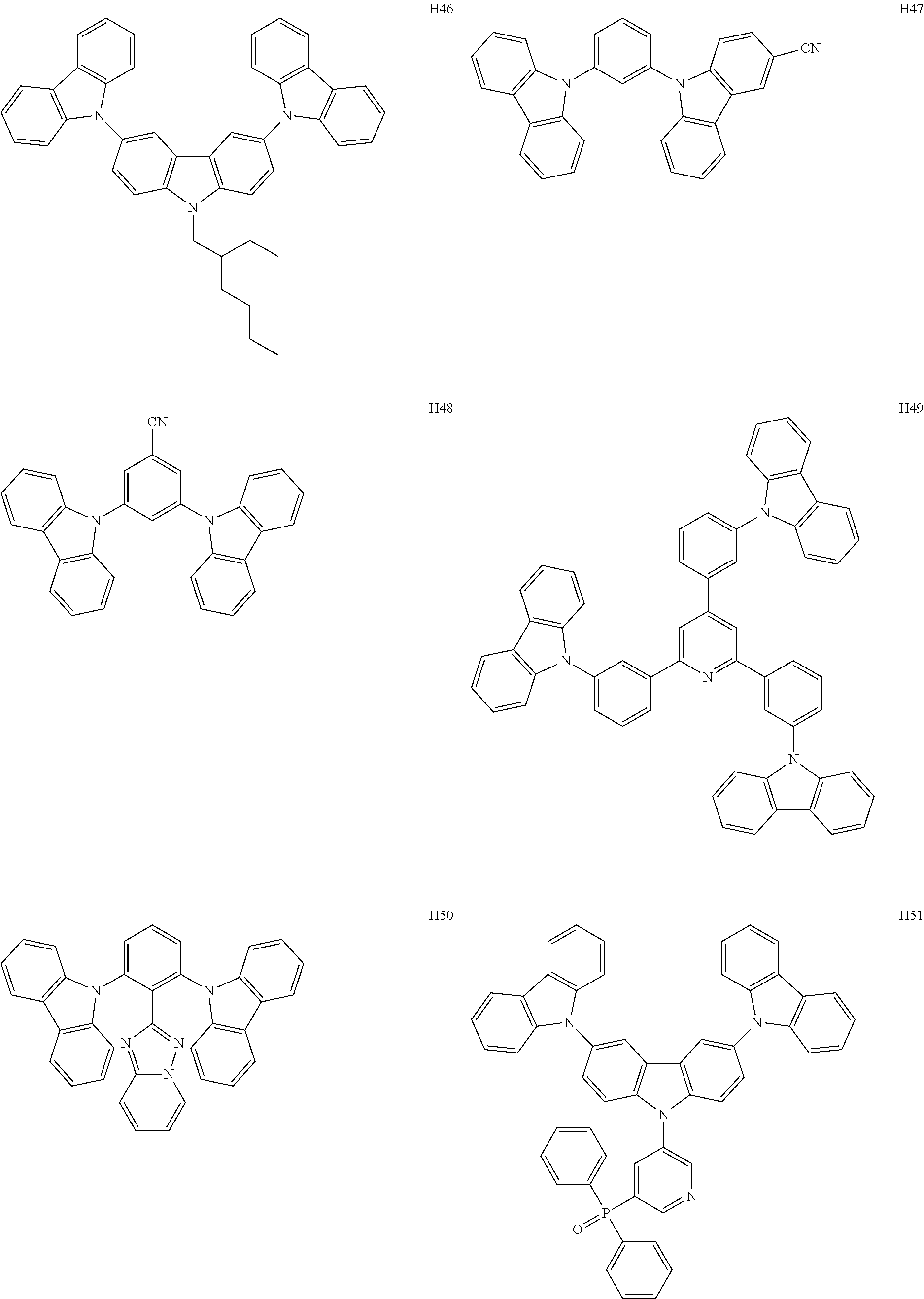 [SMILES: CCCCC(CC)Cn1c2ccc(-n3c4ccccc4c4ccccc43)cc2c2cc(-n3c4ccccc4c4ccccc43)ccc21 ; N#Cc1ccc2c(c1)c1ccccc1n2-c1cccc(-n2c3ccccc3c3ccccc32)c1 ; N#Cc1cc(-n2c3ccccc3c3ccccc32)cc(-n2c3ccccc3c3ccccc32)c1 ; c1cc(-c2cc(-c3cccc(-n4c5ccccc5c5ccccc54)c3)nc(-c3cccc(-n4c5ccccc5c5ccccc54)c3)c2)cc(-n2c3ccccc3c3ccccc32)c1 ; c1cc(-n2c3ccccc3c3ccccc32)c(-c2nc3ccccn3n2)c(-n2c3ccccc3c3ccccc32)c1 ; O=P(c1ccccc1)(c1ccccc1)c1cncc(-n2c3ccc(-n4c5ccccc5c5ccccc54)cc3c3cc(-n4c5ccccc5c5ccccc54)ccc32)c1]

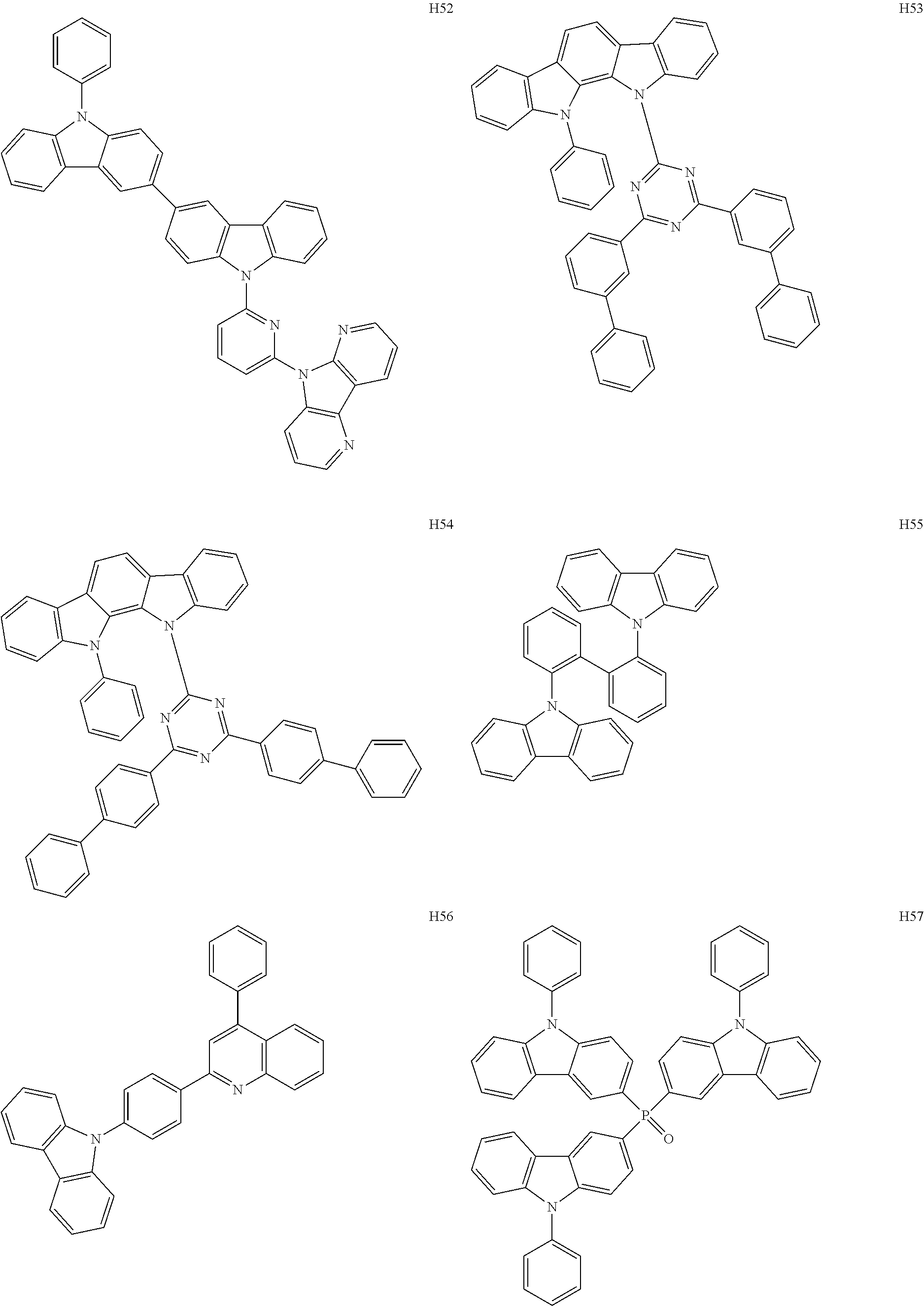

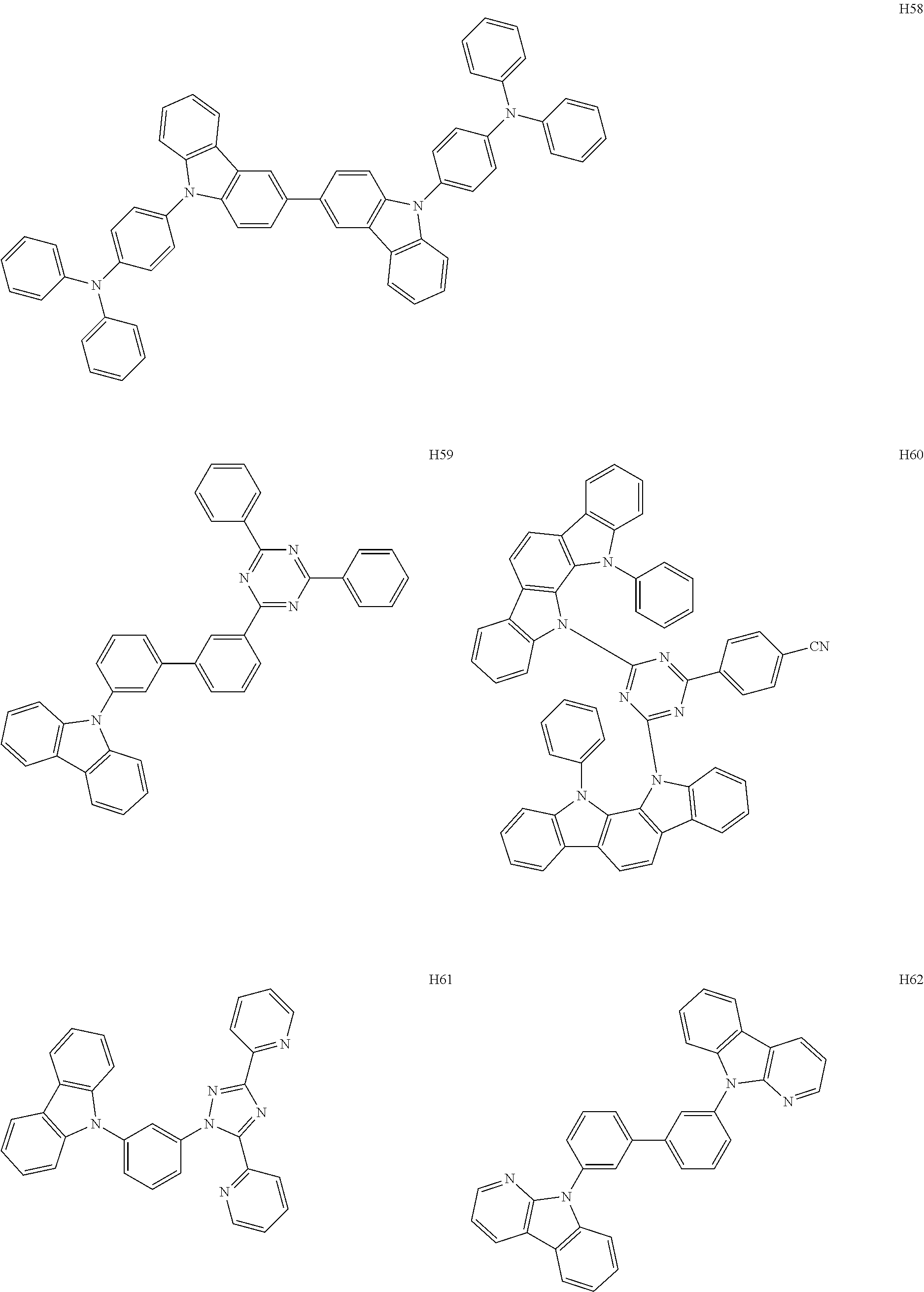 [SMILES: c1ccc(N(c2ccccc2)c2ccc(-n3c4ccccc4c4cc(-c5ccc6c(c5)c5ccccc5n6-c5ccc(N(c6ccccc6)c6ccccc6)cc5)ccc43)cc2)cc1 ; c1ccc(-c2nc(-c3ccccc3)nc(-c3cccc(-c4cccc(-n5c6ccccc6c6ccccc65)c4)c3)n2)cc1 ; N#Cc1ccc(-c2nc(-n3c4ccccc4c4ccc5c6ccccc6n(-c6ccccc6)c5c43)nc(-n3c4ccccc4c4ccc5c6ccccc6n(-c6ccccc6)c5c43)n2)cc1 ; c1ccc(-c2nc(-c3ccccn3)n(-c3cccc(-n4c5ccccc5c5ccccc54)c3)n2)nc1 ; c1cc(-c2cccc(-n3c4ccccc4c4cccnc43)c2)cc(-n2c3ccccc3c3cccnc32)c1]

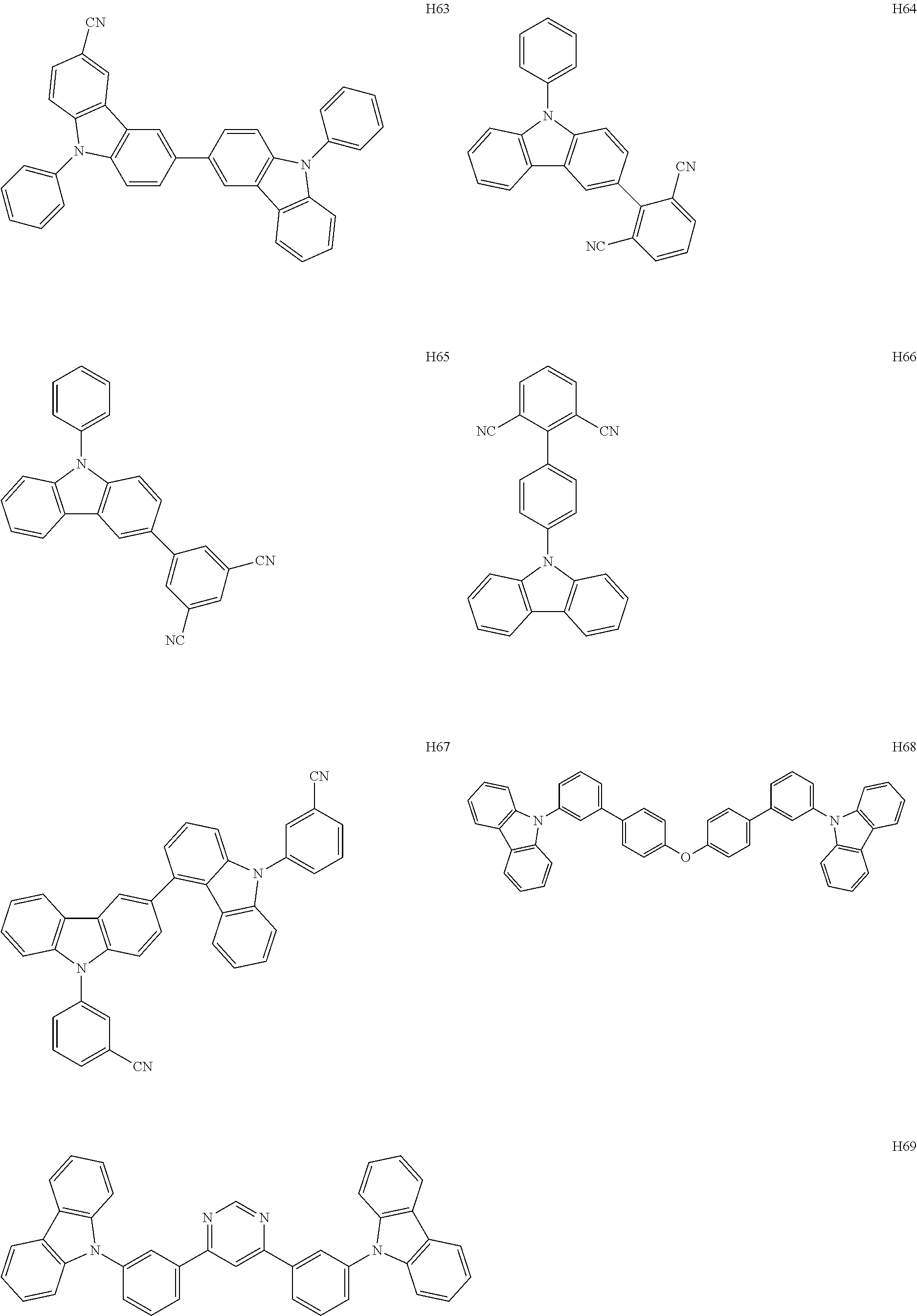

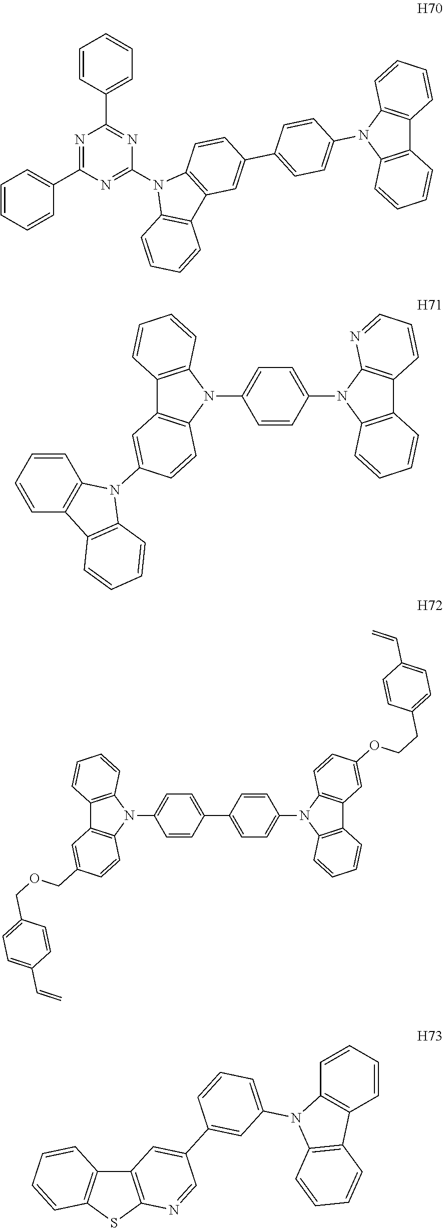 [SMILES: c1ccc(-c2nc(-c3ccccc3)nc(-n3c4ccccc4c4cc(-c5ccc(-n6c7ccccc7c7ccccc76)cc5)ccc43)n2)cc1 ; c1ccc2c(c1)c1ccccc1n2-c1ccc2c(c1)c1ccccc1n2-c1ccc(-n2c3ccccc3c3cccnc32)cc1 ; C=Cc1ccc(CCOc2ccc3c(c2)c2ccccc2n3-c2ccc(-c3ccc(-n4c5ccccc5c5cc(COCc6ccc(C=C)cc6)ccc54)cc3)cc2)cc1 ; c1cc(-c2cnc3sc4ccccc4c3c2)cc(-n2c3ccccc3c3ccccc32)c1]

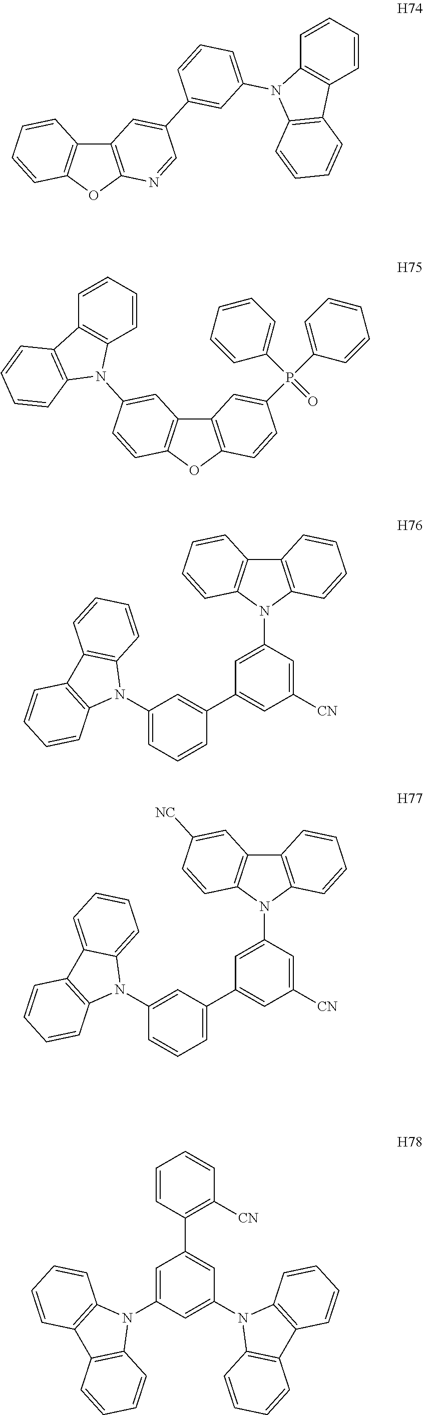 [SMILES: c1cc(-c2cnc3oc4ccccc4c3c2)cc(-n2c3ccccc3c3ccccc32)c1 ; O=P(c1ccccc1)(c1ccccc1)c1ccc2oc3ccc(-n4c5ccccc5c5ccccc54)cc3c2c1 ; N#Cc1cc(-c2cccc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1ccccc1-c1cc(-n2c3ccccc3c3ccccc32)cc(-n2c3ccccc3c3ccccc32)c1]

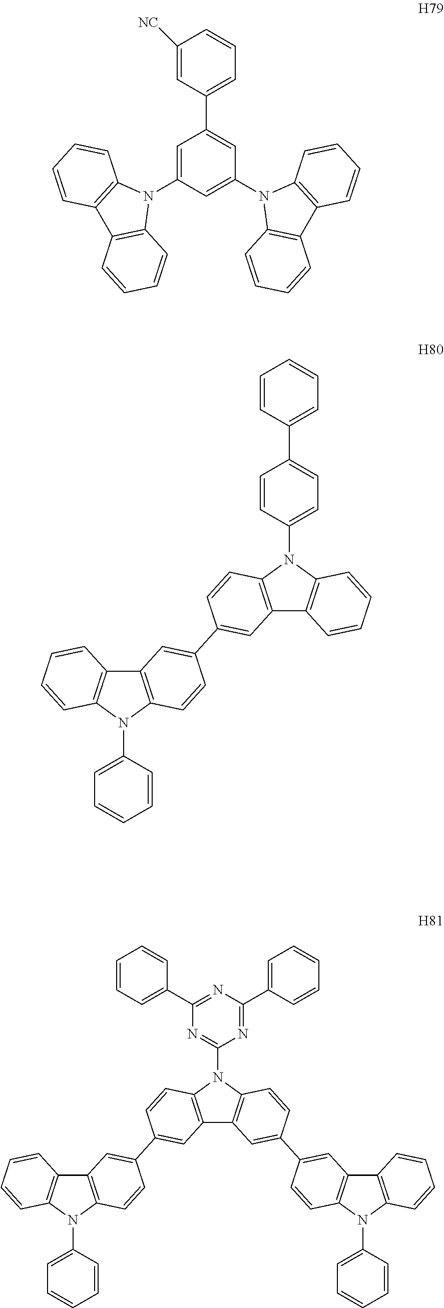 [SMILES: N#Cc1cccc(-c2cc(-n3c4ccccc4c4ccccc43)cc(-n3c4ccccc4c4ccccc43)c2)c1 ; c1ccc(-c2ccc(-n3c4ccccc4c4cc(-c5ccc6c(c5)c5ccccc5n6-c5ccccc5)ccc43)cc2)cc1 ; c1ccc(-c2nc(-c3ccccc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

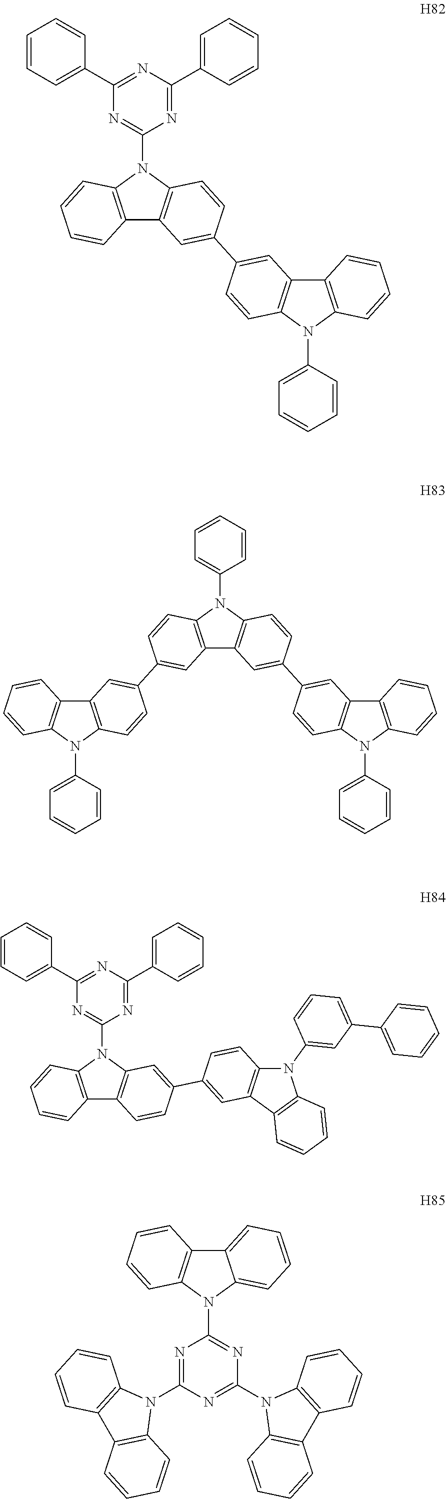 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2cccc(-n3c4ccccc4c4cc(-c5ccc6c7ccccc7n(-c7nc(-c8ccccc8)nc(-c8ccccc8)n7)c6c5)ccc43)c2)cc1 ; c1ccc2c(c1)c1ccccc1n2-c1nc(-n2c3ccccc3c3ccccc32)nc(-n2c3ccccc3c3ccccc32)n1]

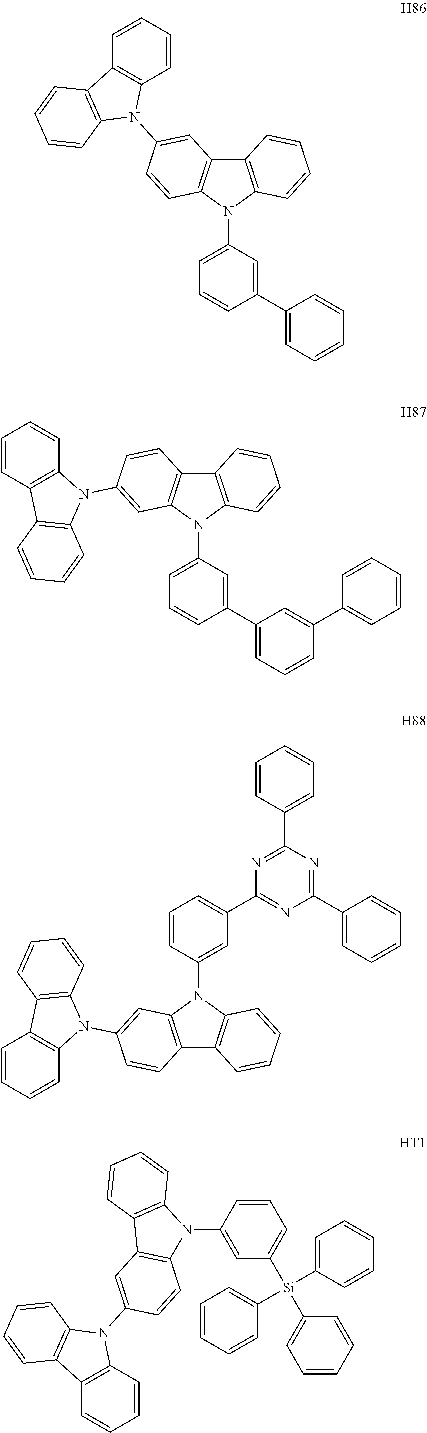 [SMILES: c1ccc(-c2cccc(-n3c4ccccc4c4cc(-n5c6ccccc6c6ccccc65)ccc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2nc(-c3ccccc3)nc(-c3cccc(-n4c5ccccc5c5ccc(-n6c7ccccc7c7ccccc76)cc54)c3)n2)cc1 ; c1ccc([Si](c2ccccc2)(c2ccccc2)c2cccc(-n3c4ccccc4c4cc(-n5c6ccccc6c6ccccc65)ccc43)c2)cc1]

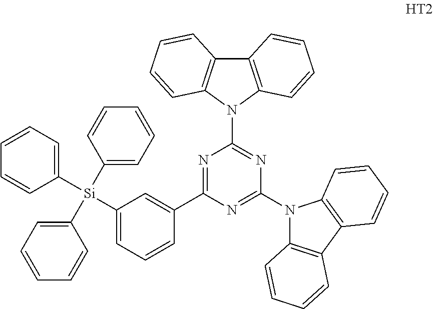 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1]

Therefore, the material for an organic EL device according to an embodiment may include the compound of Formula (1), the phosphorescent complex, and the host material, wherein the host material may include the compound having the structure represented by Formula (5). In addition, an organic electroluminescent device according to an embodiment, which is described later, may include the compound of Formula (1) and the host material, wherein the host material may include the compound having the structure represented by Formula (5). In addition, an organic electroluminescent device according to an embodiment, which is described later, may include the compound of Formula (1), the phosphorescent complex, and the host material, wherein the host material may include at least two compounds having the structure represented by Formula (5). For example, the compound having the structure represented by Formula (5) may include HT1 and HT2.

The compound having a triazine ring structure is not particularly limited, but may be a compound having a structure represented by Formula (6).

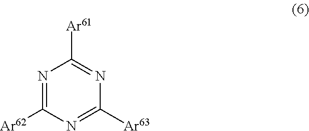

In Formula (6),
Ar⁶¹ to Ar⁶³ may each independently be a substituted or unsubstituted monovalent aromatic hydrocarbon group or a substituted or unsubstituted monovalent heterocyclic group.

In Formula (6), the substituted or unsubstituted monovalent aromatic hydrocarbon group is as described in the description of the group of (a8) in Formula (1). In addition, the substituted or unsubstituted monovalent heterocyclic group is as described in the description of the group of (a9) in Formula (1).

A substituent that is substituted into the monovalent aromatic hydrocarbon group or the monovalent heterocyclic group in Formula (6) is not particularly limited, but may be a substituent that is substituted into the groups of (a4) to (a10) in Formula (1). In addition, the substituent may be a silyl group substituted with an unsubstituted monovalent aromatic hydrocarbon group. In addition, the unsubstituted monovalent aromatic hydrocarbon group is as described in the description of the unsubstituted group of (a8).

For the compound having a triazine ring structure, a compound including a silyl group (a compound having a triazine ring structure with a silyl group) may be preferred.

In addition, the compound having a triazine ring structure may be used in combination with the compound having a carbazole ring structure or the compound having a ring structure in which at least one ring-forming carbon atom of a carbazole ring is substituted with a nitrogen atom.

Hereinafter, the compound having a triazine structure, which is the host material according to an embodiment, is described in detail. However, the disclosure is not limited to such examples. Examples of the compound having a triazine structure include H86, H87, H88, HT2, or the like.

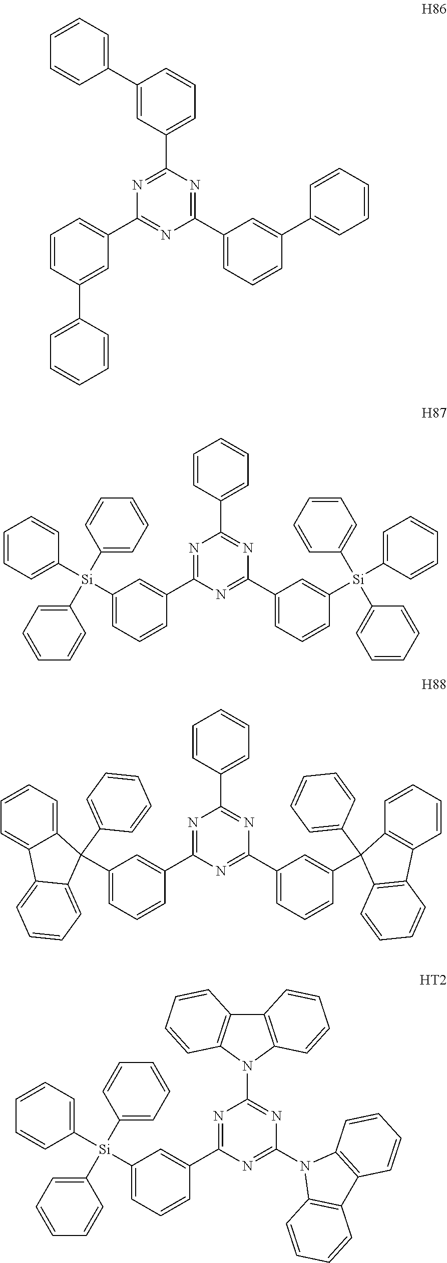 [SMILES: c1ccc(-c2cccc(-c3nc(-c4cccc(-c5ccccc5)c4)nc(-c4cccc(-c5ccccc5)c4)n3)c2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2nc(-c3cccc(C4(c5ccccc5)c5ccccc5-c5ccccc54)c3)nc(-c3cccc(C4(c5ccccc5)c5ccccc5-c5ccccc54)c3)n2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

Therefore, the material for an organic electroluminescent device according to an embodiment may include the compound of Formula (1), the phosphorescent complex, and the host material, wherein the host material may include the compound having the structure represented by Formula (6). In addition, the material for an organic electroluminescent device according to an embodiment may include the compound of Formula (1), the phosphorescent complex, and the host material, wherein the host material may include the compound having the structure represented by Formula (5) and the compound having the structure represented by Formula (6). In addition, an organic electroluminescent device according to an embodiment, which is described later, may include the compound of Formula (1) and the host material, wherein the host material may include the compound having the structure represented by Formula (6). In addition, an organic electroluminescent device according to an embodiment may include the compound of Formula (1) and the host material, wherein the host material may include the compound having the structure represented by Formula (5) and the compound having the structure represented by Formula (6).

The amount of the host material relative to the total weight of the material for an organic electroluminescent device (in particular, the material for an emission layer) is not particularly limited, but may be 5 wt % or more. In addition, the amount of the host material may be 10 wt % or more, for example, 20 wt % or more. Within this range, an organic electroluminescent device having excellent luminescence color purity and high luminescence efficiency may be obtained. In addition, the amount of the host material relative to the total weight of the material for an organic electroluminescent device (in particular, the material for an emission layer) is not particularly limited, but may be 99 wt % or less. In addition, the amount of the host material may be 98 wt % or less, for example, 95 wt % or less. Within this range, an organic electroluminescent device having excellent luminescence color purity and high luminescence efficiency may be obtained. In addition, in an emission layer of an organic electroluminescent device described later, the amount of the host material relative to the total weight of the emission layer may be the same as described above.

When the material for an organic electroluminescent device (in particular, the material for an emission layer) includes the host material, the amount of the host material may be 1,000 wt % or more based on 100 wt % of the compound of Formula (1). In addition, the amount of the host material may be 2,000 wt % or more, for example, 3,000 wt % or more, based on 100 wt % of the compound of Formula (1). Within this range, an organic electroluminescent device having excellent luminescence color purity and high luminescence efficiency may be obtained. In addition, the amount of the host material is not particularly limited, but may be 200,000 wt % or less based on 100 wt % of the compound of Formula (1). In addition, the amount of the host material may be 150,000 wt % or less, for example, 100,000 wt % or less, based on 100 wt % of the compound of Formula (1). Within this range, an organic electroluminescent device having excellent luminescence color purity and high luminescence efficiency may be obtained. In addition, in an emission layer of an organic electroluminescent device described later, the amount (parts by weight) of the host material relative to 100 wt % of the compound of Formula (1) may be the same as described above.

### Liquid Composition

Another aspect of the disclosure relates to a liquid composition including the compound of Formula (1), the material for an organic electroluminescent device, and a solvent.

The solvent is not particularly limited, and may be a solvent having a boiling point of at least about 100° C. and not more than about 350° C. at atmospheric pressure (101.3 kilopascals (kPa), 1 atmosphere (atm)). The boiling point of the solvent at atmospheric pressure may be about 150° C. to about 320° C., for example, about 180° C. to about 300° C. When the boiling point of the solvent at atmospheric pressure is within the ranges above, the processability or film-forming capability of a wet film forming method, in particular, an inkjet method, may be improved. The solvent having a boiling point of about 100° C. to about 350° C. at atmospheric pressure is not particularly limited, and a known solvent may be appropriately used. Hereinafter, the solvent having a boiling point of about 100° C. to about 350° C. at atmospheric pressure is described in detail, but the disclosure is not limited thereto. Examples of a hydrocarbon-based solvent may include octane, nonane, decane, undecane, dodecane, or the like. Examples of an aromatic hydrocarbon-based solvent may include toluene, xylene, ethylbenzene, n-propylbenzene, iso-propylbenzene, mesitylene, n-butylbenzene, sec-butylbenzene, 1-phenylpentane, 2-phenylpentane, 3-phenylpentane, phenylcyclopentane, phenylcyclohexane, 2-ethylbiphenyl, 3-ethylbiphenyl, or the like. Examples of an ether-based solvent may include 1,4-dioxane, 1,2-diethoxyethane, diethyleneglycoldimethylether, diethyleneglycoldiethylether, anisole, ethoxybenzene, 3-methylanisole, m-dimethoxybenzene, etc. Examples of a ketone-based solvent may include 2-hexanone, 3-hexanone, cyclohexanone, 2-heptanone, 3-heptanone, 4-heptanone, cycloheptanone, or the like. Examples of an ester-based solvent may include butylacetate, butylpropionate, butylbutyrate, propylenecarbonate, methylbenzoate, ethylbenzoate, 1-propylbenzoate, 1-butylbenzoate, or the like. Examples of a nitrile-based solvent may include benzonitrile, 3-methylbenzonitrile, or the like. Examples of an amide-based solvent may include dimethylformamide, dimethylacetamide, N-methylpyrrolidone, or the like. Such solvents may be used alone or in combination of two or more.

In an embodiment, the amounts of the compound of Formula (1) and the material for an organic electroluminescent device in the liquid composition are not particularly limited.

In an embodiment, the liquid composition may be used as a coating liquid for forming an organic layer of an organic electroluminescent device. In addition, the liquid composition may be used as a coating liquid for forming an emission layer among coating liquids for forming an organic layer.

### Organic Electroluminescent Device

Another aspect of the disclosure relates to an organic electroluminescent device having an emission layer including the compound of Formula (1). In addition, the emission layer in the organic electroluminescent device may include the compound of Formula (1) and a phosphorescent complex, wherein the emission layer may further include a host material in addition to the compound of Formula (1) and the phosphorescent complex. At this time, the phosphorescent complex may be a platinum complex.

In addition, another aspect of the disclosure relates to an organic electroluminescent device including the material for an organic electroluminescent device. In addition, the material for an organic electroluminescent device in the organic electroluminescent device may further include the host material. In addition, the phosphorescent complex included in the organic electroluminescent device may be a platinum complex.

The host material included in the organic electroluminescent device may be the compound having a carbazole ring structure, the compound having a ring structure in which at least one ring-forming carbon atom of a carbazole ring is substituted with a nitrogen atom, or the compound having a triazine ring structure. In addition, the host material included in the organic electroluminescent device may include the compound having the structure represented by Formula (5). In addition, the host material included in the organic electroluminescent device may include the compound having the structure represented by Formula (6). In addition, the host material included in the organic electroluminescent device may include the compound having the structure represented by Formula (5) and the compound having the structure represented by Formula (6).

In addition, the phosphorescent complex included in the organic electroluminescent device may be the compound having the structure represented by Formula (4).

The organic electroluminescent device according to an embodiment is not particularly limited, and may include, for example, a first electrode, a second electrode, and a single organic layer or a plurality of organic layers. The second electrode may be disposed on the first electrode.

Herein, when a portion of a layer, film, region, plate, or the like is said to be “on” or “above” another portion, this includes not only a case where the portion is “directly on” the other portion, but also a case where an intervening layer is present therebetween. In contrast, when a portion of a layer, film, region, plate, or the like is said to be “under” or “below” another portion, this includes not only a case where the portion is “directly under” the other portion, but also a case where an intervening layer is present therebetween. Herein, being disposed “on” includes not only being disposed on the top surface but also on the lower or bottom surface.

The organic electroluminescent device according to an embodiment may include a first electrode, a second electrode, and a single layer or a plurality of layers between the first electrode and the second electrode. In this regard, the layer or layers may include at least one organic layer, and at least one of organic layers may include the compound of Formula (1) or the material for an organic electroluminescent device. An organic layer including the compound of Formula (1) or the material for an organic electroluminescent device may include the emission layer. Luminescence with high color purity may be realized by these organic electroluminescent devices.

As such, the emission layer may include at least one of the compound of Formula (1).

The emission layer may be a single layer consisting of a single material or a single layer consisting of a plurality of different materials. In addition, the emission layer may have a multilayer structure having a plurality of layers consisting of a plurality of different materials.

The emission layer is not particularly limited, and may include, for example, a host material and a dopant material. The compound of Formula (1) may be used as a host material or a dopant material, and for example, may be used as a dopant material.

In an embodiment, the organic electroluminescent device may include an emission layer, wherein the emission layer may include the compound of Formula (1) or the material for an organic electroluminescent device. In addition, the emission layer may include the material for an organic electroluminescent device. In view of a peak wavelength in an emission spectrum, luminescence color purity, and luminescence efficiency, the material for an organic electroluminescent device may include the host material in addition to the compound of Formula (1). In an embodiment, the material for an organic electroluminescent device may include the phosphorescent complex and the host material in addition to the compound of Formula (1). In addition, the amount or proportion of each of the compound of Formula (1), the phosphorescent complex, and the host material in the emission layer may be the same as the amount or proportion of the material for an organic electroluminescent device.

The thickness of the emission layer is not particularly limited, but may be at least about 1 nm and not more than about 100 nm, for example, at least about 10 nm and not more than about 50 nm.

Examples of a film forming method of the emission layer are not particularly limited, but may include known film forming methods such as vacuum deposition, spin coating, Langmuir-Blodgett (LB) deposition, ink-jet printing, laser-printing, or laser-induced thermal imaging (LITI).

An emission wavelength of the organic electroluminescent device is not particularly limited. The emission wavelength of the organic electroluminescent device may be the same as, for example, the peak wavelength of emission of the PL of the compound of Formula (1) according to the disclosure. In particular, in specifications of currently commercialized products, in the case of blue luminescence, the organic electroluminescent device may emit light having a peak in a wavelength region of about 445 nm to about 470 nm, for example, about 450 nm to about 470 nm, for example, about 450 nm to about 465 nm.

In addition, the FWHM of a peak of an emission spectrum of the organic electroluminescent device may be decreased. The FWHM of the peak of the emission spectrum may be 30 nm or less, for example, 25 nm or less. The FWHM of the peak of the emission spectrum may be 20 nm or less (the lower limit is greater than 0 nm).

Hereinafter, a case where the organic electroluminescent device according to an embodiment further includes an organic layer in addition to the emission layer is described in detail with reference to the accompanying drawings. In the description of the drawings, the same components will be denoted by the same reference numerals, and redundant descriptions thereof will be omitted. In addition, dimensional ratios in the drawings may be exaggerated for convenience of description, and thus may differ from actual ratios.

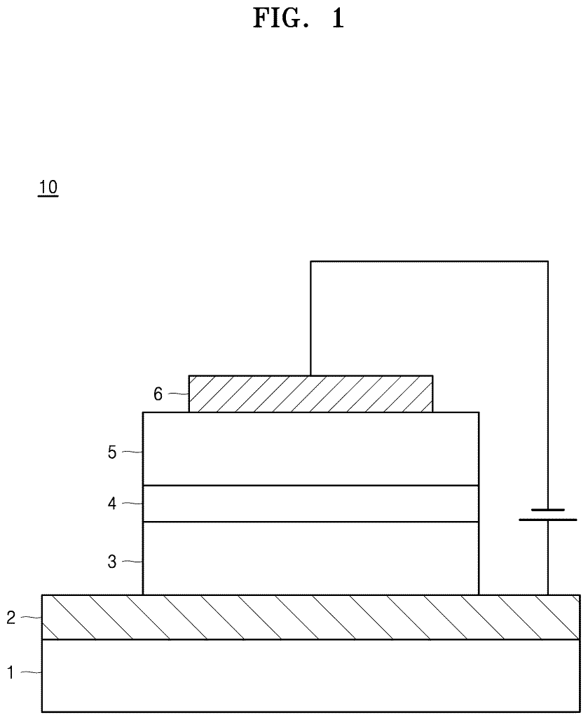

FIGS. 1 to 3 are each a schematic cross-sectional view illustrating an embodiment of an organic electroluminescent device. However, the structure of the organic electroluminescent device according to the disclosure is not limited to those shown in FIGS. 1 to 3.

FIG. 1 is a schematic cross-sectional view of an embodiment of an organic electroluminescent device. An organic electroluminescent device 10 according to an embodiment includes a substrate 1, a first electrode 2, a hole transport region 3, an emission layer 4, an electron transport region 5, and a second electrode 6, which are sequentially stacked in this stated order.

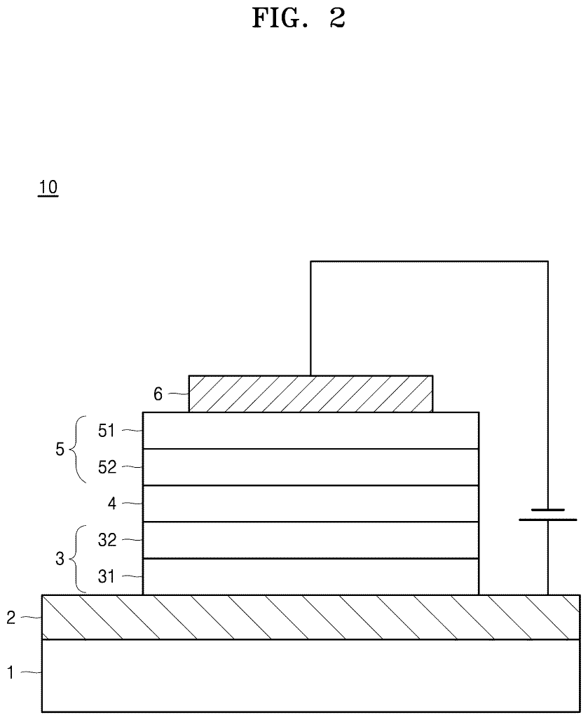

FIG. 2 is a schematic cross-sectional view of an embodiment of an organic electroluminescent device. The organic electroluminescent device 10 according to an embodiment includes the substrate 1, the first electrode 2, the hole transport region 3, the emission layer 4, the electron transport region 5, and the second electrode 6, which are sequentially stacked in this stated order. In FIG. 2, the hole transport region 3 includes a hole injection layer 31 and a hole transport layer 32, which are sequentially stacked in this stated order. In addition, in FIG. 2, the electron transport region 5 includes an electron transport layer 52 and an electron injection layer 51, which are sequentially stacked in this stated order.

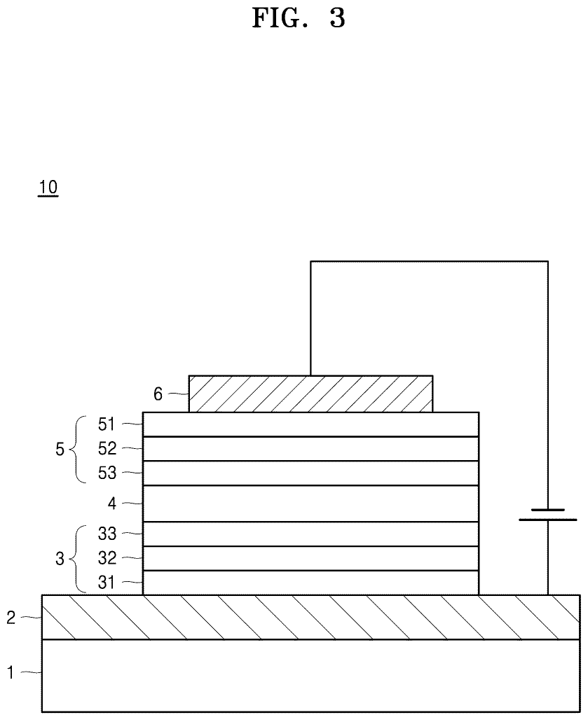

FIG. 3 is a schematic cross-sectional view of an embodiment of an organic electroluminescent device. The organic electroluminescent device 10 according to an embodiment includes the substrate 1, the first electrode 2, the hole transport region 3, the emission layer 4, the electron transport region 5, and the second electrode 6, which are sequentially stacked in this stated order. In FIG. 3, the hole transport region 3 includes the hole injection layer 31, the hole transport layer 32, and an electron blocking layer 33, which are sequentially stacked in this stated order. In addition, in FIG. 3, the electron transport region 5 includes a hole blocking layer 53, the electron transport layer 52, and the electron injection layer 51, which are sequentially stacked in this stated order.

Hereinafter, the substrate, each region, and each layer are described in detail.

### Substrate 1

The organic electroluminescent device 10 may have the substrate 1. As the substrate 1, any substrate used in general organic electroluminescent devices may be used. For example, the substrate 1 may be a glass substrate, a semiconductor substrate such as a silicon substrate, or a transparent plastic substrate.

### First Electrode 2

The first electrode 2 may be conductive. In the organic electroluminescent device 10 according to an embodiment, the first electrode 2 may be an anode. In addition, the first electrode 2 may be a pixel electrode. In addition, the first electrode 2 may be a transmissive electrode, a semi-transmissive electrode, or a reflective electrode.

A material for forming the first electrode 2 is not particularly limited, and may be, for example, a metal, a metal alloy, or a conductive compound. When the first electrode 2 is a transmissive electrode, the first electrode 2 may include a transparent metal oxide, for example, indium tin oxide (ITO), indium zinc oxide (IZO), zinc oxide (ZnO), indium tin zinc oxide (ITZO), or the like. In addition, when the first electrode 2 is a semi-transmissive electrode or a reflective electrode, the first electrode 2 may include Ag, Mg, Cu, Al, Pt, Pd, Au, Ni, Nd, Ir, Cr, Li, Ca, LiF/Ca, LiF/Al, Mo, Ti, or a compound or mixture thereof (for example, a mixture of Ag and Mg).

The first electrode 2 may be a single layer consisting of a single material or a single layer consisting of a plurality of different materials. In addition, the first electrode 2 may have a multilayer structure having a plurality of layers consisting of a plurality of different materials.

The thickness of the first electrode 2 is not particularly limited, but may be at least about 10 nm and not more than about 1,000 nm, for example, at least about 50 nm and not more than about 300 nm.

### Hole Transport Region 3

The hole transport region 3 is provided on the first electrode 2. The hole transport region 3 may include at least one of the hole injection layer 31, the hole transport layer 32, a hole buffer layer (not shown), and the electron blocking layer 33.

The hole transport region 3 may be a single layer consisting of a single material or a single layer consisting of a plurality of different materials. In addition, the hole transport region 3 may have a multilayer structure having a plurality of layers consisting of a plurality of different materials.

For example, the hole transport region 3 may have a single-layer structure including the hole injection layer 31 or the hole transport layer 32. In addition, for example, the hole transport region 3 may have a single-layer structure including a hole injection material and a hole transport material. In addition, for example, the hole transport region 3 may have a structure of the hole injection layer 31/the hole transport layer 32, wherein constituting layers are sequentially stacked from the first electrode 2. In addition, for example, the hole transport region 3 may have a structure of the hole injection layer 31/the hole transport layer 32/the hole buffer layer (not shown). In addition, for example, the hole transport region 3 may have a structure of the hole injection layer 31/the hole buffer layer (not shown), wherein constituting layers are sequentially stacked from the first electrode 2. In addition, for example, the hole transport region 3 may have a structure of the hole transport layer 32/the hole buffer layer (not shown), wherein constituting layers are sequentially stacked from the first electrode 2. In addition, for example, the hole transport region 3 may have a structure of the hole injection layer 31/the hole transport layer 32/the electron blocking layer 33, wherein constituting layers are sequentially stacked from the first electrode 2. However, the structure of the hole transport region is not limited to the examples above.

The hole injection layer 31 or each layer constituting the hole transport region 3 is not particularly limited, but may include, for example, a known hole injection material. Examples of the hole injection material may include a phthalocyanin compound such as copper phthalocyanin, N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino)triphenylamine (m-MTDATA), 4,4′,4″-tris(N,N-diphenylamino)triphenylamine (TDATA), 4,4′,4″-tris{N,-(2-naphthyl)-N-phenylamino}-triphenylamine (2-TNATA), poly(3,4-ethylenedioxythiophene)/poly(4-styrene sulfonate) (PEDOT/PSS), polyaniline/dodecylbenzenesulfonic acid (PANI/DBSA), polyaniline/camphorsulfonic acid (PANI/CSA), polyaniline/poly(4-styrenesulfonate) (PANI/PSS), N,N′-di(naphthalen-1-yl)-N,N′-diphenylbenzidine (NPB), polyetherketone including triphenylamine (TPAPEK), 4-isopropyl-4′-methyldiphenyliodoniumtetrakis(pentafluorophenyl)borate, dipyrazino[2,3-f:2′,3′-h]quinoxalin-2,3,6,7,10,11-hexacarbonitrile (HAT-CN), 1,3,4,5,7,8-hexafluorotetracyano-2,6-naphthoquinodimethane (F6-TCNNQ), or the like.

In addition, the hole transport layer 32 or each layer constituting the hole transport region 3 is not particularly limited, and may include, for example, a known hole transport material. Examples of the hole transport material may include a carbazole-based derivative such as N-phenylcarbazole, polyvinyl carbazole, or the like., a fluorene-based derivative, N,N′-bis(3-methylphenyl)-N,N′-diphenyl-[1,1-biphenyl]-4,4′-diamine (TPD), a triphenylamine-based derivative such as 4,4′,4″-tri(N-carbazolyl)triphenylamine (TCTA), N,N′-di(naphthalen-1-yl)-N,N′-diphenyl-benzidine (NPB), 4,4′-cyclohexylidenebis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), Compound HTM1, Compound HTM2, Compound HT3, or the like.

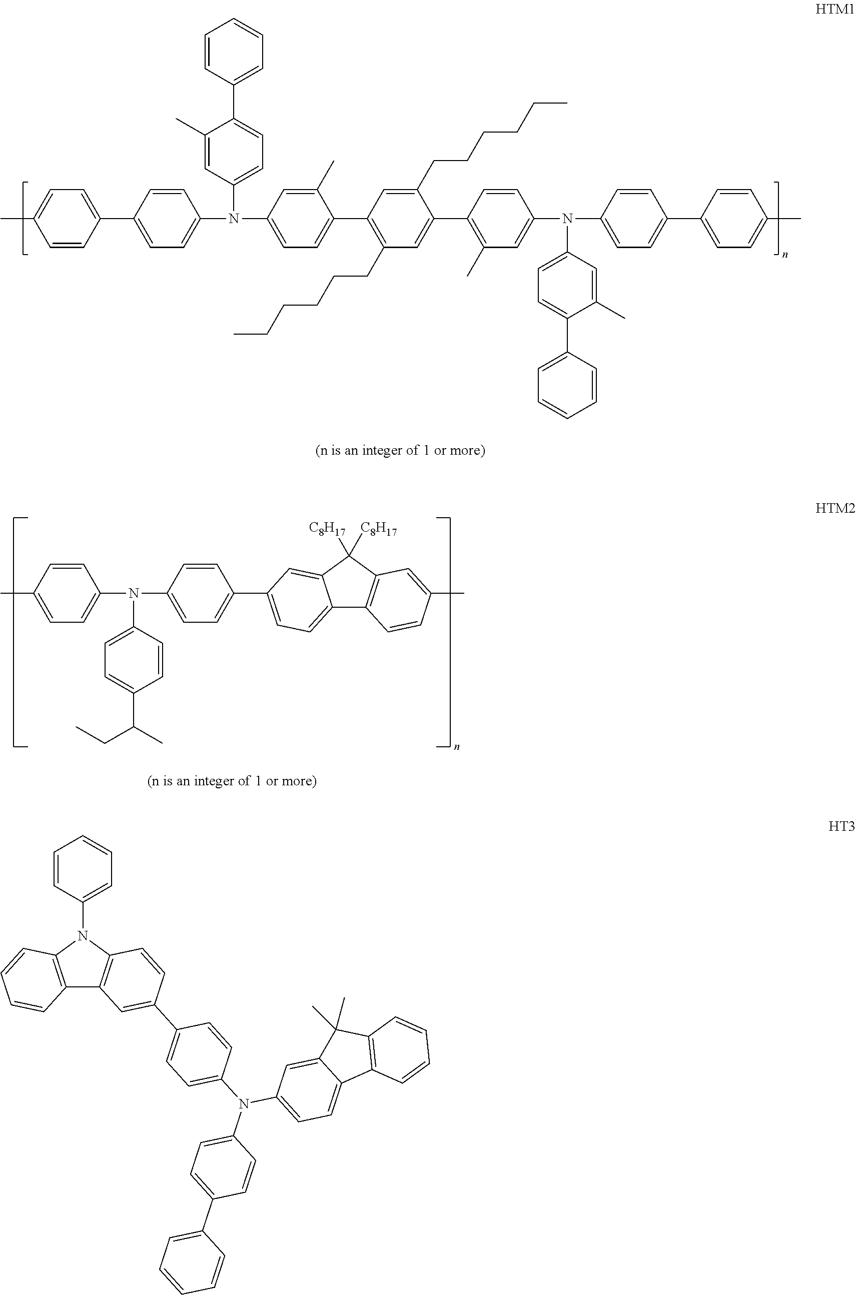

The hole transport region 3 may further include a charge generation material, in addition to the hole injection material or the hole transport material, to improve conductivity. The charge generation material may be homogeneously or non-homogeneously dispersed in the hole transport region 3 or each layer thereof. The charge generation material is not particularly limited, but examples thereof may include a known charge generation material. The charge generation material may be, for example, a p-dopant. Examples of the p-dopant may include a quinone derivative such as tetracyanoquinonedimethane (TCNQ), 2,3,5,6-tetrafluoro-tetracyanoquinodimethane (F4-TCNQ), or the like, a metal oxide such as tungsten oxide, molybdenum oxide, etc., a cyano group-containing compound, or the like.

The hole buffer layer (not shown) may compensate for an optical resonance distance according to a wavelength of light emitted from the emission layer 4 to improve photoluminescence emission efficiency. Materials included in the hole buffer layer (not shown) are not particularly limited, and materials used in a known hole buffer layer may be used. For example, the compounds that may be included in the hole transport region 3 may be used.

The electron blocking layer 33 may prevent injection of electrons from the electron transport region 5 to the hole transport region 3. Materials included in the electron blocking layer 33 are not particularly limited, and materials used in a known electron blocking layer may be used. For example, the host material, such as Compounds H55, H86, and H87, included in the emission layer (the material for an organic electroluminescent device) may be used.

The thickness of the hole transport region 3 is not particularly limited, but may be about 1 nm to about 1,000 nm, for example, about 10 nm to about 500 nm. In addition, regarding each layer constituting the hole transport region 3, the thickness of the hole injection layer 31 may be, but is not particularly limited to, about 3 nm to about 200 nm. The thickness of the hole transport layer 32 may be, but is not particularly limited to, about 3 nm to about 200 nm. The thickness of the electron blocking layer 33 may be, but is not particularly limited to, about 1 nm to about 100 nm. In addition, the thickness of the hole buffer layer (not shown) is not particularly limited as long as the hole buffer layer functions as a hole buffer layer and does not interfere with functions of an organic electroluminescent device. When the thickness of the hole transport region 3, the hole injection layer 31, the hole transport layer 32, or the electron blocking layer 33 is within the range above, excellent hole transport characteristics may be obtained while suppressing a substantial increase in driving voltage.

Examples of a film forming method of the hole transport region 3 or each layer thereof are not particularly limited, but may include known film forming methods such as vacuum deposition, spin coating, LB deposition, ink-jet printing, laser-printing, or LITI.

### Emission Layer 4

The emission layer 4 is disposed on the hole transport region 3. Details of the emission layer 4 are as described above.

### Electron Transport Region 5

The electron transport region 5 is disposed on the emission layer 4. The electron transport region 5 includes at least one of the electron injection layer 51, the electron transport layer 52, and the hole blocking layer 53, but embodiments are not limited thereto.

The electron transport region 5 may be a single layer consisting of a single material or a single layer consisting of a plurality of different materials. In addition, the electron transport region 5 may have a multilayer structure having a plurality of layers consisting of a plurality of different materials. For example, the electron transport region 5 may have a single-layer structure including the electron injection layer 51 or the electron transport layer 52. In addition, for example, the electron transport region 5 may have a single-layer structure consisting of an electron injection material and an electron transport material. In addition, for example, the electron transport region 5 may have a structure of the electron transport layer 52/the electron injection layer 51, wherein constituting layers are sequentially stacked from the emission layer 4. In addition, for example, the electron transport region 5 may have a structure of the hole blocking layer 53/the electron transport layer 52/the electron injection layer 51, wherein constituting layers are sequentially stacked from the emission layer 4. However, the structure of the electron transport region 5 is not limited to the examples above.

The electron injection layer 51 or each layer constituting the electron transport region 5 is not particularly limited, but may include, for example, a known electron injection material. Examples of the electron injection material may include a lanthanide metal such as LiF, lithium quinolate (LiQ), Li₂O, BaO, NaCl, CsF, or Yb, or a metal halide such as RbCl. The electron injection layer 51 is not particularly limited, and may include, for example, an electron transport material and an insulating organometallic salt. The organometallic salt is not particularly limited, and may be, for example, a material having an energy band gap of 4 electronvolts (eV) or more. The organometallic salt may be, for example, an acetate metallic salt, a benzoate metallic salt, an acetoacetate metallic salt, an acetylacetonate metallic salt, or a stearate metallic salt.

The electron transport layer 52 or each layer constituting the electron transport region 5 is not particularly limited, and may include, for example, a known electron transport material. Examples of the electron transport material may include an anthracene-based compound, tris(8-hydroxyquinolinato)aluminum (Alq₃), 1,3,5-tri[(3-pyridyl)-phen-3-yl]benzene, 2,4,6-tris(3′-pyridin-3-yl)biphenyl-3-yl)-1,3,5-triazine, 2-(4-(N-phenylbenzimidazolyl-1-ylphenyl)-9,10-dinaphthylanthracene, 1,3,5-tri(1-phenyl-1H-benzo[d]imidazol-2-yl)phenyl (TPBi), 2,9-dimethyl-4,7-diphenyl-1,10-phenanthroline (BCP), 4,7-diphenyl-1,10-phenanthroline (Bphen), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olato) (Bebq₂), 9,10-di(naphthalen-2-yl)anthracene (ADN), lithium quinolate (LiQ), Compound ET1, H91, or the like. In addition, TRE314 (product of Toray Co., Ltd., electron transport material) or the like may be used.

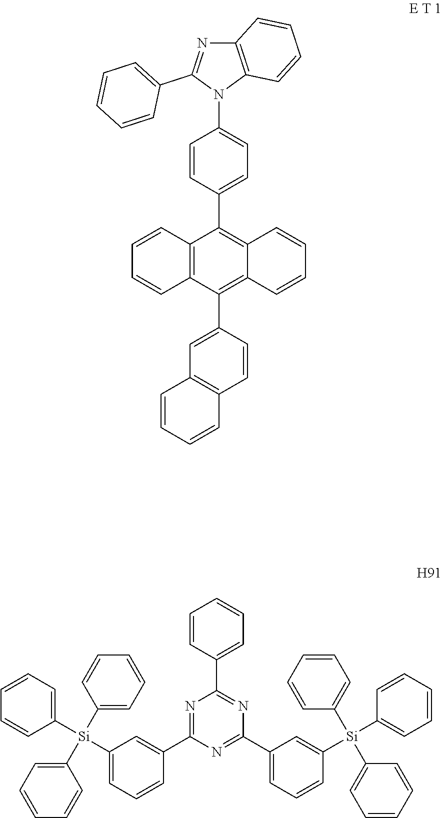 [SMILES: c1ccc(-c2nc3ccccc3n2-c2ccc(-c3c4ccccc4c(-c4ccc5ccccc5c4)c4ccccc34)cc2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1]

The hole blocking layer 53 may prevent injection of holes from the hole transport region 3 to the electron transport region 5. Materials included in the hole blocking layer 53 are not particularly limited, and materials used in a known hole blocking layer may be used. The hole blocking layer 53 may include, for example, a known hole blocking material. Examples of the hole blocking material may include 2,9-dimethyl-4,7-diphenyl-1,10-phenanthroline (BCP), 4,7-diphenyl-1,10-phenanthroline (BPhen), or the like. In addition, for example, the host material, such as Compounds H77 and H87, included in the emission layer (the material for an organic electroluminescent device) may be used.

The thickness of the electron transport region 5 is not particularly limited, but may be about 0.1 nm to about 200 nm, for example, about 30 nm to about 150 nm. In addition, regarding each layer constituting the electron transport region 5, the thickness of the electron transport layer 52 may be, but is not particularly limited to, about 10 nm to about 100 nm, for example, about 15 nm to about 500 nm. The thickness of the hole blocking layer 53 is not particularly limited, but may be about 1 nm to about 100 nm, for example, about 5 nm to about 30 nm. The thickness of the electron injection layer 51 is not particularly limited, but may be about 0.1 nm to about 10 nm, for example, about 0.3 nm to about 9 nm. When the thickness of the electron injection layer 51 is within the range above, excellent electron injection characteristics may be obtained while suppressing a substantial increase in driving voltage. In addition, when the thickness of the electron transport region 5, the electron injection layer 51, the electron transport layer 52, or the hole blocking layer 53 is within the range above, excellent hole transport characteristics may be obtained while suppressing a substantial increase in driving voltage.

Examples of a film forming method of the electron transport region 5 or each layer thereof are not particularly limited, but may include known film forming methods such as vacuum deposition, spin coating, LB deposition, ink-jet printing, laser-printing, or LITI.

### Second Electrode 6

The second electrode 6 is disposed on the electron transport region 5. The second electrode 6 is conductive. The second electrode 6 in the organic electroluminescent device according to an embodiment may be a common electrode or a cathode. In addition, the second electrode 6 may be a transmissive electrode, a semi-transmissive electrode, or a reflective electrode.

A material for forming the second electrode 6 is not particularly limited, but may be, for example, a metal, a metal alloy, or a conductive compound. When the second electrode 6 is a transmissive electrode, the second electrode 6 may include a transparent metal oxide, for example, ITO, IZO, ZnO, ITZO, or the like. When the second electrode 6 is a semi-transmissive electrode or a reflective electrode, the second electrode 6 may include Ag, Mg, Cu, Al, Pt, Pd, Au, Ni, Nd, Ir, Cr, Li, Ca, LiF/Ca, LiF/Al, Mo, Ti, or a compound or mixture thereof (for example, a mixture of Ag and Mg).

The second electrode 6 may be a single layer consisting of a single material or a single layer consisting of a plurality of different materials. In addition, the second electrode 6 may have a multilayer structure having a plurality of layers consisting of a plurality of different materials.

The thickness of the second electrode 6 may be, but is not particularly limited to, about 10 nm to about 1,000 nm.

The second electrode 6 may be connected to an auxiliary electrode (not shown). Because the second electrode 6 is connected to the auxiliary electrode, the resistance of the second electrode 6 may be further reduced.

In addition, a capping layer (not shown) may be further disposed on the second electrode 6. The capping layer (not shown) is not particularly limited, but may be, for example, a layer including α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tri-9-carbazolyltriphenylamine (TCTA), N,N′-bis(naphthalen-1-yl)-N,N′-bis(phenyl)benzidine (NPB), or the like.

In addition, materials constituting each layer and each electrode may be used alone or in combination of two or more.

In the organic electroluminescent device 10 of FIGS. 1 to 3, the compound of Formula (1) or the material for an organic electroluminescent device may be included in the emission layer 4, but may also be included in an organic layer other than the emission layer 4. In addition, the compound of Formula (1) or the material for an organic electroluminescent device may be included in the emission layer 4 and an organic layer other than the emission layer 4.

In the organic electroluminescent device 10 of FIGS. 1 to 3, since a voltage is applied to each of the first electrode 2 and the second electrode 6, holes provided from the first electrode 2 may move toward the emission layer 4 through the hole transport region 3, and electrons provided from the second electrode 6 may move toward the emission layer 4 through the electron transport region 5. The holes and the electrons may recombine in the emission layer 4 to produce excitons, and these excitons may transition from an excited state to a ground state to thereby generate light.

While embodiments of the disclosure have been described in detail, it will be understood that these embodiments are illustrative and not intended to be limiting, and the scope of the disclosure should be construed in accordance with the appended claims.

The disclosure includes the following aspects and embodiments.

Aspect 1. A compound represented by Formula (1):

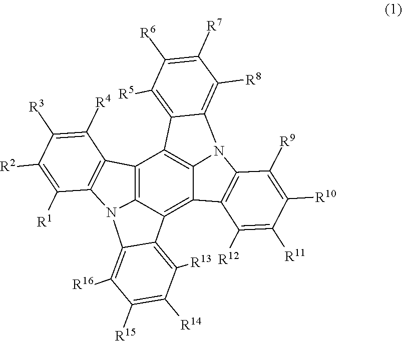

wherein, in Formula (1),
at least one of R¹ to R¹⁶ may be

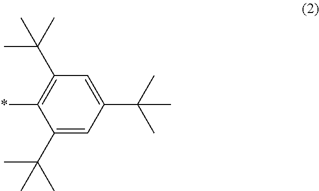 [SMILES: *c1c(C(C)(C)C)cc(C(C)(C)C)cc1C(C)(C)C]

a substituent W represented by Formula 2 (* indicates a binding site to a benzene ring), and
R¹ to R¹⁶ other than the substituent W may each independently be any one atom or group among (a1) to (a10):
(a1) a hydrogen or deuterium atom;
(a2) a halogen atom;
(a3) a cyano group;
(a4) a substituted or unsubstituted alkyl group having 1 to 20 carbon atoms;
(a5) a substituted or unsubstituted alkoxy group having 1 to 20 carbon atoms;
(a6) a substituted or unsubstituted arylamino group having 6 to 20 carbon atoms;
(a7) a substituted or unsubstituted triarylsilyl group, alkyldiarylsilyl group, dialkylarylsilyl group, or trialkylsilyl group (wherein an aryl group is an aryl group having 6 to 20 carbon atoms, and an alkyl group is an alkyl group having 1 to 20 carbon atoms);
(a8) a substituted or unsubstituted aromatic hydrocarbon group having 6 to 30 carbon atoms;
(a9) a substituted or unsubstituted heterocyclic group having 3 to 30 ring-forming atoms; and
(a10) a substituted or unsubstituted saturated hydrocarbon group or saturated heterocyclic group, each formed by bonding two adjacent groups among R¹ to R¹⁶ and having 5 to 9 ring-forming atoms.

Aspect 2. The compound of aspect 1, wherein at least two of R², R³, R⁶, R⁷, R¹⁰, R¹¹, R¹⁴, and R¹⁵, may each be the substituent W.

Aspect 3. An organic electroluminescent device having an emission layer including the compound of aspect 1 or 2.

Aspect 4. The organic electroluminescent device of aspect 3, wherein the emission layer further includes a phosphorescent complex.

### EXAMPLES

Hereinafter, the disclosure will be described in more detail with reference to the following Examples and Comparative Examples, but the technical scope of the disclosure is not limited thereto.

### Synthesis of Compound

Compounds 1 to 4 were synthesized according to the following Synthetic Examples for use in the manufacture of organic electroluminescent devices. In addition, Comparative Compounds C1 to C4 were prepared for use in the manufacture of comparative organic electroluminescent devices.

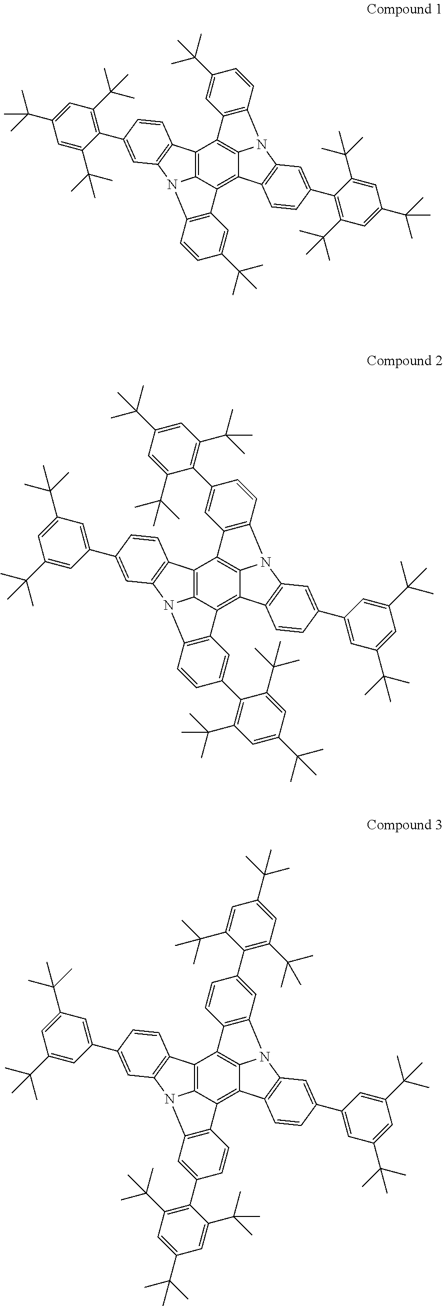

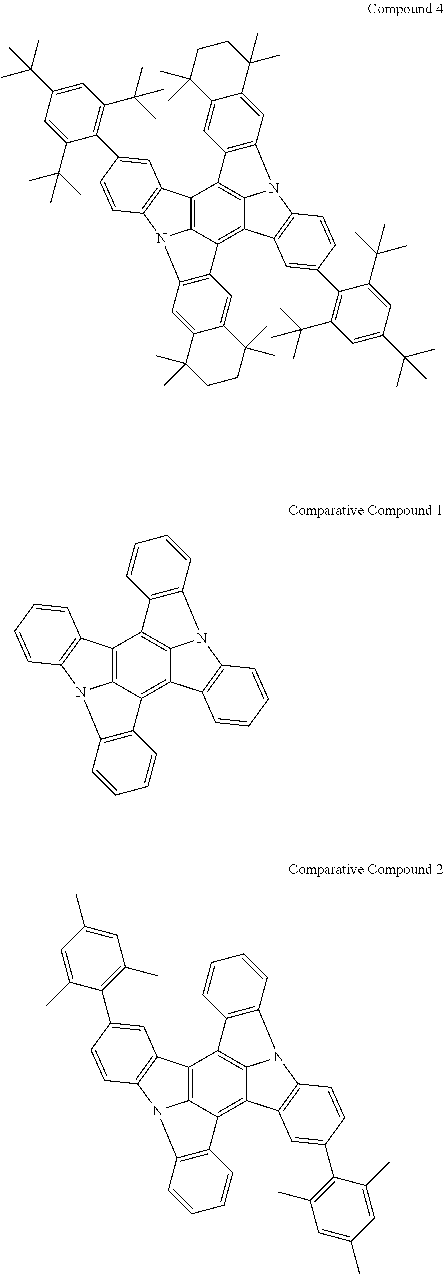

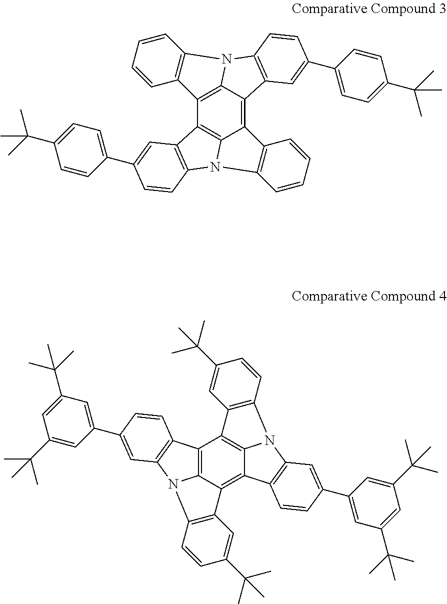

### Synthesis Example 1

Compound 1 was synthesized according to the following scheme.

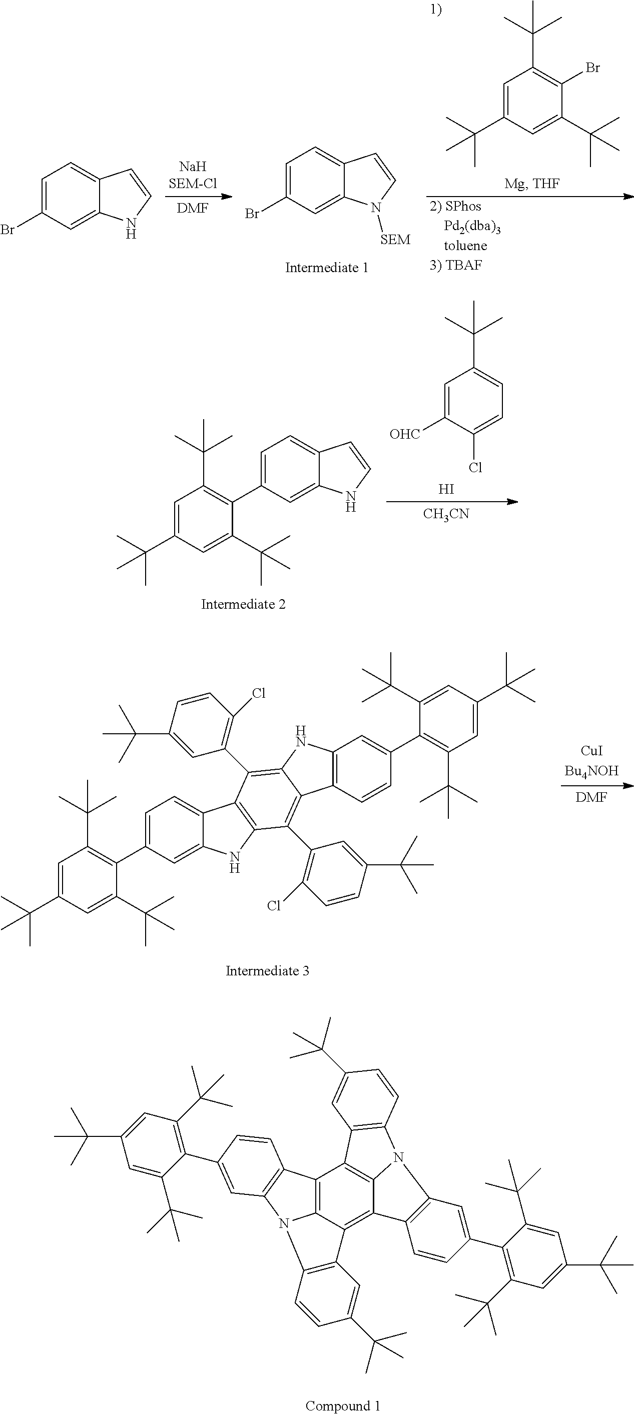

### Synthesis of Intermediate 1

Under a nitrogen atmosphere, 6-bromoindole (15 grams (g), 76.5 millimoles (mmol)) and N,N-dimethylformamide (DMF) (600 milliliters (mL)) were combined in a 1 L 3-neck flask and then cooled to 0° C. Sodium hydride (3.7 g, 91.8 mmol) (60 wt % of sodium hydride dispersed in liquid paraffin) was added in three portions, and the obtained reaction solution was stirred at 0° C. for 1 hour. Afterwards, the reaction solution was heated to room temperature, stirred for 1 hour, and then cooled to 0° C. After cooling, 2-(chloromethoxy)ethyltrimethylsilane (“SEM-Cl” in the above scheme) (15.3 g, 91.8 mmol) was slowly added dropwise to the reaction solution and stirred at room temperature for 12 hours. Next, a water suspension obtained by injecting the reaction solution into ice was extracted with hexane (200 mL×3). The extracted organic layer was washed with a saturated saline solution (200 mL) and then dried over anhydrous sodium sulfate (desiccant). After filtration, solvent in the organic layer was removed by distillation under reduced pressure. An obtained residue was purified by silica gel column chromatography (eluent; hexane) to provide Intermediate 1 as a colorless liquid (25.5 g, 85% yield). In addition, in the above scheme, “SEM” refer to “(CH₃)₃SiCH₂CH₂OCH₂”.

### Synthesis of Intermediate 2

Under a nitrogen atmosphere, magnesium (3.74 g, 153 mmol) and anhydrous tetrahydrofuran (THF) (150 mL) were combined in a 1 L 3-neck flask, and a small amount of 1,2-dibromoethane was added thereto and then stirred at room temperature for 30 minutes. A solution of 2,4,6-tri-tert-butylbromobenzene (25 g, 76.9 mmol) in anhydrous tetrahydrofuran (150 mL) was slowly added dropwise to the reaction solution and then stirred at 60° C. for 2 hours to prepare a Grignard reagent solution. Intermediate 1 (12.5 g, 38.4 mmol), tris(dibenzylideneacetone)dipalladium(0) (in the above scheme, “Pd₂(dba)₃”) (1.76 g, 1.92 mmol), 2-dicyclohexylphosphino-2′-6′-dimethoxybiphenyl (in the above scheme, “SPhos”) (1.58 g, 3.84 mmol), and toluene (100 mL) were added to another 1 L 3-neck flask and then stirred. The Grignard reagent solution prepared above was added dropwise to this solution at room temperature, and the resulting reaction solution was stirred at 70° C. for 10 hours. Afterwards, the reaction solution was cooled to room temperature, and insoluble components in the reaction solution were removed by filtration using Celite. The organic layer of the reaction solution was washed with a saturated saline solution (200 mL×2) and then dried over anhydrous sodium sulfate (desiccant). After filtration, solvent in the organic layer was removed by distillation under reduced pressure. The obtained residue was dissolved in DMF (100 mL), and tetrabutylammonium fluoride hydrate (30.1 g, 115 mmol) and ethylenediamine (6.92 g, 115 mmol) were added to this solution and stirred at 100° C. for 6 hours. The resulting reaction solution was cooled to room temperature, the reaction solution was added to 200 mL of water, and this solution was extracted with ethyl acetate (200 mL×3). The extracted organic layer was washed with saturated saline solution (300 mL) and then dried over anhydrous sodium sulfate (desiccant). After filtration, solvent in the organic layer was removed by distillation under reduced pressure. The obtained residue was purified by silica gel column chromatography (eluent; hexane:ethyl acetate (volume ratio=95:5) to provide Intermediate 2 as a white solid (7.0 g, 50% yield).

### Synthesis of Intermediate 3

Under a nitrogen atmosphere, 5-tert-butyl-2-chlorobenzaldehyde (2.2 g, 11.1 mmol), Intermediate 2 (4.0 g, 11.1 mmol), and 110.6 mL of acetonitrile were combined in a 100 mL 3-neck flask. The resulting reaction solution was heated to 80° C., and 57 wt % hydroiodic acid (0.3 mL, 2.2 mmol) was added thereto and stirred for 2 hours. After the reaction solution was cooled to room temperature, precipitated solids were filtered off, and the obtained solids were washed with cooled acetonitrile to provide Intermediate 3 as a white powder (4.3 g, 73% yield).

### Synthesis of Compound 1

Under a nitrogen atmosphere, Intermediate 3 (4.3 g, 4.1 mmol), tetrabutylammonium hydroxide (37 wt % methanol solution) (17.2 mL, 20.3 mmol), copper(I) iodide (3.9 g, 20.3 mmol), and 41 mL of dimethylformamide were combined in a 100 mL 3-neck flask, and the resulting reaction solution was heated and stirred at 140° C. The reaction was allowed to proceed for 24 hours while tetrabutylammonium hydroxide (37 wt % methanol solution) (17.2 mL, 20.3 mmol) and copper(I) iodide (3.9 g, 20.3 mmol) were added to the reaction solution every 8 hours. Afterwards, the reaction solution was cooled to room temperature, and precipitated solids were filtered off. The filtered-off solids, 200 ml of methanol, and 50 mL of ethylenediamine were combined in a 500 mL triangular flask and stirred, and solids were filtered out and extracted. The solids thus obtained were dispersed and washed with THF-acetone to provide Compound 1 as a yellow solid (1.5 g, 35% yield). LC-MS: 1005([M+H]⁺.

### Compound Data (Identification Data) of Compound 1

The structure of the obtained Compound 1 was identified by nuclear magnetic resonance spectroscopy:

¹H NMR data (400 MHz, CD₂Cl₂) (parts per million, ppm) 1.24 (36H, s), 1.47 (18H, s), 1.85 (18H, s), 7.61 (2H, dd, J=7.8, 1.2 Hz), 7.69 (4H, s), 7.76 (2H, dd, J=8.7, 1.8 Hz), 8.03 (2H, d, J=8.7 Hz), 8.14 (2H, d, J=1.2 Hz), 8.55 (2H, d, J=7.8 Hz), 8.69 (2H, d, J=1.8 Hz).

### Synthesis Example 2

Compound 2 was synthesized according to the following scheme.

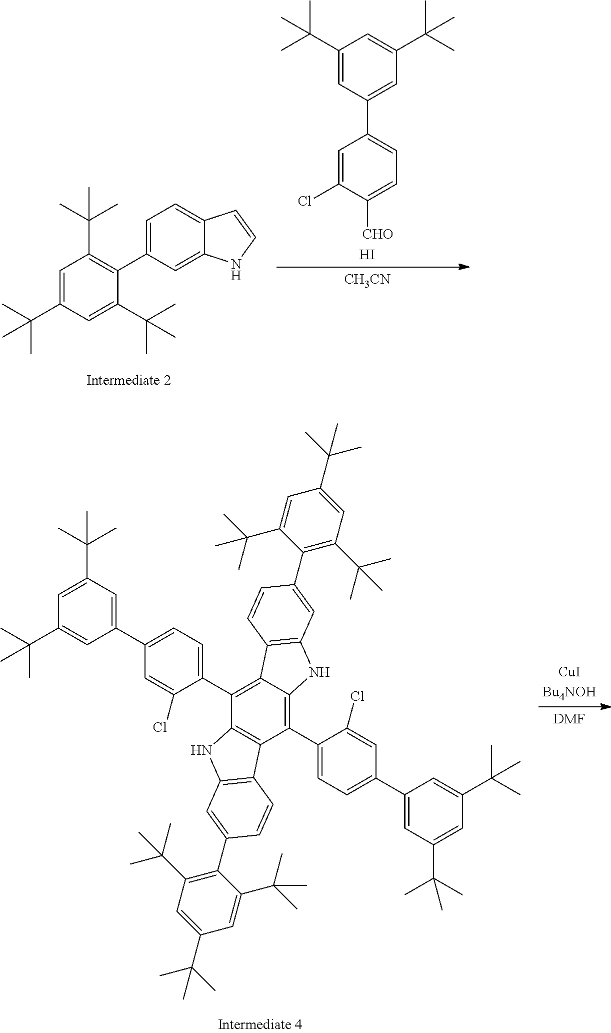

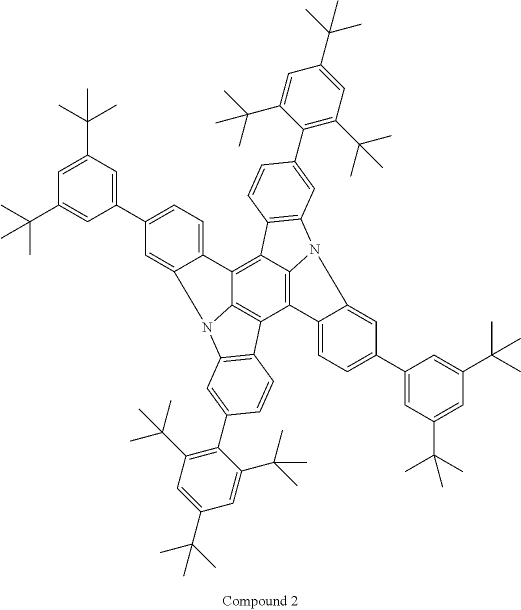 [SMILES: CC(C)(C)c1cc(-c2ccc3c4c5c6ccc(-c7c(C(C)(C)C)cc(C(C)(C)C)cc7C(C)(C)C)cc6n6c7cc(-c8cc(C(C)(C)C)cc(C(C)(C)C)c8)ccc7c(c7c8ccc(-c9c(C(C)(C)C)cc(C(C)(C)C)cc9C(C)(C)C)cc8n(c3c2)c47)c56)cc(C(C)(C)C)c1]

### Synthesis of Intermediate 4

Intermediate 4 was synthesized in the same manner as Intermediate 3. Intermediate 2 (3.96 g, 10.95 mmol) and 3′,5′-di-(tert-butyl)-3-chloro-4-formyl-[1,1′-biphenyl](3.6 g, 10.95 mmol) were used to obtain Intermediate 4 (4.64 g, 63% yield).

### Synthesis of Compound 2

Compound 2 was synthesized in the same manner as Compound 1. Intermediate 4 (2.32 g, 1.73 mmol) was used for the synthesis to obtain Compound 2 as a yellow solid (1.75 g, 80% yield). LC-MS: 1269([M]⁺.

### Synthesis Example 3

Compound 3 was synthesized according to the following scheme.

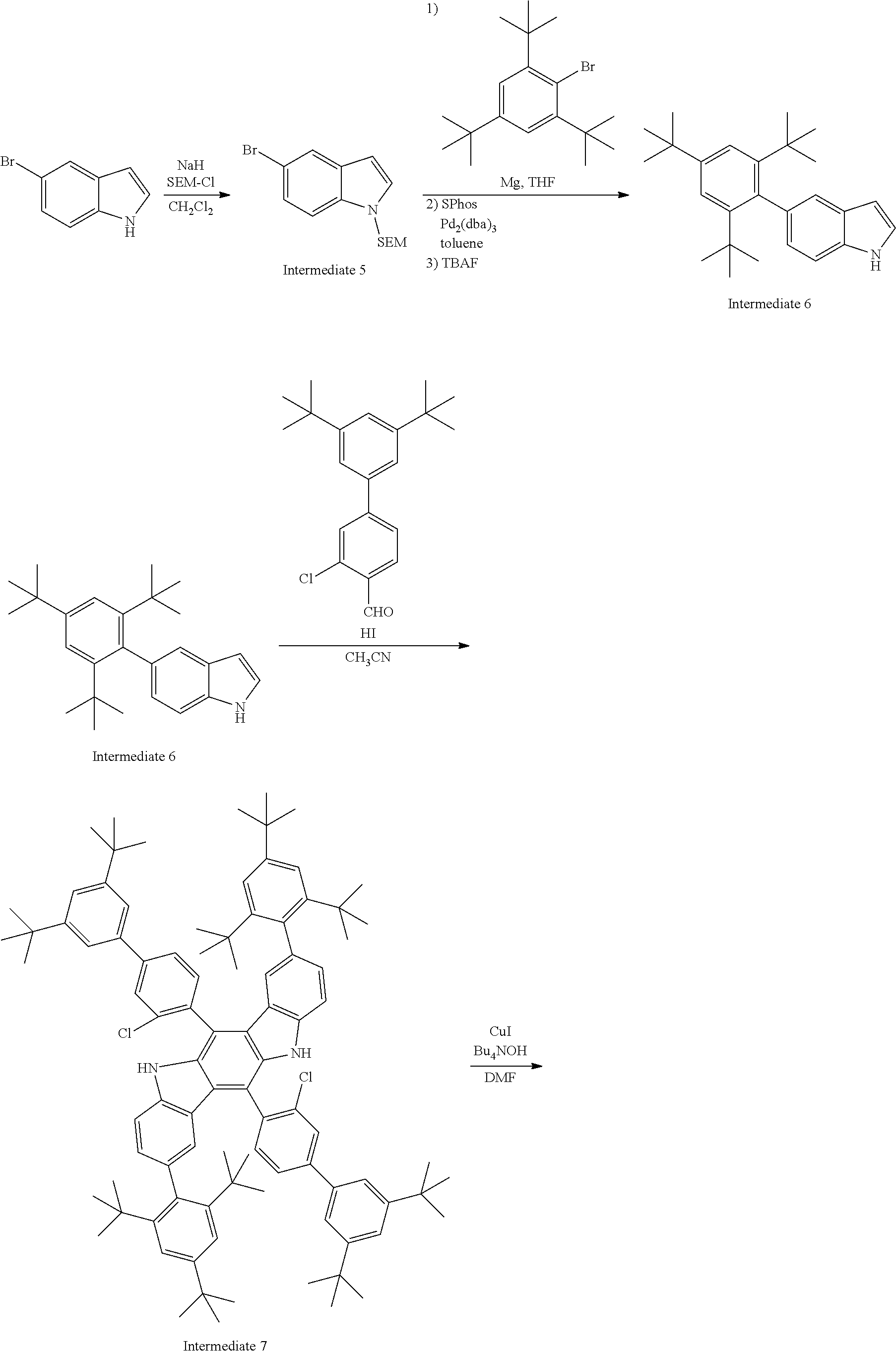

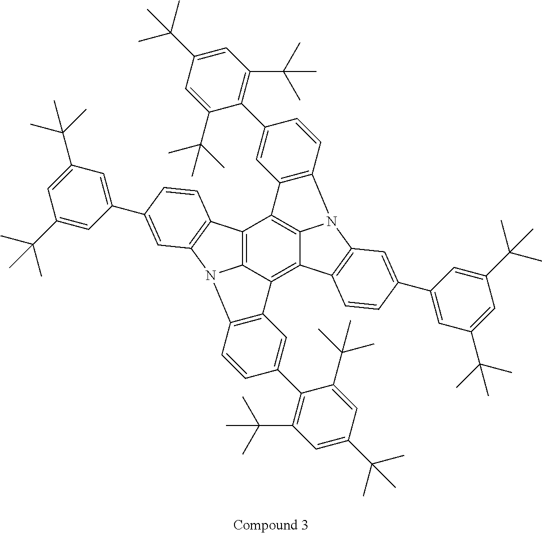 [SMILES: CC(C)(C)c1cc(-c2ccc3c4c5c6cc(-c7c(C(C)(C)C)cc(C(C)(C)C)cc7C(C)(C)C)ccc6n6c7cc(-c8cc(C(C)(C)C)cc(C(C)(C)C)c8)ccc7c(c7c8cc(-c9c(C(C)(C)C)cc(C(C)(C)C)cc9C(C)(C)C)ccc8n(c3c2)c74)c56)cc(C(C)(C)C)c1]

### Synthesis of Intermediate 5

Intermediate 5 was synthesized in the same manner as for Intermediate 1. 5-bromoindole (15 g, 76.5 mmol) was used to obtain Intermediate 5 as a colorless liquid (27.5 g, 85% yield).

### Synthesis of Intermediate 6

Intermediate 6 was synthesized in the same manner as Intermediate 2. Intermediate 5 (25 g, 127 mmol) was used to obtain Intermediate 6 as a white solid (7.0 g, 30% yield).

### Synthesis of Intermediate 7

Intermediate 7 was synthesized in the same manner as Intermediate 3. Intermediate 6 (3.96 g, 10.95 mmol) and 3′,5′-di-(tert-butyl)-3-chloro-4-formyl-[1,1′-biphenyl](3.6 g, 10.95 mmol) were used to obtain Intermediate 7 (3.1 g, 43% yield).

### Synthesis of Compound 3

Compound 3 was synthesized in the same manner as Compound 1. Intermediate 7 (2.12 g, 1.58 mmol) was used for the synthesis to obtain Compound 3 as a yellow solid (1.2 g, 60% yield). LC-MS: 1269([M]⁺.

### Synthesis Example 4

Compound 4 was synthesized according to the following scheme.

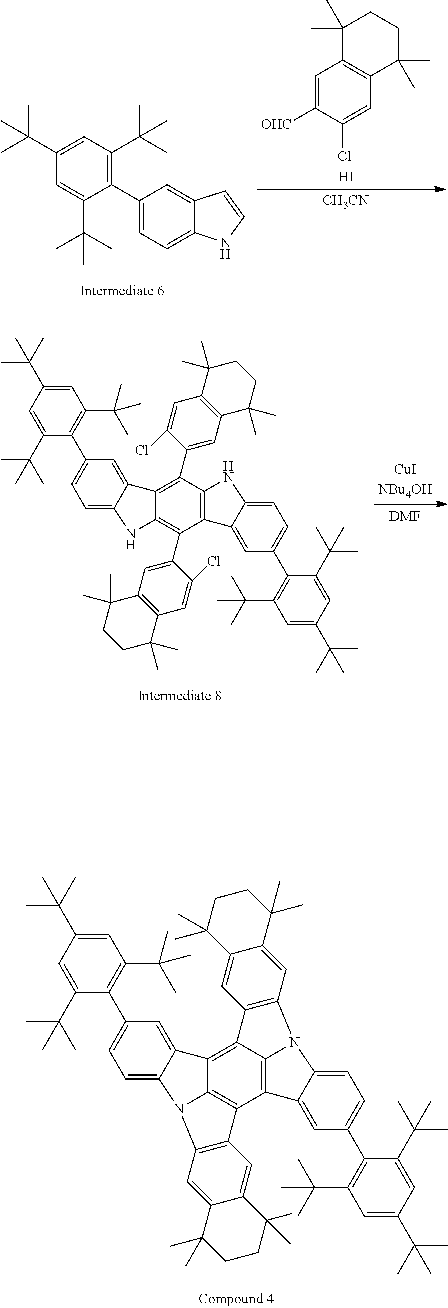

### Synthesis of Intermediate 8

Intermediate 8 was synthesized in the same manner as Intermediate 3. Intermediate 6 (2.7 g, 7.6 mmol) and 3-chloro-5,5,8,8-tetramethyl-2-formyl-5,6,7,8-tetrahydronaphthalene (1.9 g, 7.6 mmol) were used to obtain Intermediate 8 (3.6 g, 40% yield).

### Synthesis of Compound 4

Compound 4 was synthesized in the same manner as Compound 1. Intermediate 8 (3.0 g, 2.5 mmol) was used for the reaction to obtain Compound 4 as a yellow solid (1.9 g, 68% yield). LC-MS: 1114([M+H]⁺.

### Emission Spectrum of Solution

### Measurement Method

A fluorescence spectrum of photoluminescence (PL) was obtained from a toluene solution with a concentration of 1×10⁻⁷ M of the compound at room temperature at an excitation wavelength of 320 nm using a spectrofluorometer F-7000 manufactured by Hitachi High-Tech Co., Ltd. A peak wavelength and an emission spectrum width were determined from the measured emission spectrum. A wavelength representing the maximum value in the emission spectrum is defined as a “peak emission wavelength,” a wavelength width corresponding to half of the maximum value is defined as a “a full width at half maximum (FWHM),” and a wavelength width corresponding to one quarter of the maximum value is defined as a “full width at quarter maximum (FWQM).”

In addition, in this evaluation, the peak emission wavelength is not particularly limited, but may be within a blue emission region, and may be about 455 nm to about 475 nm.

In this evaluation, it is considered that the smaller the emission spectrum width (FWHM and FWQM), the better the color purity.

Table 1 provides peak emission wavelengths (nm) and emission spectrum width (FWHM and FWQM) of emission spectra of Compounds 1 to 4 and Comparative Compound C1, which were each in a toluene solution for the above described measurement method.

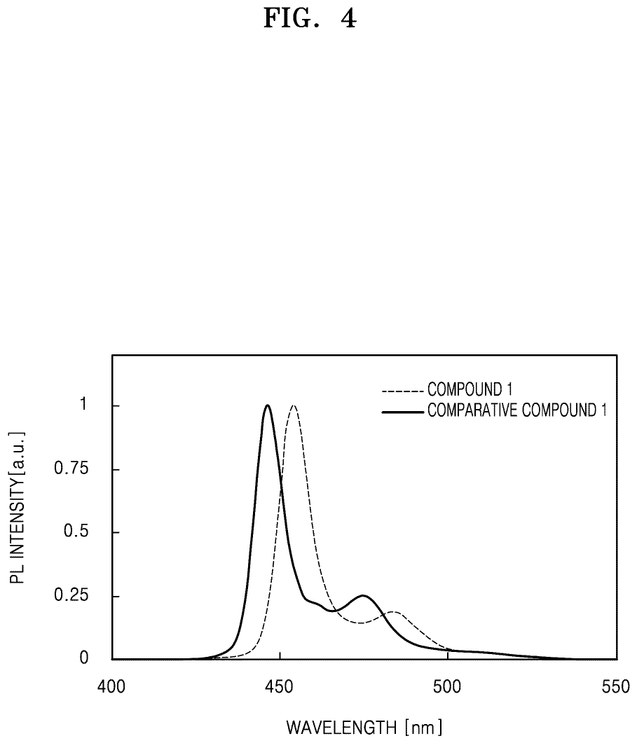

In addition, the emission spectra of Compound 1 and Comparative Compound C1 in a toluene solution for the above described measurement method are shown in FIG. 4.

TABLE 1
Emission spectrum in toluene solution

| | | Peak | | |
|---|---|---|---|---|
| | | emission | | |
| | | wavelength | FWHM | FWQM |
| Compound | Chemical structure | [nm] | [nm] | [nm] |
| Compound 1 | 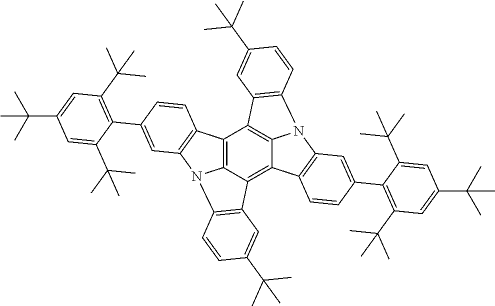 [SMILES: CC(C)(C)c1cc(C(C)(C)C)c(-c2ccc3c4c5c6cc(C(C)(C)C)ccc6n6c7cc(-c8c(C(C)(C)C)cc(C(C)(C)C)cc8C(C)(C)C)ccc7c(c7c8cc(C(C)(C)C)ccc8n(c3c2)c74)c56)c(C(C)(C)C)c1] | 454 | 11 | 17 |
| Compound 2 | 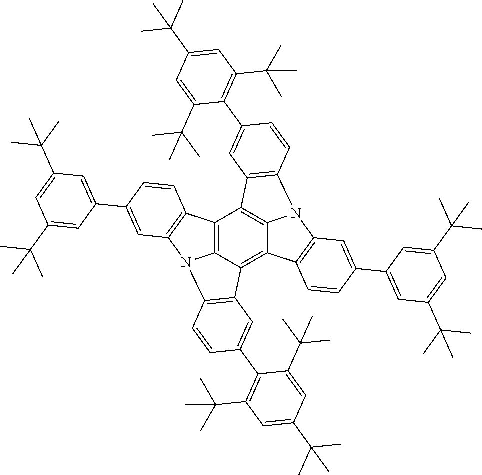 [SMILES: CC(C)(C)c1cc(-c2ccc3c4c5c6cc(-c7c(C(C)(C)C)cc(C(C)(C)C)cc7C(C)(C)C)ccc6n6c7cc(-c8cc(C(C)(C)C)cc(C(C)(C)C)c8)ccc7c(c7c8cc(-c9c(C(C)(C)C)cc(C(C)(C)C)cc9C(C)(C)C)ccc8n(c3c2)c74)c56)cc(C(C)(C)C)c1] | 460 | 13 | 21 |
| Compound 3 | 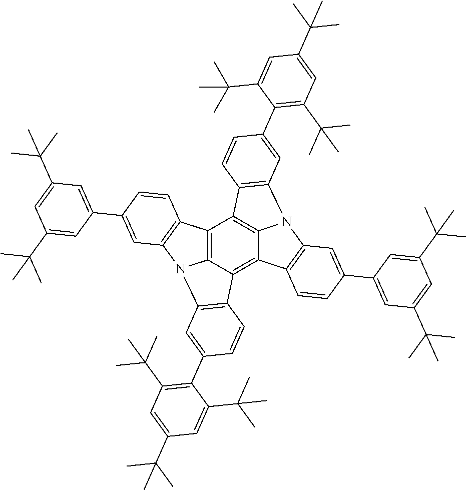 [SMILES: CC(C)(C)c1cc(-c2ccc3c4c5c6ccc(-c7c(C(C)(C)C)cc(C(C)(C)C)cc7C(C)(C)C)cc6n6c7cc(-c8cc(C(C)(C)C)cc(C(C)(C)C)c8)ccc7c(c7c8ccc(-c9c(C(C)(C)C)cc(C(C)(C)C)cc9C(C)(C)C)cc8n(c3c2)c47)c56)cc(C(C)(C)C)c1] | 459 | 14 | 22 |
| Compound 4 | 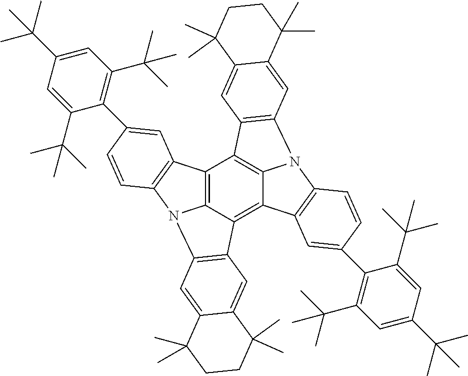 [SMILES: CC(C)(C)c1cc(C(C)(C)C)c(-c2ccc3c(c2)c2c4c5cc6c(cc5n5c7ccc(-c8c(C(C)(C)C)cc(C(C)(C)C)cc8C(C)(C)C)cc7c(c7c8cc9c(cc8n3c27)C(C)(C)CCC9(C)C)c45)C(C)(C)CCC6(C)C)c(C(C)(C)C)c1] | 456 | 12 | 19 |
| Comparative Compound C1 | 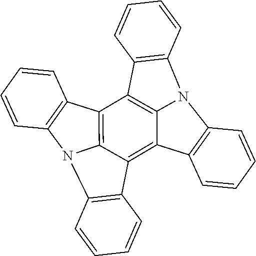 [SMILES: c1ccc2c(c1)c1c3c4ccccc4n4c5ccccc5c(c5c6ccccc6n2c15)c34] | 446 | 12 | 35 |

As shown in Table 1, the peak emission wavelengths of Compounds 1 to 4 of the disclosure were 454 nm to 460 nm, indicating blue emission. In addition, the FWQMs of Compounds 1 to 4 were 17 nm to 22 nm, which were 13 nm to 18 nm less than Comparative Compound C1. These results are believed to be due to the intensity of a secondary emission peak being suppressed by the effect of the substituent W (tri-tert-butylphenyl group) introduced into Compounds 1 to 4. As shown in the emission spectra of FIG. 4, the peak of the emission spectrum of Compound 1 of the disclosure had a longer wavelength than Comparative Compound 1, making it suitable for blue emission.

### Properties of Thin Films

### Method of Manufacturing Thin Films

Compounds shown in Tables 2 and 3 were co-deposited on a quartz substrate at a weight ratio of 1 wt % with respect to a host compound at a vacuum degree of 10⁻⁵ pascals (Pa) to manufacture a thin film (hereinafter, referred to as a “host dispersion film”) having a thickness of 50 nm. Compound HT1 and Compound HT2 were used as the host compound, and the weight ratio was set to Compound HT1:Compound HT2=60:40. In addition, the structures of HT1 and HT2 are as follows.

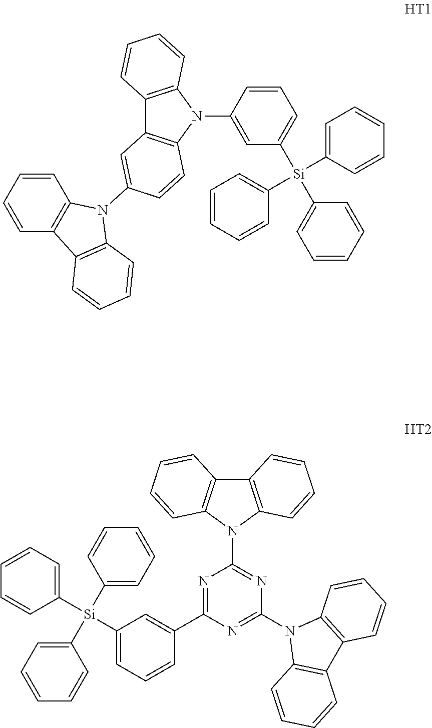 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

### Measurement (FWHM) of Photoluminescence (PL)

Thin films (host dispersion films) manufactured using the compounds in Table 2 were cut into a strip shape having a width of 6 millimeter (mm) and were subjected to PL measurement at room temperature using the spectrofluorometer F-7000 manufactured by Hitachi High-Tech Co., Ltd. From the obtained emission spectra, peak wavelengths (maximum emission wavelengths) and wavelength widths (FWHM) at which the emission intensity was halved were calculated. These evaluation results are shown in Table 2.

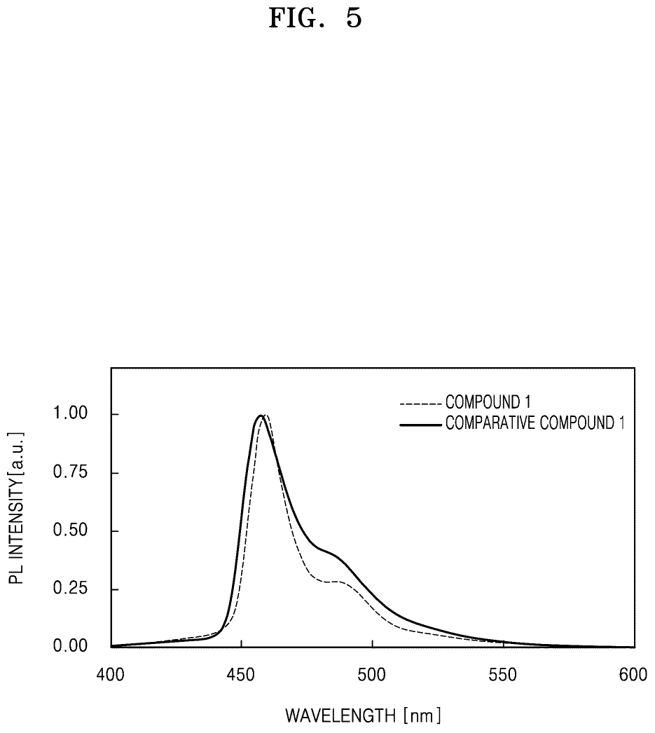

In addition, the emission spectra of the thin films (host dispersion films) manufactured using Compound 1 and Comparative Compound C1 are shown in FIG. 5.

TABLE 2
Emission spectrum in host dispersion film

| | | Peak | |
|---|---|---|---|
| | | emission | |
| | | wavelength | FWHM |
| Compound | Chemical structure | [nm] | [nm] |
| Compound 1 | 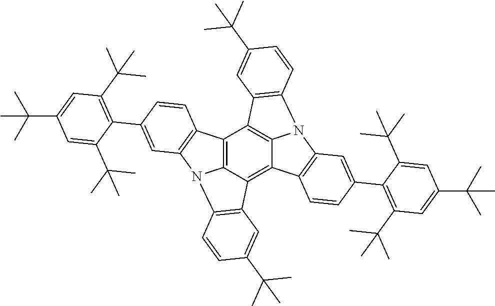 [SMILES: CC(C)(C)c1cc(C(C)(C)C)c(-c2ccc3c4c5c6cc(C(C)(C)C)ccc6n6c7cc(-c8c(C(C)(C)C)cc(C(C)(C)C)cc8C(C)(C)C)ccc7c(c7c8cc(C(C)(C)C)ccc8n(c3c2)c74)c56)c(C(C)(C)C)c1] | 459 | 17 |
| Compound 2 | 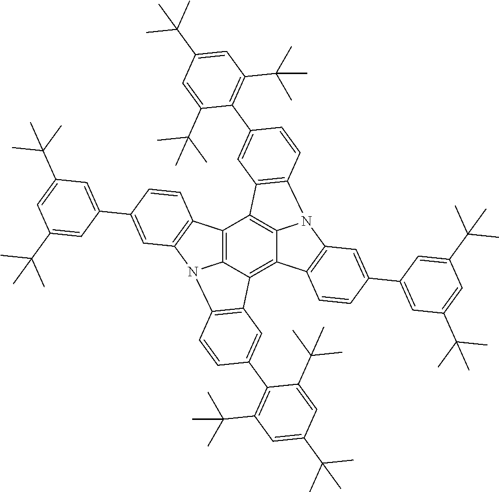 [SMILES: CC(C)(C)c1cc(-c2ccc3c4c5c6cc(-c7c(C(C)(C)C)cc(C(C)(C)C)cc7C(C)(C)C)ccc6n6c7cc(-c8cc(C(C)(C)C)cc(C(C)(C)C)c8)ccc7c(c7c8cc(-c9c(C(C)(C)C)cc(C(C)(C)C)cc9C(C)(C)C)ccc8n(c3c2)c74)c56)cc(C(C)(C)C)c1] | 466 | 19 |
| Comparative Compound C1 | 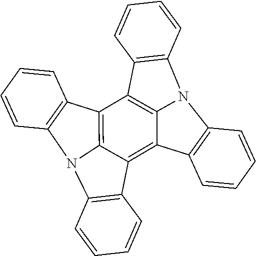 [SMILES: c1ccc2c(c1)c1c3c4ccccc4n4c5ccccc5c(c5c6ccccc6n2c15)c34] | 457 | 24 |

The emission peak wavelengths of compounds 1 and 2 of the thin films were respectively 459 nm and 466 nm, indicating blue emission as with the emission spectra for the toluene solutions. In addition, the FWHMs of the thin film emission spectra of Compounds 1 and 2 of the disclosure were respectively 17 nm and 19 nm, and had smaller differences from the FWHMs of the emission spectra in the toluene solutions than the difference for the FWHM of Comparative Compound C1 in the toluene solution versus the thin film. These results are believed to be due to the sterically bulky substituent W, which was newly introduced, effectively inhibiting intermolecular aggregation in a host-dispersed state, Compounds 1 and 2 of the disclosure were able to reduce the increase in FWHM. Meanwhile, Comparative Compound C1 had an emission peak wavelength of 457 nm, indicating blue emission, a large FWHM of 24 nm, and a different spectral shape for the thin film than the emission spectrum in the toluene solution. The observed effects are assumed to be because Comparative Compound C1 is a planar molecule leading to aggregation between molecules of Comparative Compounds C1, resulting in an increase in FWHM.

### Measurement of Photoluminescence Quantum Yield (PLQY)

For thin films (host dispersion films) manufactured using the compounds in Table 3, PLQYs were measured using a Quantaurus-QY absolute PLQY measurement device C11347-01 manufactured by Hamamatsu Photonics Co., Ltd. The excitation wavelength was measured by scanning at intervals of 10 nm from 280 nm to 350 nm, and the excitation wavelength region in which the compound absorption value provided 20% or more of the excitation light intensity ratio was adopted. The value of PLQY was taken as the highest value in the adopted excitation wavelength region. These evaluation results are shown in Table 3.

TABLE 3
Quantum yield in host dispersion film

| Compound | Chemical structure | PLQY [%] |
|---|---|---|
| Compound 1 | 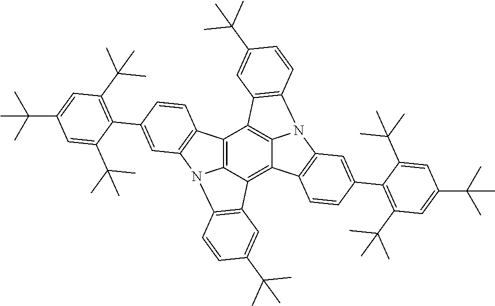 [SMILES: CC(C)(C)c1cc(C(C)(C)C)c(-c2ccc3c4c5c6cc(C(C)(C)C)ccc6n6c7cc(-c8c(C(C)(C)C)cc(C(C)(C)C)cc8C(C)(C)C)ccc7c(c7c8cc(C(C)(C)C)ccc8n(c3c2)c74)c56)c(C(C)(C)C)c1] | 78 |
| Comparative Compound C1 | 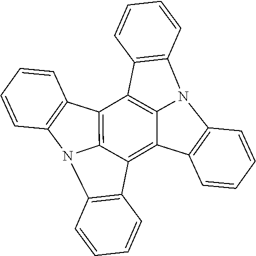 [SMILES: c1ccc2c(c1)c1c3c4ccccc4n4c5ccccc5c(c5c6ccccc6n2c15)c34] | 65 |
| Comparative Compound C2 | 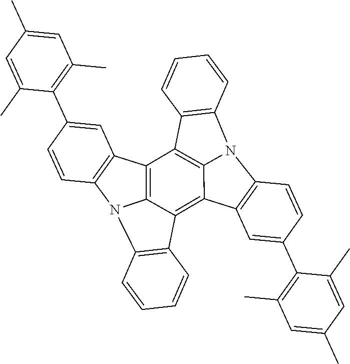 | 66 |
| Comparative Compound C3 | 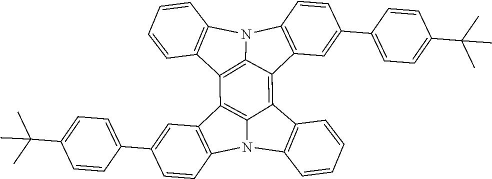 [SMILES: CC(C)(C)c1ccc(-c2ccc3c(c2)c2c4c5ccccc5n5c6ccc(-c7ccc(C(C)(C)C)cc7)cc6c(c6c7ccccc7n3c62)c45)cc1] | 68 |
| Comparative Compound C4 | 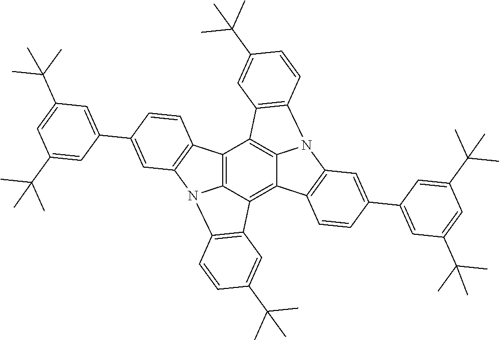 [SMILES: CC(C)(C)c1cc(-c2ccc3c4c5c6cc(C(C)(C)C)ccc6n6c7cc(-c8cc(C(C)(C)C)cc(C(C)(C)C)c8)ccc7c(c7c8cc(C(C)(C)C)ccc8n(c3c2)c74)c56)cc(C(C)(C)C)c1] | 71 |

As shown in Table 3, Compound 1 of the disclosure had a PLOY that is 11% greater than that of Comparative Compound C1. In addition, it may be confirmed that Compound 1 had a PLOY that is significantly greater than that of Comparative Compound C4 having a di-tert-butylphenyl group as a bulky substituent instead of the substituent W (tri-tert-butylphenyl group). Therefore, it is believed that the aggregation inhibition effect due to the substituent W (tri-tert-butylphenyl group) provides improved PLOY, and Compound 1 may serve as a superior light-emitting dopant.

### Evaluation of OLED Devices

### Manufacture of Organic EL Devices

### Preparation of Materials for Forming Each Layer

As materials for forming each layer of organic EL devices, the following materials were prepared in addition to the obtained Compound 1, Compound 2, and Comparative Compound C1.

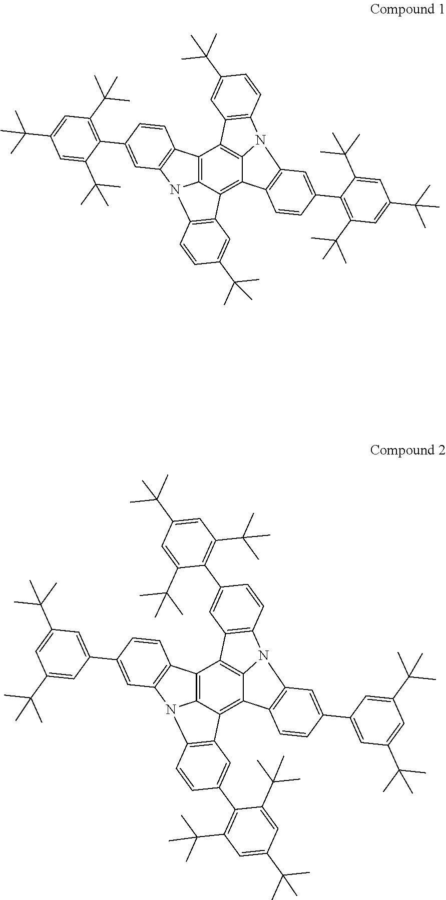

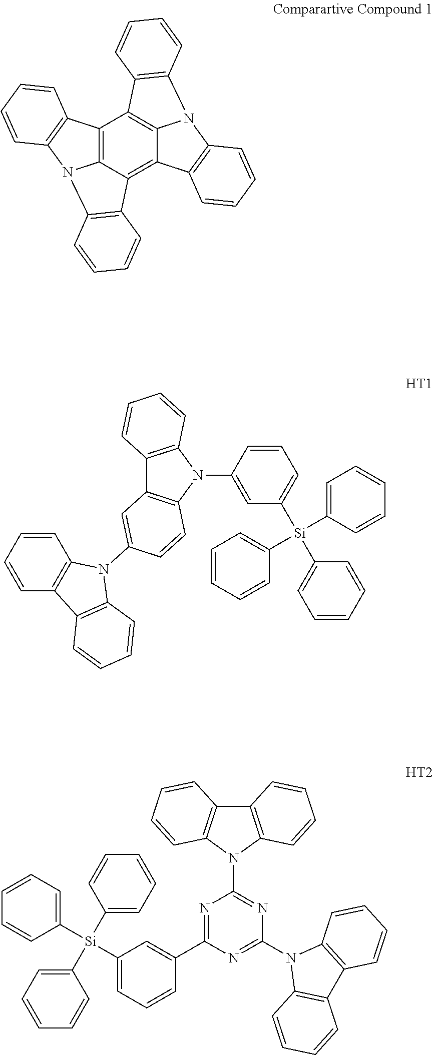 [SMILES: c1ccc2c(c1)c1c3c4ccccc4n4c5ccccc5c(c5c6ccccc6n2c15)c34 ; c1ccc([Si](c2ccccc2)(c2ccccc2)c2cccc(-n3c4ccccc4c4cc(-n5c6ccccc6c6ccccc65)ccc43)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

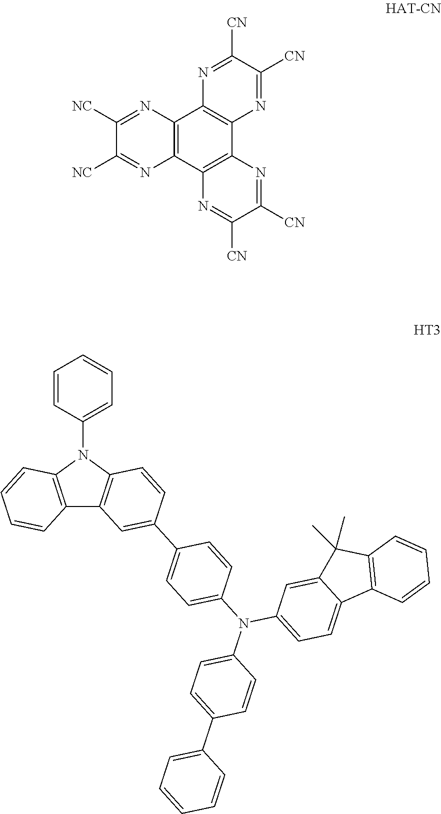 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21]

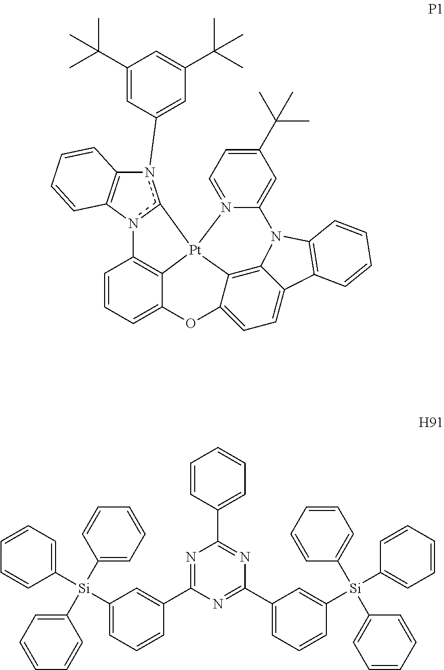

### Manufacture of Organic EL Device 1

### Example 1

An electrode-patterned ITO glass substrate was cut to a size of 50 mm×50 mm×0.7 mm, sonicated with acetone, isopropyl alcohol, and pure water, in this stated order, each for 15 minutes, and then cleaned by exposure to UV ozone for 30 minutes. The following layers were deposited on the ITO electrode (anode) of the glass substrate by using a vacuum deposition apparatus.

First, HAT-CN was deposited on the ITO electrode to form a hole injection layer having a thickness of 10 nm. Next, Compound HT3 was deposited on the hole injection layer to form a hole transport layer having a thickness of 140 nm. Then, Compound HT1 was deposited on the hole transport layer to form an electron blocking layer having a thickness of 5 nm. As a result, a hole transport region was formed.

Compound HT1, Compound HT2, and Compound 1 obtained above were co-deposited on the hole transport region obtained above to form an emission layer having a thickness of 40 nm. In this regard, the formation of the emission layer was performed such that the weight ratio of Compound HT1 and Compound HT2 in the emission layer was set to Compound HT1:Compound HT2=60:40. In addition, the formation of the emission layer was performed such that the concentration of Compound 1 was set to 1.5 wt % based on the total weight of Compound HT1, Compound HT2, and Compound 1 (that is, the total weight of the emission layer). In addition, Compound HT1 and Compound HT2 are host materials.

Compound HT2 was vacuum-deposited on the emission layer obtained above to form a hole blocking layer having a thickness of 5 nm. Next, Compound H91 and LiQ were co-deposited on the hole blocking layer at a weight ratio of Compound H91:LiQ=5:5 (unit: parts by weight) to form an electron transport layer having a thickness of 30 nm. Then, LiQ was deposited on the electron transport layer to form an electron injection layer having a thickness of 1 nm. As a result, an electron transport region was formed.

Aluminum (cathode) having a thickness of 100 nm was deposited on the electron injection layer obtained above to thereby manufacture an organic EL device.

Then, in a glove box under a nitrogen atmosphere with a water concentration of 1 ppm or less and an oxygen concentration of 1 ppm or less, a glass sealing tube with a desiccant and an ultraviolet curing resin (manufactured by MORESCO, product name WB90US) were used to seal the organic EL device manufactured in the above process. As a result, the manufacture of the organic EL device was completed.

### Example 2

An organic EL device was manufactured and sealed to prepare the organic EL device in the same manner as in Example 1, except that Compound 1 was replaced with Compound 2 in the emission layer during the formation of the emission layer of Example 1.

### Comparative Example 1

An organic EL device was manufactured and sealed to complete the organic EL device in the same manner as in Example 1, except that Compound 1 was replaced with Comparative Compound C1 in the emission layer during the formation of the emission layer of Example 1.

### Manufacture of Organic EL Device 2

### Example 3

An electrode-patterned ITO glass substrate was cut to a size of 50 mm×50 mm×0.7 mm, sonicated with acetone, isopropyl alcohol, and pure water, in this stated order, each for 15 minutes, and then cleaned by exposure to UV ozone for 30 minutes. The following layers were deposited on the ITO electrode (anode) of the glass substrate by using a vacuum deposition apparatus.

First, HAT-CN was deposited on the ITO electrode to form a hole injection layer having a thickness of 10 nm. Next, Compound HT3 was deposited on the hole injection layer to form a hole transport layer having a thickness of 140 nm. Then, Compound HT1 was deposited on the hole transport layer to form an electron blocking layer having a thickness of 5 nm. As a result, a hole transport region was formed.

Compound HT1, Compound HT2, Phosphorescent Complex P1, and Compound 1 obtained above were co-deposited on the hole transport region obtained above to form an emission layer having a thickness of 40 nm. In this regard, the formation of the emission layer was performed such that the weight ratio of Compound HT1, Compound HT2, and Phosphorescent Complex P1 in the emission layer was set to Compound HT1:Compound HT2:Phosphorescent Complex P1=60:40:13. In addition, the formation of the emission layer was performed such that the concentration of Compound 1 was set to 0.4 wt % based on the total weight of Compound HT1, Compound HT2, Phosphorescent Complex P1, and Compound 1 (that is, the total weight of the emission layer). In addition, Compound HT1 and Compound HT2 are host materials.

Compound HT2 was vacuum-deposited on the emission layer obtained above to form a hole blocking layer having a thickness of 5 nm. Next, Compound H91 and LiQ were co-deposited on the hole blocking layer at a weight ratio of Compound H91:LiQ=5:5 (unit: parts by weight) to form an electron transport layer having a thickness of 30 nm. Then, LiQ was deposited on the electron transport layer to form an electron injection layer having a thickness of 1 nm. As a result, an electron transport region was formed.

Aluminum (cathode) having a thickness of 100 nm was deposited on the electron injection layer to thereby manufacture an organic EL device.

Then, in a glove box under a nitrogen atmosphere with a water concentration of 1 ppm or less and an oxygen concentration of 1 ppm or less, a glass sealing tube with a desiccant and an ultraviolet curing resin (manufactured by MORESCO, product name WB90US) were used to seal the organic EL device manufactured in the above process. As a result, the manufacture of the organic EL device was completed.

### Example 4

An organic EL device was manufactured and sealed to complete the organic EL device in the same manner as in Example 3, except that Compound 1 was replaced with Compound 2 in the emission layer during the formation of the emission layer of Example 3.

### Comparative Example 2

An organic EL device was manufactured and sealed to complete the organic EL device in the same manner as in Example 3, except that Compound 1 was replaced with Comparative Compound C1 in the emission layer during the formation of the emission layer of Example 3.

### Evaluation of Organic EL Devices

For each of the organic EL devices of Examples 1 to 4 and Comparative Examples 1 and 2, the peak emission wavelength, emission spectrum width (FWHM) and external quantum yield at a luminance of 1,000 candela per square meter (cd/m²) were evaluated according to the following method.

### External Quantum Yield

Each organic EL device was allowed to emit light while a voltage applied thereto was changed using a DC constant voltage power supply (source meter 2400 manufactured by KEITHLEY), and the luminance, emission spectrum and luminescence amount at this time were measured using a luminance measurement device (multi-channel spectrometer PMA12 manufactured by Hamamatsu Photonics Co., Ltd.).

In this regard, the external quantum yield was calculated from an emission spectrum, a luminance, and a current value at the time of measurement. The external quantum yield at a luminance of 1,000 cd/m² was defined as EQE[%].

### Peak Emission Wavelength and Emission Spectrum Width (FWHM)

A peak emission wavelength and an emission spectrum width were determined from the measured emission spectrum.

In addition, in this evaluation, the peak emission wavelength is not particularly limited, but may be within a blue emission region, and may be about 455 nm to about 475 nm.

In this evaluation, it is considered that the smaller the emission spectrum width (FWHM), the better the color purity.

Results of evaluating the organic EL devices of Examples 1 to 4 and Comparative Examples 1 and 2 are shown in Tables 4 and 5.

TABLE 4
Evaluation of organic EL device (Manufacture 1)

| | | Peak | | |
|---|---|---|---|---|
| | | emission | | |
| | | wavelength | FWHM | |
| | Compound used in emission layer | [nm] | [nm] | EQE |
| Example 1 | 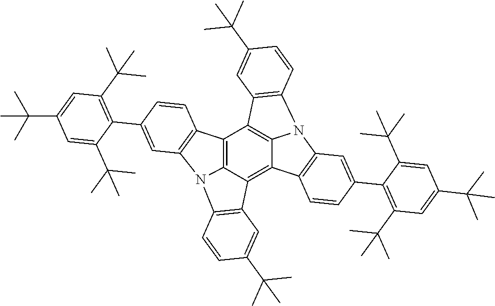 [SMILES: CC(C)(C)c1cc(C(C)(C)C)c(-c2ccc3c4c5c6cc(C(C)(C)C)ccc6n6c7cc(-c8c(C(C)(C)C)cc(C(C)(C)C)cc8C(C)(C)C)ccc7c(c7c8cc(C(C)(C)C)ccc8n(c3c2)c74)c56)c(C(C)(C)C)c1] | 459 | 15 | 1.4 |
| Example 2 | 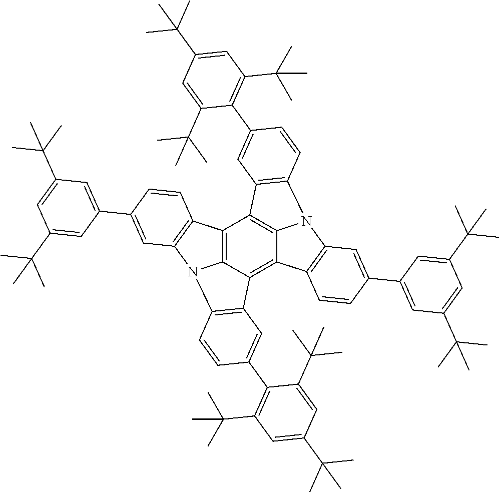 [SMILES: CC(C)(C)c1cc(-c2ccc3c4c5c6cc(-c7c(C(C)(C)C)cc(C(C)(C)C)cc7C(C)(C)C)ccc6n6c7cc(-c8cc(C(C)(C)C)cc(C(C)(C)C)c8)ccc7c(c7c8cc(-c9c(C(C)(C)C)cc(C(C)(C)C)cc9C(C)(C)C)ccc8n(c3c2)c74)c56)cc(C(C)(C)C)c1] | 466 | 18 | 1.7 |
| Comparative Example 1 | 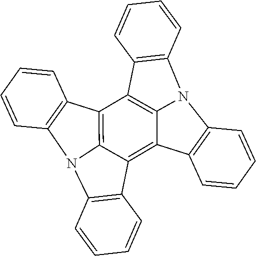 [SMILES: c1ccc2c(c1)c1c3c4ccccc4n4c5ccccc5c(c5c6ccccc6n2c15)c34] | 457 | 25 | 1.0 |

External quantum yield (EQE) is a relative value when the value of Comparative Example 1 is set to 1.

TABLE 5
Evaluation of organic EL device (Manufacture 2)

| | | Peak | | |
|---|---|---|---|---|
| | | emission | | |
| | | wavelength | FWHM | |
| | Compound used in emission layer | [nm] | [nm] | EQE |
| Example 3 | 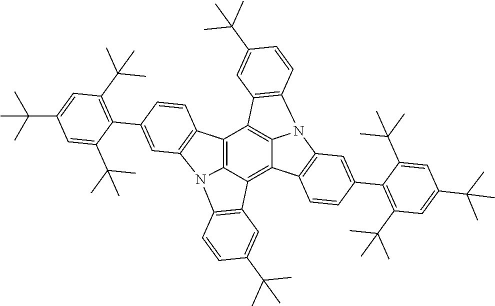 [SMILES: CC(C)(C)c1cc(C(C)(C)C)c(-c2ccc3c4c5c6cc(C(C)(C)C)ccc6n6c7cc(-c8c(C(C)(C)C)cc(C(C)(C)C)cc8C(C)(C)C)ccc7c(c7c8cc(C(C)(C)C)ccc8n(c3c2)c74)c56)c(C(C)(C)C)c1] | 461 | 18 | 2.0 |
| Example 4 | 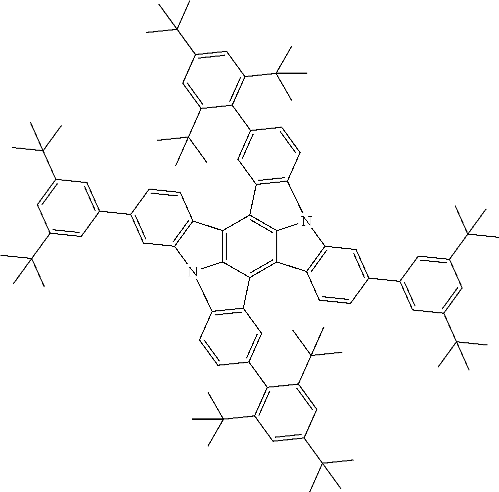 [SMILES: CC(C)(C)c1cc(-c2ccc3c4c5c6cc(-c7c(C(C)(C)C)cc(C(C)(C)C)cc7C(C)(C)C)ccc6n6c7cc(-c8cc(C(C)(C)C)cc(C(C)(C)C)c8)ccc7c(c7c8cc(-c9c(C(C)(C)C)cc(C(C)(C)C)cc9C(C)(C)C)ccc8n(c3c2)c74)c56)cc(C(C)(C)C)c1] | 465 | 18 | 2.3 |
| Comparative Example 2 | 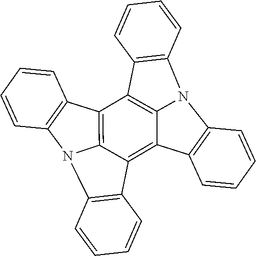 [SMILES: c1ccc2c(c1)c1c3c4ccccc4n4c5ccccc5c(c5c6ccccc6n2c15)c34] | 461 | 20 | 1.0 |

External quantum yield (EQE) is a relative value when the value of Comparative Example 2 is set to 1.

Each of the organic EL devices manufactured using Compounds 1 and 2 of the disclosure as luminescent materials had a greater external quantum yield than that of each of the organic EL devices manufactured using Comparative Compound C1 as a luminescent material. It is believed that a sterically bulky substituent in a compound of the disclosure contributes to efficiency improvement by inhibiting aggregation. In addition, the FWHM of each of Examples 1 to 4 was smaller than that of the corresponding Comparative Example 1 or 2, and thus the compound of the disclosure may serve as an excellent light-emitting dopant.

As described above, it was observed that in the organic EL devices of Examples 1 to 4, which included the compound according to the disclosure, the FWHM of the emission spectrum was narrower, luminescence with high color purity was realized, and the external quantum yield was also excellent. In other words, it was possible to manufacture blue electroluminescent devices with a narrower spectrum width, higher performance, higher efficiency, and higher color purity using the compound of the disclosure as a luminescent material, compared to devices using conventional luminescent materials.

As such, the compound according to the disclosure exhibited precisely tuned blue emission color, excellent color purity, and high luminescence efficiency in the organic EL devices. In addition, in particular, when the compound according to the disclosure is used in combination with a phosphorescent material, a significant improvement in luminescence efficiency was shown. It is considered that these results sufficiently satisfy the specifications required for future wide color gamut devices such as BT2100, making it possible to realize high-precision next-generation displays.

According to an aspect of the disclosure, it is possible to prepare a compound that has a peak wavelength in an emission spectrum within a blue wavelength region and is capable of providing luminescence with high color purity and high efficiency. In addition, according to another aspect of the disclosure, it is possible to provide an organic electroluminescent device including the compound. Furthermore, according to another aspect of the disclosure, it is possible to provide a means to obtain a peak wavelength in the emission spectrum within a blue wavelength region and with luminescence of a high color purity and high efficiency, in an organic electroluminescent device.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A compound represented by Formula (1): 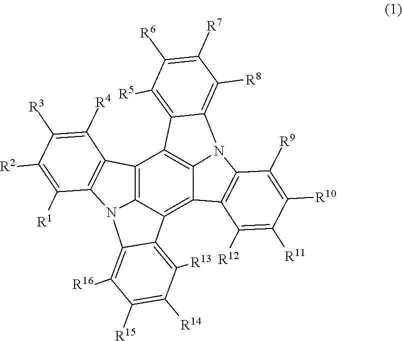
wherein, in Formula (1),
at least one of R¹ to R¹⁶ is a substituent W represented by Formula (2), wherein * indicates a binding site to a benzene ring: 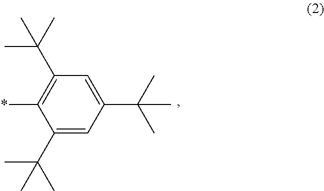 [SMILES: *c1c(C(C)(C)C)cc(C(C)(C)C)cc1C(C)(C)C]
and
R¹ to R¹⁶ other than the substituent W are each independently any one atom or group among (a1) to (a10):
(a1) a hydrogen or deuterium atom;
(a2) a halogen atom;
(a3) a cyano group;
(a4) a substituted or unsubstituted alkyl group having 1 to 20 carbon atoms;
(a5) a substituted or unsubstituted alkoxy group having 1 to 20 carbon atoms;
(a6) a substituted or unsubstituted arylamino group having 6 to 20 carbon atoms;
(a7) a substituted or unsubstituted triarylsilyl group, alkyldiarylsilyl group, dialkylarylsilyl group, or trialkylsilyl group, wherein an aryl group is an aryl group having 6 to 20 carbon atoms, and an alkyl group is an alkyl group having 1 to 20 carbon atoms;
(a8) a substituted or unsubstituted aromatic hydrocarbon group having 6 to 30 carbon atoms;
(a9) a substituted or unsubstituted heterocyclic group having 3 to 30 ring-forming atoms; and
(a10) a substituted or unsubstituted saturated hydrocarbon group or saturated heterocyclic group, each formed by bonding two adjacent groups among R¹ to R¹⁶ and having 5 to 9 ring-forming atoms.

2. The compound of claim 1, wherein at least two of R², R³, R⁶, R⁷, R¹⁰, R¹¹, R¹⁴, and R¹⁵ are each the substituent W.

3. The compound of claim 1, wherein a combination of R² and R¹⁰; R³ and R¹¹; R⁶ and R¹⁴; or R⁷ and R¹⁵ are each the substituent W.

4. The compound of claim 1, wherein R¹ to R¹⁶ other than the substituent W are each independently an atom or group of (a1), (a4), (a7), (a8), or (a10).

5. The compound of claim 1, wherein R¹ to R¹⁶ other than the substituent W comprise at least one group selected from Group X: 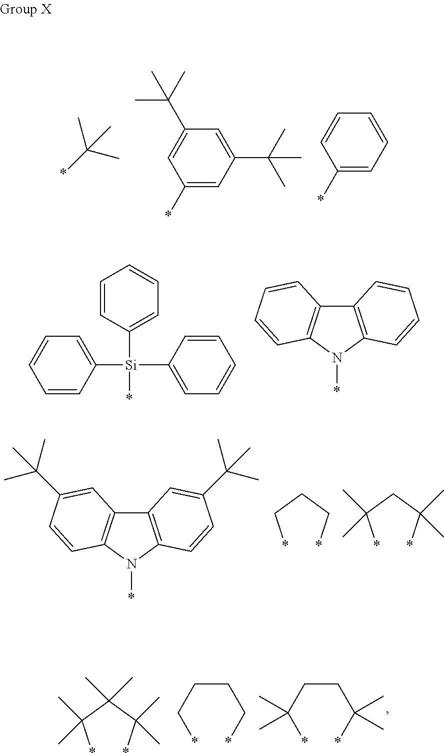
wherein, in the at least one group selected from the Group X,
* indicates a binding site to a benzene ring of Formula (1), and
a group having two * is bonded to two adjacent positions among R¹ to R¹⁶ and condensed with the benzene ring to form a condensed ring.

6. The compound of claim 5, wherein at least one of R¹ to R⁴ and at least one of R⁹ to R¹² are each the substituent W, and
at least one of R⁵ to R⁸ and at least one of R¹³ to R¹⁶ are each a group selected from the Group X.

7. The compound of claim 1, wherein R¹ to R¹⁶ other than the substituent W comprise at least one group selected from Group Y: 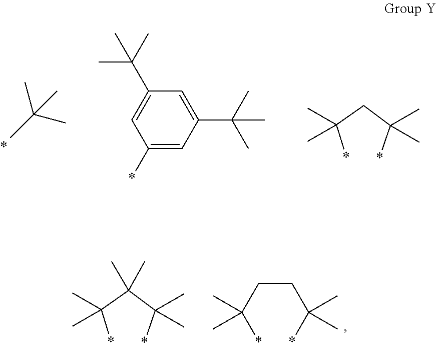
wherein, in the at least one group selected from the Group Y,
* indicates a binding site to a benzene ring of Formula (1), and
a group having two * is bonded to two adjacent positions among R¹ to R¹⁶ and condensed with a benzene ring to form a condensed ring.

8. The compound of claim 7, wherein at least one of R¹ to R⁴ and at least one of R⁹ to R¹² are each the substituent W, and
at least one of R⁵ to R⁸ and at least one of R¹³ to R¹⁶ are each a group selected from the Group Y.

9. The compound of claim 1, wherein the compound represented by Formula (1) is represented by any one of Compounds (100) to (117):

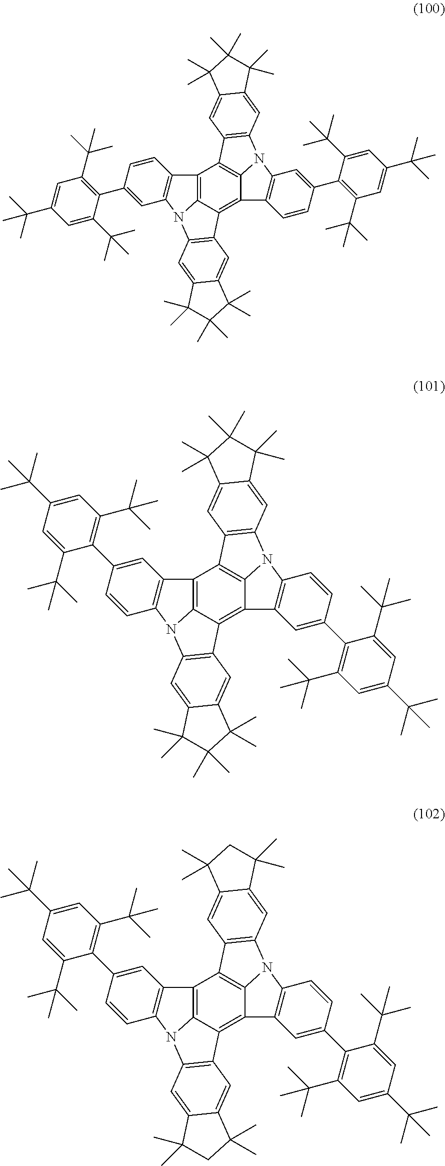

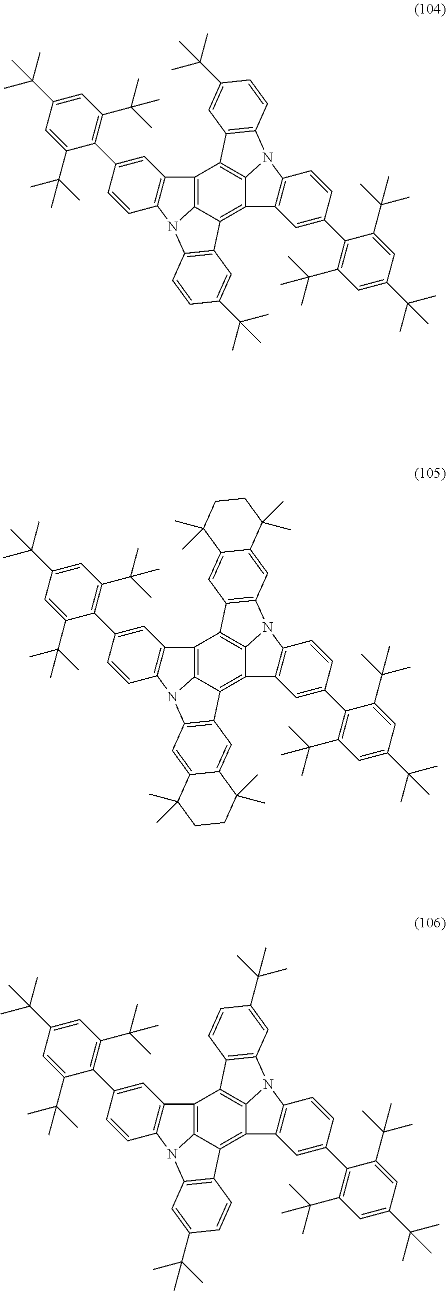

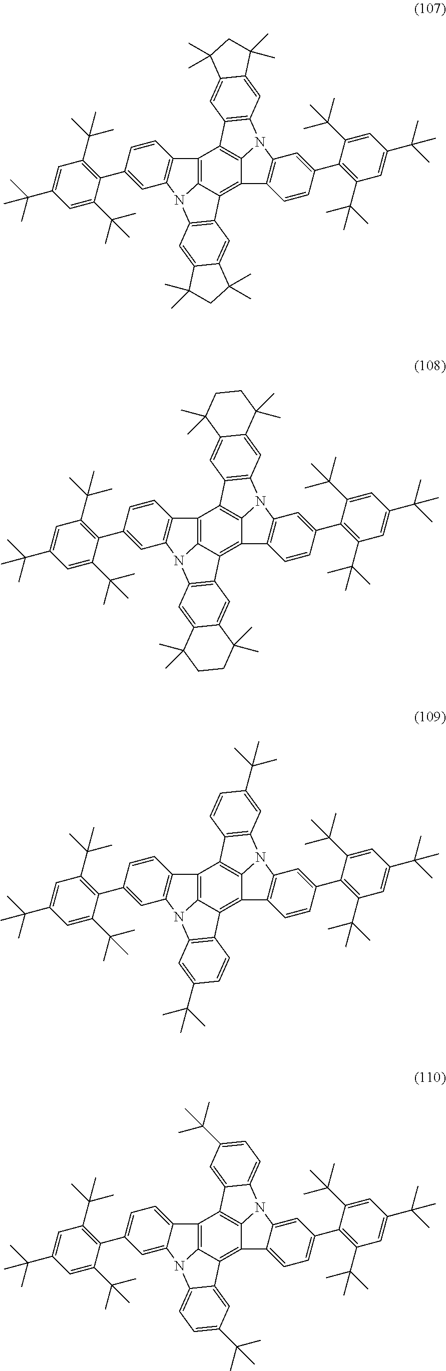

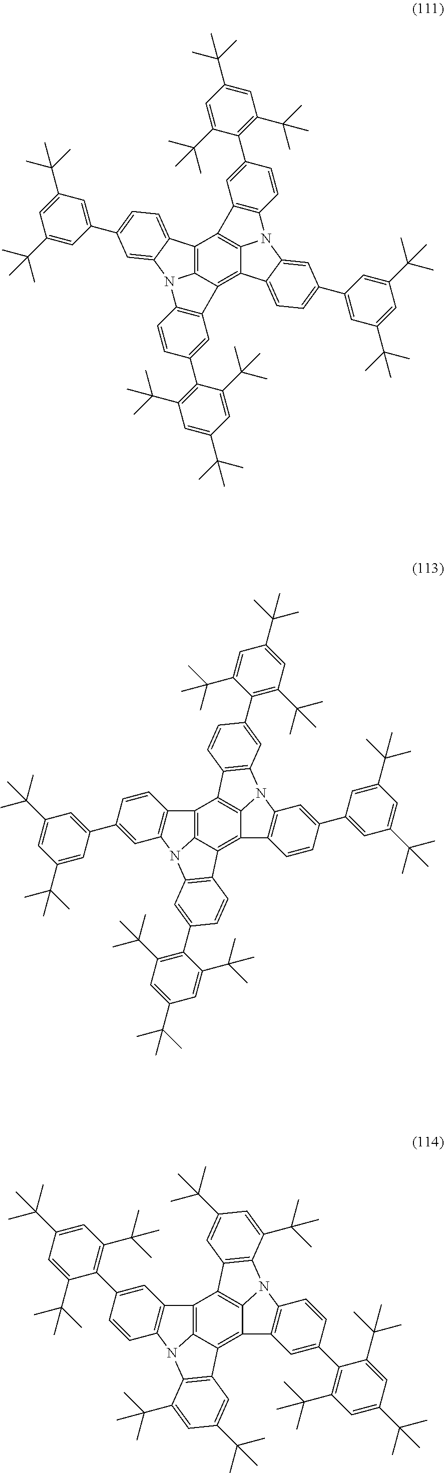

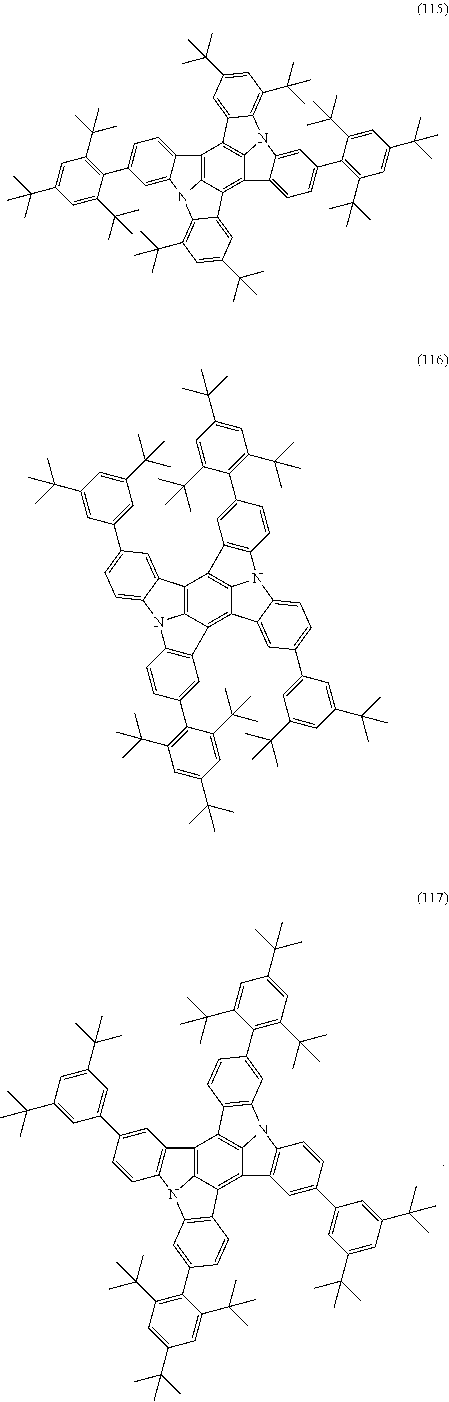

10. An organic electroluminescent device, comprising an emission layer comprising the compound of claim 1.

11. The organic electroluminescent device of claim 10, further comprising a phosphorescent complex in the emission layer.

12. The organic electroluminescent device of claim 11, wherein the phosphorescent complex is a platinum complex.

13. The organic electroluminescent device of claim 11, wherein the phosphorescent complex is a compound having a structure represented by Formula (4): 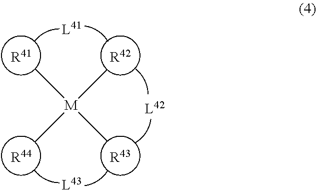
wherein M in Formula (4) is a metal ion having a coordination number of 4,
R⁴¹, R⁴², R⁴³, and R⁴⁴ are each independently a substituted or unsubstituted hydrocarbon cyclic group or a substituted or unsubstituted heterocyclic group,
L⁴¹ is a linking group linking R⁴¹ and R⁴²,
L⁴² is a linking group linking R⁴² and R⁴³, and
L⁴³ is a linking group linking R⁴³ and R⁴⁴.

14. The organic electroluminescent device of claim 10, wherein the emission layer further comprises a host material.

15. The organic electroluminescent device of claim 14, wherein the host material comprises a compound having a carbazole ring structure or a compound having a ring structure in which at least one ring-forming carbon atom of a carbazole ring is substituted with a nitrogen atom.

16. The organic electroluminescent device of claim 14, wherein the host material comprises a compound having a structure represented by Formula (5): 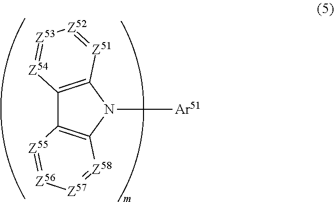
wherein, in Formula (5),
Z⁵¹ is CH, CR⁵¹, or N,
Z⁵² is CH, CR⁵², or N,
Z⁵⁸ is CH, CR⁵¹, or N,
Z⁵⁴ is CH, CR⁵⁴, or N,
Z⁵⁵ is CH, CR⁵⁵, or N,
Z⁵⁶ is CH, CR⁵⁶, or N,
Z⁵⁷ is CH, CR⁵⁷, or N,
Z⁵⁸ is CH, CR⁵⁸, or N,
R⁵¹ to R⁵⁸ are each independently a group of any one of (5a) to (5h):
(5a) a cyano group;
(5b) a substituted or unsubstituted alkyl group having 1 to 20 carbon atoms;
(5c) a substituted or unsubstituted alkoxy group having 1 to 20 carbon atoms;
(5d) a substituted or unsubstituted arylamino group having 6 to 20 carbon atoms;
(5e) a substituted or unsubstituted phosphoryl group;
(5f) a substituted or unsubstituted silyl group;
(5g) a substituted or unsubstituted monovalent aromatic hydrocarbon group; and
(5h) a substituted or unsubstituted monovalent heterocyclic group,
Ar⁵¹ is a group comprising at least one of an aromatic hydrocarbon group and a heterocyclic group, and
m is 1, 2, 3, 4, 5, or 6,
wherein R⁵¹ and R⁵², R⁵² and R⁵³, R⁵³ and R⁵⁴, R⁵⁵ and R⁵⁶, R⁵⁶ and R⁵⁷, R⁵⁷ and R⁵⁸, or a combination thereof optionally form an aliphatic hydrocarbon ring, an aromatic hydrocarbon ring, or a hetero ring, each including bonded carbon atoms.

17. The organic electroluminescent device of claim 14, wherein the host material comprises a compound having a triazine ring structure.

18. The organic electroluminescent device of claim 14, wherein the host material comprises a compound having a structure represented by Formula (6): 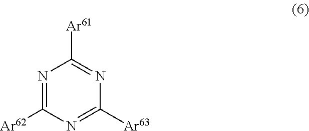
wherein, in Formula (6), Ar⁶¹ to Ar⁶³ are each independently a substituted or unsubstituted monovalent aromatic hydrocarbon group or a substituted or unsubstituted monovalent heterocyclic group.

19. The organic electroluminescent device of claim 10, wherein the emission layer further comprises a host material and a dopant material.

20. The organic electroluminescent device of claim 19, wherein the compound is the dopant material.
